SEQUENCE LISTING (91 sequences)

SEQ ID NO: 1 - DNA, 783 bp, Nodularia spumigena
  CDS (1)..(783)
atgttccagttagaacaaccaccattacctgaaattaaaatcactgctaccaccccagccgtgaaaagtaaatccctatttgggggcattttcatggcgatcgccattattagtatatgggctatcagcctaggtttgttactttatattgatatatcccaattcaagttttggatgttgttaccgctcatattttggcaaacatttttatatacgggattatttattacagctcatgatgccatgcatggggtagtttttcccaaaaatcccaaaatcaaccatttcattggctcattgtgcctgtttctttatggtcttttaccttatcaaaaacttttaaaaaagcattggctacatcaccataatccagccagtgaaacagatccagattttcacaacgggaagcagaaaaacttttttgcttggtatttatattttatgaagcgttactggagttggttacaaattatcacattaatgattatttataacttactaaaatatatatggcattttccagaggataatatgacttatttttgggtagttccctcaattttaagttctttacaattattttattttggaacttttctaccccacagtgagcctgtagaaggttataaagagcctcatcgttcccaaactattagccgtcccatttggtggtcatttataacttgttaccattttggttatcattacgaacatcatgaatacccccatgttccttggtggcaattaccagaaatttataaaatgtctaaatcaaatttgtga

SEQ ID NO: 2 - protein, 260 aa, Nodularia spumigena
MFQLEQPPLPEIKITATTPAVKSKSLFGGIFMAIAIISIWAISLGLLLYIDISQFKFWMLLPLIFWQTFLYTGLFITAHDAMHGVVFPKNPKINHFIGSLCLFLYGLLPYQKLLKKHWLHHHNPASETDPDFHNGKQKNFFAWYLYFMKRYWSWLQIITLMIIYNLLKYIWHFPEDNMTYFWVVPSILSSLQLFYFGTFLPHSEPVEGYKEPHRSQTISRPIWWSFITCYHFGYHYEHHEYPHVPWWQLPEIYKMSKSNL

SEQ ID NO: 3 - DNA, 783 bp, Nodularia spumigena
  CDS (1)..(783)
atgatccagttagaacaaccaccattacctgaaattaaaatcactgctaccaccccagccgtgaaaagtaaatccctatttgggggcattttcatggcgatcgccattattagtatatgggctatcagcctcggtttgttactttatattgatatatcccaattcaagttttggatgttgttgccaatcatcttttggcaaacatttttatatacgggattatttattacagctcatgatgccatgcacggggtagtttttcccaaaaatcctaaaatcaaccatttcattggctcattgtgcttgtttctttatggtcttttaccttatcaaaaacttttaaaaaagcattggctacatcaccataatccagccagtgaaacagatccagattttcacaacggtaagcagaaaaacttttttgcttggtatttatattttatgaagcgttactggagttggttacaaattatcacattaatgattatctataacgtggtaaaagctatatggcatcttcctgatgataatatgacttatttttgggtagttccctcaattttaagttctttacaattattttattttggaacttttttaccccatcgtgaacctgtagaaggttatcaagatcctcatcgttctcaaactattagccgtccaatttggtggtcattcataacttgttaccattttggttatcatcatgaacatcatgaataccctcatgttccttggtggcagttacctgaagtttatcaaatgtctaaatcaaatttgtga

SEQ ID NO: 4 - protein, 260 aa, Nodularia spumigena
MIQLEQPPLPEIKITATTPAVKSKSLFGGIFMAIAIISIWAISLGLLLYIDISQFKFWMLLPIIFWQTFLYTGLFITAHDAMHGVVFPKNPKINHFIGSLCLFLYGLLPYQKLLKKHWLHHHNPASETDPDFHNGKQKNFFAWYLYFMKRYWSWLQIITLMIIYNVVKAIWHLPDDNMTYFWVVPSILSSLQLFYFGTFLPHREPVEGYQDPHRSQTISRPIWWSFITCYHFGYHHEHHEYPHVPWWQLPEVYQMSKSNL

SEQ ID NO: 5 - DNA, 783 bp, Nodularia spumigena
  CDS (1)..(783)
atgatccagttagaacaaccaccattacctgaaattaaaatcactgctaccaccccagccgtgaaaagtaaatccctatttgggggcattttcatggcgatcgccattattagtatatgggctatcagcctgggtttgttactttatattgatatatcccaattcaacttttggatgttgttgccaatcatattttggcaaacatttttatatacgggattatttattacagctcatgatgccatgcacggggtagtttttcccaaaaatcctaaaatcaaccatttcattggctcattgtgcttgtttctttatggtcttttaccttatcaaaaacttttaaaaaagcattggctacatcaccataatccagccagtgaagcagatccagattttcacaacggtaagcagaaaaacttttttgcttggtatttatattttatgaagcgttactggagttggttacaaattatcacattaatgattatctttaacgtggtaaaatatatatggcatcttcctgatgataatctgacttatttttgggtagtgccatcaattttaagttccttacaattattttattttgggactttcttaccccatcgtgaacctgtagaaggttataaagatcctcatcgttctcaaactattagccgtccaatttggtggtcattcataacttgttaccattttggttatcatcatgaacatcacgaatacccccatgttccttggtggcaattacctgaaatttatcaaatgtctaaatcaaatttgtga

SEQ ID NO: 6 - protein, 260 aa, Nodularia spumigena
MIQLEQPPLPEIKITATTPAVKSKSLFGGIFMAIAIISIWAISLGLLLYIDISQFNFWMLLPIIFWQTFLYTGLFITAHDAMHGVVFPKNPKINHFIGSLCLFLYGLLPYQKLLKKHWLHHHNPASEADPDFHNGKQKNFFAWYLYFMKRYWSWLQIITLMIIFNVVKYIWHLPDDNLTYFWVVPSILSSLQLFYFGTFLPHREPVEGYKDPHRSQTISRPIWWSFITCYHFGYHHEHHEYPHVPWWQLPEIYQMSKSNL

SEQ ID NO: 7 - DNA, 783 bp, Nodularia spumigena; identical to SEQ ID NO: 5
  CDS (1)..(783)

SEQ ID NO: 8 - protein, 260 aa, Nodularia spumigena; identical to SEQ ID NO: 6

SEQ ID NO: 9 - DNA, 762 bp, Nostoc punctiforme
  CDS (1)..(762)
atgatccagttagaacaaccactgaaccatcaaacaaaactgaccccattagtaaaaaataaatctgcttttaggggcatttttattgctattgtcattattagtgtatggactattagcgagattttcttactttcgcttgatatctccaaattaaatatttggatgttatccgctttaatactttggcaaacatttttatatacgggattatttattacatctcatgatgctatgcatggggtagtgtttcccaaaaatcctaagattaatcgttttattggaacgttgagcttatctctttatggtcttttagcatatcaaaaactattgaaaaagcattggttacaccaccataatccagcgactgaaatagacccagatttccatgatggaaaacacaaaaatttcttcgcttggtattcttattttatgaagaactattggagttggggacaaatgattgccctaacttttatttatcactttgctagccacatccttcacataccgcatgaaaatctaatttcattttgggtttttccctcacttttaagttcattacagttattttattttggtacttatttaccccatagcgaaccaatagggggttatgttcaaccgcattgtgcagaaacgattagccgcccaatttggtggtcatttattacgtgctatcattttggctaccacaaagaacatcacgaatatcctcatgttccctggtggcagctcccagaaatttacaaagcaaaatag

SEQ ID NO: 10 - protein, 253 aa, Nostoc punctiforme
MIQLEQPLNHQTKLTPLVKNKSAFRGIFIAIVIISVWTISEIFLLSLDISKLNIWMLSALILWQTFLYTGLFITSHDAMHGVVFPKNPKINRFIGTLSLSLYGLLAYQKLLKKHWLHHHNPATEIDPDFHDGKHKNFFAWYSYFMKNYWSWGQMIALTFIYHFASHILHIPHENLISFWVFPSLLSSLQLFYFGTYLPHSEPIGGYVQPHCAETISRPIWWSFITCYHFGYHKEHHEYPHVPWWQLPEIYKAK

SEQ ID NO: 11 - DNA, 762 bp, Nostoc punctiforme
  CDS (1)..(762)
atgatccagttagaacaaccacccagttatcaaacaaaattgatttcaatagtgaaaagtaaatctcagtttaaaggacttttcattgctattgtcattgttagtgtatgggttattagcttgagtttattacttacccttgacatctcaaaattacaattttggatgttattgcctagcctagcttggcaaacatttttatacacgggattatttattacatctcatgatgctatgcatggggtagtatttccccaaaacagtaaaattaatcatcttattgggacattaaccctatctctttatggtcttttaccatataaaaaattattaaaaaagcattggttacaccaccaaaatccagcaactcaaatagacccagatttccataatggtaaacataaaaatttctttgcttggtatttccattttatgaagggttactggagttggggacaaattattgctctgacttttatctatcagtttgctaatcacatctttcatatacctcatgccaatctaataactttttgggtgcttccttcgcttttaagttcattccaattattttattttggtactttcctcccacatagtgaaccaatcgaaggctatattcagcctcattgtgcccaaactattagccgagcaattgggtggtcatttattacgtgctatcattttggctatcacgaagaacaccacgagtatcctcatattccttggtggcagttaccagaaatttacaaagcaaaatag

SEQ ID NO: 12 - protein, 253 aa, Nostoc punctiforme
MIQLEQPPSYQTKLISIVKSKSQFKGLFIAIVIVSVWVISLSLLLTLDISKLQFWMLLPSLAWQTFLYTGLFITSHDAMHGVVFPQNSKINHLIGTLTLSLYGLLPYKKLLKKHWLHHQNPATQIDPDFHNGKHKNFFAWYFHFMKGYWSWGQIIALTFIYQFANHIFHIPHANLITFWVLPSLLSSFQLFYFGTFLPHSEPIEGYIQPHCAQTISRAIGWSFITCYHFGYHEEHHEYPHIPWWQLPEIYKAK

SEQ ID NO: 13 - DNA, 771 bp, Gloeobacter violaceous
  CDS (1)..(771)
atgcgtggctcggcagtaaaggaacgtacttcgaagcggcttgccgaaggggttatcacccataagaacgattcttccggcctctggtgggctctggtgattatcggcctgtggatcttcagtttcgccgcagcgctgcgcttacctattggcgagttatcgctgcaggccgtcatcggcgtggtgatcctcagaacctttctgcacacaggtctatttatcactgcccacgacgcgatgcaccgaaccgtgtttcccgccaatcaccgcatcaacgattggcttggtaccgccgccgtcggtctgtacgcctttatgccctatcgcgaactactgattaaacatcagttgcaccaccgctttccagccaccggcaaagaccccgactaccacgacggcgaacatagcggcttctttcagtggtacttgaaattcatgaaggactatatggagagccggaacaccccgtttttgatcgcgggcatggccgtggtgttcggggtgtgcacttggctgatgggcgttccgctcgtcaacctggcgctgttctggttgttgccgctggtgctcagttccttgcaattgttctacttcggcacctacttgccccaccgacaacccgacggcggctaccgcaaccgtcaccgggccaccagcaaccgtctttcgagcttctggtcatttgtcagctgctatcacttcggctaccactgggagcaccacgaatacccgctcgttccctggcatcggctgcccgaggcgcgccgctag

SEQ ID NO: 14 - protein, 256 aa, Gloeobacter violaceous
MRGSAVKERTSKRLAEGVITHKNDSSGLWWALVIIGLWIFSFAAALRLPIGELSLQAVIGVVILRTFLHTGLFITAHDAMHRTVFPANHRINDWLGTAAVGLYAFMPYRELLIKHQLHHRFPATGKDPDYHDGEHSGFFQWYLKFMKDYMESRNTPFLIAGMAVVFGVCTWLMGVPLVNLALFWLLPLVLSSLQLFYFGTYLPHRQPDGGYRNRHRATSNRLSSFWSFVSCYHFGYHWEHHEYPLVPWHRLPEARR

SEQ ID NO: 15 - DNA, 1608 bp, Haematococcus pluvialis
  CDS (3)..(971)
ctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaaaaaaaaaaaaaaaaaaaaaaa

SEQ ID NO: 16 - protein, 322 aa, Haematococcus pluvialis
TFHKPVSGASALPHIGPPPHLHRSFAATTMLSKLQSISVKARRVELARDITRPKVCLHAQRCSLVRLRVAAPQTEEALGTVQAAGAGDEHSADVALQQLDRAIAERRARRKREQLSYQAAAIAASIGVSGIAIFATYLRFAMHMTVGGAVPWGEVAGTLLLVVGGALGMEMYARYAHKAIWHESPLGWLLHKSHHTPRTGPFEANDLFAIINGLPAMLLCTFGFWLPNVLGAACFGAGLGITLYGMAYMFVHDGLVHRRFPTGPIAGLPYMKRLTVAHQLHHSGKYGGAPWGMFLGPQELQHIPGAAEEVERLVLELDWSKR

SEQ ID NO: 17 - DNA, 1650 bp, Lycopersicon esculentum
  CDS (112)..(1614)
ggcacgaggaaacttttctctcttcactagctgtttacatgcttgaaatttcaagattttaggaccccatttgaagttttcttgaaacaaatattaccctgttggaaaaagatggatactttgttgaaaaccccaaataaccttgaatttctgaacccacatcatggttttgctgttaaagctagtacctttagatctgagaagcatcataattttggttctaggaagttttgtgaaactttgggtagaagtgtttgtgttaagggtagtagtagtgctcttttagagcttgtacctgagaccaaaaaggagaatcttgattttgagcttcctatgtatgacccttcaaaaggggttgttgtggatcttgctgtggttggtggtggccctgcaggacttgctgttgcacagcaagtttctgaagcaggactctctgtttgttcaattgatccgaatcctaaattgatatggcctaataactatggtgtttgggtggatgaatttgaggctatggacttgttagattgtctagatgctacctggtctggtgcagcagtgtacattgatgataatacggctaaagatcttcatagaccttatggaagggttaaccggaaacagctgaaatcgaaaatgatgcagaaatgtataatgaatggtgttaaattccaccaagccaaagttataaaggtgattcatgaggaatcgaaatccatgttgatatgcaatgatggtattactattcaggcaacggtggtgctcgatgcaactggcttctctagatctcttgttcagtatgataagccttataaccccgggtatcaagttgcttatggcattttggctgaagtggaagagcacccctttgatgtaaacaagatggttttcatggattggcgagattctcatttgaagaacaatactgatctcaaggagagaaatagtagaataccaacttttctttatgcaatgccattttcatccaacaggatatttcttgaagaaacatcactcgtagctcgtcctggcttgcgtatagatgatattcaagaacgaatggtggctcgtttaaaccatttggggataaaagtgaagagcattgaagaagatgaacattgtctaataccaatgggtggtccacttccagtattacctcagagagtcgttggaatcggtggtacagctggcatggttcatccatccaccggttatatggtggcaaggacactagctgcggctcctgttgttgccaatgccataattcaatacctcggttctgaaagaagtcattcgggtaatgaattatccacagctgtttggaaagatttgtggcctatagagaggagacgtcaaagagagttcttctgcttcggtatggatattcttctgaagcttgatttacctgctacaagaaggttctttgatgcattctttgacttagaacctcgttattggcatggcttcttatcgtctcgattgtttctacctgaactcatagtttttgggctgtctctattctctcatgcttcaaatacttctagatttgagataatgacaaagggaactgttccattagtaaatatgatcaacaatttgttacaggataaagaatgaatccgagtaattcggaatcttgtccaatctcgtgcc

SEQ ID NO: 18 - protein, 500 aa, Lycopersicon esculentum
MDTLLKTPNNLEFLNPHHGFAVKASTFRSEKHHNFGSRKFCETLGRSVCVKGSSSALLELVPETKKENLDFELPMYDPSKGVVVDLAVVGGGPAGLAVAQQVSEAGLSVCSIDPNPKLIWPNNYGVWVDEFEAMDLLDCLDATWSGAAVYIDDNTAKDLHRPYGRVNRKQLKSKMMQKCIMNGVKFHQAKVIKVIHEESKSMLICNDGITIQATVVLDATGFSRSLVQYDKPYNPGYQVAYGILAEVEEHPFDVNKMVFMDWRDSHLKNNTDLKERNSRIPTFLYAMPFSSNRIFLEETSLVARPGLRIDDIQERMVARLNHLGIKVKSIEEDEHCLIPMGGPLPVLPQRVVGIGGTAGMVHPSTGYMVARTLAAAPVVANAIIQYLGSERSHSGNELSTAVWKDLWPIERRRQREFFCFGMDILLKLDLPATRRFFDAFFDLEPRYWHGFLSSRLFLPELIVFGLSLFSHASNTSRFEIMTKGTVPLVNMINNLLQDKE

SEQ ID NO: 19 - DNA, 1779 bp, Arabidopsis thaliana
  CDS (1)..(1779)
atggatctccgtcggaggcctcctaaaccaccggttaccaacaacaacaactccaacggatctttccgttcttatcagcctcgcacttccgatgacgatcatcgtcgccgggctacaacaattgctcctccaccgaaagcatccgacgcgcttcctcttccgttatatctcacaaacgccgttttcttcacgctcttcttctccgtcgcgtattacctcctccaccggtggcgtgacaagatccgttacaatacgcctcttcacgtcgtcactatcacagaactcggcgccattattgctctcatcgcttcgtttatctatctcctagggttttttggtattgactttgttcagtcatttatctcacgtgcctctggtgatgcttgggatctcgccgatacgatcgatgatgatgaccaccgccttgtcacgtgctctccaccgactccgatcgtttccgttgctaaattacctaatccggaacctattgttaccgaatcgcttcctgaggaagacgaggagattgtgaaatcggttatcgacggagttattccatcgtactcgcttgaatctcgtctcggtgattgcaaaagagcggcgtcgattcgtcgtgaggcgttgcagagagtcaccgggagatcgattgaagggttaccgttggatggatttgattatgaatcgattttggggcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatga

SEQ ID NO: 20 - protein, 592 aa, Arabidopsis thaliana
MDLRRRPPKPPVTNNNNSNGSFRSYQPRTSDDDHRRRATTIAPPPKASDALPLPLYLTNAVFFTLFFSVAYYLLHRWRDKIRYNTPLHVVTITELGAIIALIASFIYLLGFFGIDFVQSFISRASGDAWDLADTIDDDDHRLVTCSPPTPIVSVAKLPNPEPIVTESLPEEDEEIVKSVIDGVIPSYSLESRLGDCKRAASIRREALQRVTGRSIEGLPLDGFDYESILGQCCEMPVGYIQIPVGIAGPLLLDGYEYSVPMATTEGCLVASTNRGCKAMFISGGATSTVLKDGMTRAPVVRFASARRASELKFFLENPENFDTLAVVFNRSSRFARLQSVKCTIAGKNAYVRFCCSTGDAMGMNMVSKGVQNVLEYLTDDFPDMDVIGISGNFCSDKKPAAVNWIEGRGKSVVCEAVIRGEIVNKVLKTSVAALVELNMLKNLAGSAVAGSLGGFNAHASNIVSAVFIATGQDPAQNVESSQCITMMEAINDGKDIHISVTMPSIEVGTVGGGTQLASQSACLNLLGVKGASTESPGMNARRLATIVAGAVLAGELSLMSAIAAGQLVRSHMKYNRSSRDISGATTTTTTTT

SEQ ID NO: 21 - DNA, 1401 bp, Arabidopsis thaliana
  CDS (1)..(1401): Arabidopsis thaliana ISPH
atggctgttgcgctccaattcagccgattatgcgttcgaccggatactttcgtgcgggagaatcatctctctggatccggatctctccgccgccggaaagctttatcagtccggtgctcgtctggcgatgagaacgctccttcgccatcggtggtgatggactccgatttcgacgccaaggtgttccgtaagaacttgacgagaagcgataattacaatcgtaaagggttcggtcataaggaggagacactcaagctcatgaatcgagagtacaccagtgatatattggagacactgaaaacaaatgggtatacttattcttggggagatgttactgtgaaactcgctaaagcatatggtttttgctggggtgttgagcgtgctgttcagattgcatatgaagcacgaaagcagtttccagaggagaggctttggattactaacgaaatcattcataacccgaccgtcaataagaggttggaagatatggatgttaaaattattccggttgaggattcaaagaaacagtttgatgtagtagagaaagatgatgtggttatccttcctgcgtttggagctggtgttgacgagatgtatgttcttaatgataaaaaggtgcaaattgttgacacgacttgtccttgggtgacaaaggtctggaacacggttgagaagcacaagaagggggaatacacatcagtaatccatggtaaatataatcatgaagagacgattgcaactgcgtcttttgcaggaaagtacatcattgtaaagaacatgaaagaggcaaattacgtttgtgattacattctcggtggccaatacgatggatctagctccacaaaagaggagttcatggagaaattcaaatacgcaatttcgaagggtttcgatcccgacaatgaccttgtcaaagttggtattgcaaaccaaacaacgatgctaaagggagaaacagaggagataggaagattactcgagacaacaatgatgcgcaagtatggagtggaaaatgtaagcggacatttcatcagcttcaacacaatatgcgacgctactcaagagcgacaagacgcaatctatgagctagtggaagagaagattgacctcatgctagtggttggcggatggaattcaagtaacacctctcaccttcaggaaatctcagaggcacggggaatcccatcttactggatcgatagtgagaaacggataggacctgggaataaaatagcctataagctccactatggagaactggtcgagaaggaaaactttctcccaaagggaccaataacaatcggtgtgacatcaggtgcatcaaccccggataaggtcgtggaagatgctttggtgaaggtgttcgacattaaacgtgaagagttattgcagctggcttga

SEQ ID NO: 22 - protein, 466 aa, Arabidopsis thaliana
MAVALQFSRLCVRPDTFVRENHLSGSGSLRRRKALSVRCSSGDENAPSPSVVMDSDFDAKVFRKNLTRSDNYNRKGFGHKEETLKLMNREYTSDILETLKTNGYTYSWGDVTVKLAKAYGFCWGVERAVQIAYEARKQFPEERLWITNEIIHNPTVNKRLEDMDVKIIPVEDSKKQFDVVEKDDVVILPAFGAGVDEMYVLNDKKVQIVDTTCPWVTKVWNTVEKHKKGEYTSVIHGKYNHEETIATASFAGKYIIVKNMKEANYVCDYILGGQYDGSSSTKEEFMEKFKYAISKGFDPDNDLVKVGIANQTTMLKGETEEIGRLLETTMMRKYGVENVSGHFISFNTICDATQERQDAIYELVEEKIDLMLVVGGWNSSNTSHLQEISEARGIPSYWIDSEKRIGPGNKIAYKLHYGELVEKENFLPKGPITIGVTSGASTPDKVVEDALVKVFDIKREELLQLA

SEQ ID NO: 23 - DNA, 2160 bp, Lycopersicon esculentum
  CDS (1)..(2160)
atggctttgtgtgcttatgcatttcctgggattttgaacaggactggtgtggtttcagattcttctaaggcaacccctttgttctctggatggattcatggaacagatctgcagtttttgttccaacacaagcttactcatgaggtcaagaaaaggtcacgtgtggttcaggcttccttatcagaatctggagaatactacacacagagaccgccaacgcctattttggacactgtgaactatcccattcatatgaaaaatctgtctctgaaggaacttaaacaactagcagatgaactaaggtcagatacaattttcaatgtatcaaagactgggggtcaccttggctcaagtcttggtgttgttgagctgactgttgctcttcattatgtcttcaatgcaccgcaagataggattctctgggatgttggtcatcagtcttatcctcacaaaatcttgactggtagaagggacaagatgtcgacattaaggcagacagatggtcttgcaggatttactaagcgatcggagagtgaatatgattgctttggcaccggccacagttccaccaccatctcagcaggcctagggatggctgttggtagagatctaaaaggaagaaacaacaatgttattgccgtaataggtgatggtgccatgacagcaggtcaagcttatgaagccatgaataatgctggttacctggactctgacatgattgttatcttaaacgacaatagacaagtttctttacctactgctactctggatgggccagttgctcctgttggagctctaagtagtgctttgagcaggttacagtctaataggcctctcagagaactaagagaagtcgcaaagggagttactaagcagattggtggtcctatgcatgagcttgctgcaaaagttgatgaatatgctcgtggcatgattagtggttctggatcaacattgtttgaagaacttggactttactatattggtcctgtggatggtcacaacattgatgatctaattgcgattctcaaagaggttagaagtactaaaacaacaggtccagtactgatccatgttgtcactgagaaaggcagaggttatccatatgctgagagagctgcagataagtatcatggagttgccaagtttgatccagcaacaggaaagcaattcaaagccagtgccaagacacagtcctatacaacatattttgccgaggctttaattgcagaagcagaagcagataaagacattgttgcaatccatgctgccatggggggtgggaccggaatgaaccttttccatcgtcgcttcccaacaaggtgttttgatgttggaatagcagaacaacatgcagtaacctttgctgctggattggcttgtgaaggcattaaacctttctgtgcaatctattcgtctttcatgcagagggcttatgaccaggtagtgcatgacgttgatttgcaaaagctgcccgtgaggtttgcaatggacagagcaggtcttgttggagcagatggtccaacacattgtggtgcatttgatgttacttacatggcatgtcttcctaacatggttgtaatggctccttctgatgaagcggagctatttcacatggtagcaactgctgccgccattgatgacagaccaagttgttttagatacccaagaggaaatgggatcggtgtagagcttccggctggaaacaaaggaattcctcttgaggttggtaaaggtaggatattgattgagggggagagagtggctctattgggatatggctcagcagtgcagaactgtttggatgctgctattgtgctagaatcccgcggcttacaagtaacagttgcagatgcacgtttctgcaaaccactggaccatgccctcataaggagccttgcaaaatcacatgaagtgctaatcactgtcgaagaaggatcaattggaggttttggatctcatgttgttcagttcatggccttagatgggcttcttgatggcaagttgaagtggagaccaatagttcttcctgatcgatacattgaccatggatctcctgttgatcagttggcggaagctggcctaacaccatctcacattgcagcaacagtatttaacatacttggacaaaccagagaggctctagaggtcatgacataa

SEQ ID NO: 24 - protein, 719 aa, Lycopersicon esculentum
MALCAYAFPGILNRTGVVSDSSKATPLFSGWIHGTDLQFLFQHKLTHEVKKRSRVVQASLSESGEYYTQRPPTPILDTVNYPIHMKNLSLKELKQLADELRSDTIFNVSKTGGHLGSSLGVVELTVALHYVFNAPQDRILWDVGHQSYPHKILTGRRDKMSTLRQTDGLAGFTKRSESEYDCFGTGHSSTTISAGLGMAVGRDLKGRNNNVIAVIGDGAMTAGQAYEAMNNAGYLDSDMIVILNDNRQVSLPTATLDGPVAPVGALSSALSRLQSNRPLRELREVAKGVTKQIGGPMHELAAKVDEYARGMISGSGSTLFEELGLYYIGPVDGHNIDDLIAILKEVRSTKTTGPVLIHVVTEKGRGYPYAERAADKYHGVAKFDPATGKQFKASAKTQSYTTYFAEALIAEAEADKDIVAIHAAMGGGTGMNLFHRRFPTRCFDVGIAEQHAVTFAAGLACEGIKPFCAIYSSFMQRAYDQVVHDVDLQKLPVRFAMDRAGLVGADGPTHCGAFDVTYMACLPNMVVMAPSDEAELFHMVATAAAIDDRPSCFRYPRGNGIGVELPAGNKGIPLEVGKGRILIEGERVALLGYGSAVQNCLDAAIVLESRGLQVTVADARFCKPLDHALIRSLAKSHEVLITVEEGSIGGFGSHVVQFMALDGLLDGKLKWRPIVLPDRYIDHGSPVDQLAEAGLTPSHIAATVFNILGQTREALEVMT

SEQ ID NO: 25 - DNA, 1434 bp, Arabidopsis thaliana
  CDS (1)..(1434)
atgatgacattaaactcactatctccagctgaatccaaagctatttctttcttggatacctccaggttcaatccaatccctaaactctcaggtgggtttagtttgaggaggaggaatcaagggagaggttttggaaaaggtgttaagtgttcagtgaaagtgcagcagcaacaacaacctcctccagcatggcctgggagagctgtccctgaggcgcctcgtcaatcttgggatggaccaaaacccatctctatcgttggatctactggttctattggcactcagacattggatattgtggctgagaatcctgacaaattcagagttgtggctctagctgctggttcgaatgttactctacttgctgatcaggtaaggagatttaagcctgcattggttgctgttagaaacgagtcactgattaatgagcttaaagaggctttagctgatttggactataaactcgagattattccaggagagcaaggagtgattgaggttgcccgacatcctgaagctgtaaccgttgttaccggaatagtaggttgtgcgggactaaagcctacggttgctgcaattgaagcaggaaaggacattgctcttgcaaacaaagagacattaatcgcaggtggtcctttcgtgcttccgcttgccaacaaacataatgtaaagattcttccggcagattcagaacattctgccatatttcagtgtattcaaggtttgcctgaaggcgctctgcgcaagataatcttgactgcatctggtggagcttttagggattggcctgtcgaaaagctaaaggaagttaaagtagcggatgcgttgaagcatccaaactggaacatgggaaagaaaatcactgtggactctgctacgcttttcaacaagggtcttgaggtcattgaagcgcattatttgtttggagctgagtatgacgatatagagattgtcattcatccgcaaagtatcatacattccatgattgaaacacaggattcatctgtgcttgctcaattgggttggcctgatatgcgtttaccgattctctacaccatgtcatggcccgatagagttccttgttctgaagtaacttggccaagacttgacctttgcaaactcggttcattgactttcaagaaaccagacaatgtgaaatacccatccatggatcttgcttatgctgctggacgagctggaggcacaatgactggagttctcagcgccgccaatgagaaagctgttgaaatgttcattgatgaaaagataagctatttggatatcttcaaggttgtggaattaacatgcgataaacatcgaaacgagttggtaacatcaccgtctcttgaagagattgttcactatgacttgtgggcacgtgaatatgccgcgaatgtgcagctttcttctggtgctaggccagttcatgcatga

SEQ ID NO: 26 - protein, 477 aa, Arabidopsis thaliana
MMTLNSLSPAESKAISFLDTSRFNPIPKLSGGFSLRRRNQGRGFGKGVKCSVKVQQQQQPPPAWPGRAVPEAPRQSWDGPKPISIVGSTGSIGTQTLDIVAENPDKFRVVALAAGSNVTLLADQVRRFKPALVAVRNESLINELKEALADLDYKLEIIPGEQGVIEVARHPEAVTVVTGIVGCAGLKPTVAAIEAGKDIALANKETLIAGGPFVLPLANKHNVKILPADSEHSAIFQCIQGLPEGALRKIILTASGGAFRDWPVEKLKEVKVADALKHPNWNMGKKITVDSATLFNKGLEVIEAHYLFGAEYDDIEIVIHPQSIIHSMIETQDSSVLAQLGWPDMRLPILYTMSWPDRVPCSEVTWPRLDLCKLGSLTFKKPDNVKYPSMDLAYAAGRAGGTMTGVLSAANEKAVEMFIDEKISYLDIFKVVELTCDKHRNELVTSPSLEEIVHYDLWAREYAANVQLSSGARPVHA

SEQ ID NO: 27 - DNA, 884 bp, Adonis palaestina
  CDS (180)..(884): Adonis palaestina clone ApIPI28
cgtcgatcaggattaatcctttatatagtatcttctccaccaccactaaaacattatcagcttcgtgttcttctcccgctgttcatcttcagcagcgttgtcgtactctttctatttcttcttccatcactaacagtcctcgccgagggttgaatcggctgttcgcctcaacgtcgactatgggtgaagtcgctgatgctggtatggatgccgtccagaagcggcttatgttcgacgatgaatgtattttggtggatgagaatgacaaggtcgtcggacatgattccaaatacaactgtcatttgatggaaaagatagaggcagaaaacttgcttcacagagccttcagtgttttcttattcaactcaaaatacgagttgcttcttcagcaacgatctgcaacgaaggtaacattcccgctcgtatggacaaacacctgttgcagccatcccctcttccgtgattccgaactcatagaagaaaattttctcggggtacgaaacgctgcacaaaggaagcttttagacgagctaggcattccagctgaagacgtaccagttgatgaattcactcctcttggtcgcattctttacaaagctccatctgacggaaaatggggagagcacgaactggactatcttctgtttattgtccgagatgtgaaatacgatccaaacccagatgaagttgctgacgctaagtacgttaatcgcgaggagttgaaagagatactgagaaaagctgatgcaggtgaagagggaataaagttgtctccttggtttagattggttgtggataactttttgttcaagtggtgggatcatgtagaggaggggaagattaaggacgtcgccgacatgaaaactatccacaagttgacttaa

SEQ ID NO: 28 - protein, 234 aa, Adonis palaestina
MGEVADAGMDAVQKRLMFDDECILVDENDKVVGHDSKYNCHLMEKIEAENLLHRAFSVFLFNSKYELLLQQRSATKVTFPLVWTNTCCSHPLFRDSELIEENFLGVRNAAQRKLLDELGIPAEDVPVDEFTPLGRILYKAPSDGKWGEHELDYLLFIVRDVKYDPNPDEVADAKYVNREELKEILRKADAGEEGIKLSPWFRLVVDNFLFKWWDHVEEGKIKDVADMKTIHKLT

SEQ ID NO: 29 - DNA, 1402 bp, Arabidopsis thaliana
  CDS (52)..(1317)
aagtctttgcctctttggtttactttcctctgttttcgatccatttagaaaatgttattcacgaggagtgttgctcggatttcttctaagtttctgagaaaccgtagcttctatggctcctctcaatctctcgcctctcatcggttcgcaatcattcccgatcagggtcactcttgttctgactctccacacaagggttacgtttgcagaacaacttattcattgaaatctccggtttttggtggatttagtcatcaactctatcaccagagtagctccttggttgaggaggagcttgacccattttcgcttgttgccgatgagctgtcacttcttagtaataagttgagagagatggtacttgccgaggttccaaagcttgcctctgctgctgagtacttcttcaaaaggggtgtgcaaggaaaacagtttcgttcaactattttgctgctgatggcgacagctctggatgtacgagttccagaagcattgattggggaatcaacagatatagtcacatcagaattacgcgtaaggcaacggggtattgctgaaatcactgaaatgatacacgtcgcaagtctactgcacgatgatgtcttggatgatgccgatacaaggcgtggtgttggttccttaaatgttgtaatgggtaacaagatgtcggtattagcaggagacttcttgctctcccgggcttgtggggctctcgctgctttaaagaacacagaggttgtagcattacttgcaactgctgtagaacatcttgttaccggtgaaaccatggagataactagttcaaccgagcagcgttatagtatggactactacatgcagaagacatattataagacagcatcgctaatctctaacagctgcaaagctgttgccgttctcactggacaaacagcagaagttgccgtgttagcttttgagtatgggaggaatctgggtttagcattccaattaatagacgacattcttgatttcacgggcacatctgcctctctcggaaagggatcgttgtcagatattcgccatggagtcataacagccccaatcctctttgccatggaagagtttcctcaactacgcgaagttgttgatcaagttgaaaaagatcctaggaatgttgacattgctttagagtatcttgggaagagcaagggaatacagagggcaagagaattagccatggaacatgcgaatctagcagcagctgcaatcgggtctctacctgaaacagacaatgaagatgtcaaaagatcgaggcgggcacttattgacttgacccatagagtcatcaccagaaacaagtgagattaagtaatgtttctctctatacaccaaaacattcctcatttcatttgtaggattttgttggtccaattcgtttcacgaa

SEQ ID NO: 30 - protein, 422 aa, Arabidopsis thaliana
MLFTRSVARISSKFLRNRSFYGSSQSLASHRFAIIPDQGHSCSDSPHKGYVCRTTYSLKSPVFGGFSHQLYHQSSSLVEEELDPFSLVADELSLLSNKLREMVLAEVPKLASAAEYFFKRGVQGKQFRSTILLLMATALDVRVPEALIGESTDIVTSELRVRQRGIAEITEMIHVASLLHDDVLDDADTRRGVGSLNVVMGNKMSVLAGDFLLSRACGALAALKNTEVVALLATAVEHLVTGETMEITSSTEQRYSMDYYMQKTYYKTASLISNSCKAVAVLTGQTAEVAVLAFEYGRNLGLAFQLIDDILDFTGTSASLGKGSLSDIRHGVITAPILFAMEEFPQLREVVDQVEKDPRNVDIALEYLGKSKGIQRARELAMEHANLAAAAIGSLPETDNEDVKRSRRALIDLTHRVITRNK

SEQ ID NO: 31 - DNA, 1155 bp, Arabidopsis thaliana
  CDS (1)..(1155)
atgagtgtgagttgttgttgtaggaatctgggcaagacaataaaaaaggcaataccttcacatcatttgcatctgagaagtcttggtgggagtctctatcgtcgtcgtatccaaagctcttcaatggagaccgatctcaagtcaacctttctcaacgtttattctgttctcaagtctgaccttcttcatgacccttccttcgaattcaccaatgaatctcgtctctgggttgatcggatgctggactacaatgtacgtggagggaaactcaatcggggtctctctgttgttgacagtttcaaacttttgaagcaaggcaatgatttgactgagcaagaggttttcctctcttgtgctctcggttggtgcattgaatggctccaagcttatttccttgtgcttgatgatattatggataactctgtcactcgccgtggtcaaccttgctggttcagagttcctcaggttggtatggttgccatcaatgatgggattctacttcgcaatcacatccacaggattctcaaaaagcatttccgtgataagccttactatgttgaccttgttgatttgtttaatgaggttgagttgcaaacagcttgtggccagatgatagatttgatcaccacctttgaaggagaaaaggatttggccaagtactcattgtcaatccaccgtcgtattgtccagtacaaaacggcttattactcattttatctccctgttgcttgtgcgttgcttatggcgggcgaaaatttggaaaaccatattgacgtgaaaaatgttcttgttgacatgggaatctacttccaagtgcaggatgattatctggattgttttgctgatcccgagacgcttggcaagataggaacagatatagaagatttcaaatgctcgtggttggtggttaaggcattagagcgctgcagcgaagaacaaactaagatattatatgagaactatggtaaacccgacccatcgaacgttgctaaagtgaaggatctctacaaagagctggatcttgagggagttttcatggagtatgagagcaaaagctacgagaagctgactggagcgattgagggacaccaaagtaaagcaatccaagcagtgctaaaatccttcttggctaagatctacaagaggcagaagtag

SEQ ID NO: 32 - protein, 384 aa, Arabidopsis thaliana
MSVSCCCRNLGKTIKKAIPSHHLHLRSLGGSLYRRRIQSSSMETDLKSTFLNVYSVLKSDLLHDPSFEFTNESRLWVDRMLDYNVRGGKLNRGLSVVDSFKLLKQGNDLTEQEVFLSCALGWCIEWLQAYFLVLDDIMDNSVTRRGQPCWFRVPQVGMVAINDGILLRNHIHRILKKHFRDKPYYVDLVDLFNEVELQTACGQMIDLITTFEGEKDLAKYSLSIHRRIVQYKTAYYSFYLPVACALLMAGENLENHIDVKNVLVDMGIYFQVQDDYLDCFADPETLGKIGTDIEDFKCSWLVVKALERCSEEQTKILYENYGKPDPSNVAKVKDLYKELDLEGVFMEYESKSYEKLTGAIEGHQSKAIQAVLKSFLAKIYKRQK

SEQ ID NO: 33 - DNA, 1101 bp, Sinabs alba
  CDS (1)..(1101)
atggcttcttcagtgactcctctaggttcatgggttcttcttcaccatcatccttcaactatcttaacccaatccagatccagatctcctccttctctcatcacccttaaacccatctccctcactccaaaacgcaccgtttcgtcttcttcctcctcttccctcatcaccaaagaagacaacaacctcaaatcctcttcctcttccttcgatttcatgtcttacatcatccgcaaagccgactccgtcaacaaagccttagactccgccgtccctctccgggagccactcaagatccacgaagcgatgcgttactctctcctcgccggaggaaaacgcgtcagaccagttctctgcatcgccgcgtgcgagctagtcggaggagaagagtctttagctatgccggcgcgttgcgccgtggaaatgatccacaccatgtcgttgatccacgacgacttgccttgtatggataacgacgatctccgccgcggaaagcccacgaatcacaaagtttacggcgaagacgtggcggttttagccggagacgcgcttctttcgttcgccttcgagcatttagcgtcggctacgagctcggaggtttctccggcgagagtggttagagctgtgggagagttggctaaagccatcggcaccgaagggctcgtggcgggacaagtggtggatataagcagtgaagggttggacttaaacaacgtcggattggagcatttgaagtttatacatttgcataaaacggcggcgttgcttgaagcttcagcggttttgggtgggatcatcggtggagggagtgatgaagagatcgagaggctgaggaagttcgcgaggtgtattgggttgttgtttcaggtggttgatgatatcttggacgtgacgaaatcgtctcaagaactggggaaaaccgctgggaaagatttgattgctgataagttgacttatccgaagctcatgggtttggagaaatcgagagagttcgctgagaagttgaatacagaggcacgtgatcagcttttagggtttgattccgacaaggttgctcctttgttggctttggctaattacattgccaatagacagaactga

SEQ ID NO: 34 - protein, 366 aa, Sinabs alba
MASSVTPLGSWVLLHHHPSTILTQSRSRSPPSLITLKPISLTPKRTVSSSSSSSLITKEDNNLKSSSSSFDFMSYIIRKADSVNKALDSAVPLREPLKIHEAMRYSLLAGGKRVRPVLCIAACELVGGEESLAMPARCAVEMIHTMSLIHDDLPCMDNDDLRRGKPTNHKVYGEDVAVLAGDALLSFAFEHLASATSSEVSPARVVRAVGELAKAIGTEGLVAGQVVDISSEGLDLNNVGLEHLKFIHLHKTAALLEASAVLGGIIGGGSDEEIERLRKFARCIGLLFQVVDDILDVTKSSQELGKTAGKDLIADKLTYPKLMGLEKSREFAEKLNTEARDQLLGFDSDKVAPLLALANYIANRQN

SEQ ID NO: 35 - DNA, 930 bp, Erwinia uredovora
  CDS (1)..(930)
atgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctag

SEQ ID NO: 36 - protein, 309 aa, Erwinia uredovora
MNNPSLLNHAVETMAVGSKSFATASKLFDAKTRRSVLMLYAWCRHCDDVIDDQTLGFQARQPALQTPEQRLMQLEMKTRQAYAGSQMHEPAFAAFQEVAMAHDIAPAYAFDHLEGFAMDVREAQYSQLDDTLRYCYHVAGVVGLMMAQIMGVRDNATLDRACDLGLAFQLTNIARDIVDDAHAGRCYLPASWLEHEGLNKENYAAPENRQALSRIARRLVQEAEPYYLSATAGLAGLPLRSAWAIATAKQVYRKIGVKVEQAGQQAWDQRQSTTTPEKLTLLLAASGQALTSRMRAHPPRPAHLWQRPL

SEQ ID NO: 37 - DNA, 1479 bp, Erwinia uredovora
  CDS (1)..(1479)
atgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatga

SEQ ID NO: 38 - protein, 492 aa, Erwinia uredovora
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFAYLEQHYMPGLRSQLVTHRMFTPFDFRDQLNAYHGSAFSVEPVLTQSAWFRPHNRDKTITNLYLVGAGTHPGAGIPGVIGSAKATAGLMLEDLI

SEQ ID NO: 39 - DNA, 1725 bp, Narcissus pseudonarcissus
  CDS (1)..(1725)
atggcttcttccacttgtttaattcattcttcctcttttggggttggaggaaagaaagtgaagatgaacacgatgattcgatcgaagttgttttcaattcggtcggctttggacactaaggtgtctgatatgagcgtcaatgctccaaaaggattgtttccaccagagcctgagcactacagggggccaaagcttaaagtggctatcattggagctgggctcgctggcatgtcaactgcagtggagcttttggatcaagggcatgaggttgacatatatgaatccagacaatttattggtggtaaagtcggttcttttgtagataagcgtggaaaccatattgaaatgggactccatgtgttttttggttgctataacaatcttttcagacttatgaaaaaggtaggtgcagatgaaaatttactggtgaaggatcatactcatacctttgtaaaccgaggtggagaaattggtgaacttgatttccgacttccgatgggtgcaccattacatggtattcgtgcatttctaacaactaatcaactgaagccttatgataaagcaaggaatgctgtggctcttgcccttagcccagttgtacgtgctcttattgatccaaatggtgcaatgcaggatataaggaacttagataatattagcttttctgattggttcttatccaaaggcggtacccgcatgagcatccaaaggatgtgggatccagttgcttatgccctcggatttattgactgtgataatatcagtgcccgttgtatgcttactatattttctctatttgctactaagacagaagcttctctgttgcgtatgttgaagggttcgcctgatgtttacttaagcggtcctataagaaagtatattacagataaaggtggaaggtttcacctaaggtgggggtgtagagagatactttatgatgaactatcaaatggcgacacatatatcacaggcattgcaatgtcgaaggctaccaataaaaaacttgtgaaagctgacgtgtatgttgcagcatgtgatgttcctggaataaaaaggttgatcccatcggagtggagagaatgggatctatttgacaatatctataaactagttggagttccagttgtcactgttcagcttaggtacaatggttgggtgacagagatgcaagatctggaaaaatcaaggcagttgagagctgcagtaggattggataatcttctttatactccagatgcagacttttcttgtttttctgatcttgcactctcgtcgcctgaagattattatattgaaggacaagggtccctaatacaggctgttctcacgccaggggatccatacatgcccctacctaatgatgcaattatagaaagagttcggaaacaggttttggatttattcccatcctctcaaggcctggaagttctatggtcttcggtggttaaaatcggacaatccctatatcgggaggggcctggaaaggacccattcagacctgatcagaagacaccagtaaaaaatttcttccttgcaggttcatacaccaaacaggattacattgacagtatggaaggagcgaccctatcggggagacaagcagctgcatatatctgcagcgccggtgaagatctggcagcacttcgcaagaagatcgctgctgatcatccagagcaactgatcaacaaagattctaacgtgtcggatgaactgagtctcgtataa

SEQ ID NO: 40 - protein, 574 aa, Narcissus pseudonarcissus
MASSTCLIHSSSFGVGGKKVKMNTMIRSKLFSIRSALDTKVSDMSVNAPKGLFPPEPEHYRGPKLKVAIIGAGLAGMSTAVELLDQGHEVDIYESRQFIGGKVGSFVDKRGNHIEMGLHVFFGCYNNLFRLMKKVGADENLLVKDHTHTFVNRGGEIGELDFRLPMGAPLHGIRAFLTTNQLKPYDKARNAVALALSPVVRALIDPNGAMQDIRNLDNISFSDWFLSKGGTRMSIQRMWDPVAYALGFIDCDNISARCMLTIFSLFATKTEASLLRMLKGSPDVYLSGPIRKYITDKGGRFHLRWGCREILYDELSNGDTYITGIAMSKATNKKLVKADVYVAACDVPGIKRLIPSEWREWDLFDNIYKLVGVPVVTVQLRYNGWVTEMQDLEKSRQLRAAVGLDNLLYTPDADFSCFSDLALSSPEDYYIEGQGSLIQAVLTPGDPYMPLPNDAIIERVRKQVLDLFPSSQGLEVLWSSVVKIGQSLYREGPGKDPFRPDQKTPVKNFFLAGSYTKQDYIDSMEGATLSGRQAAAYICSAGEDLAALRKKIAADHPEQLINKDSNVSDELSLV

SEQ ID NO: 41 - DNA, 1848 bp, Lycopersicon esculentum
  CDS (1)..(1848)
atgtgtaccttgagttttatgtatcctaattcacttcttgatggtacctgcaagactgtagctttgggtgatagcaaaccaagatacaataaacagagaagttcttgttttgaccctttgataattggaaattgtactgatcagcagcagctttgtggcttgagttggggggtggacaaggctaagggaagaagagggggtactgtttccaatttgaaagcagttgtagatgtagacaaaagagtggagagctatggcagtagtgatgtagaaggaaatgagagtggcagctatgatgccattgttataggttcaggaataggtggattggtggcagcgacgcagctggcggttaagggagctaaggttttagttctggagaagtatgttattcctggtggaagctctggcttttacgagagggatggttataagtttgatgttggttcatcagtgatgtttggattcagtgataagggaaacctcaatttaattactcaagcattggcagcagtaggacgtaaattagaagttatacctgacccaacaactgtacatttccacctgccaaatgacctttctgttcgtatacaccgagagtatgatgacttcattgaagagcttgtgagtaaatttccacatgaaaaggaagggattatcaaattttacagtgaatgctggaagatctttaattctctgaattcattggaactgaagtctttggaggaacccatctacctttttggccagttctttaagaagccccttgaatgcttgactcttgcctactatttgccccagaatgctggtagcatcgctcggaagtatataagagatcctgggttgctgtcttttatagatgcagagtgctttatcgtgagtacagttaatgcattacaaacaccaatgatcaatgcaagcatggttctatgtgacagacattttggcggaatcaactaccccgttggtggagttggcgagatcgccaaatccttagcaaaaggcttggatgatcacggaagtcagatactttatagggcaaatgttacaagtatcattttggacaatggcaaagctgtgggagtgaagctttctgacgggaggaagttttatgctaaaaccatagtatcgaatgctaccagatgggatacttttggaaagcttttaaaagctgagaatctgccaaaagaagaagaaaatttccagaaagcttatgtaaaagcaccttcttttctttctattcatatgggagttaaagcagatgtactcccaccagacacagattgtcaccattttgtcctcgaggatgattggacaaatttggagaaaccatatggaagtatattcttgagtattccaacagttcttgattcctcattggccccagaaggacaccatattcttcacatttttacaacatcgagcattgaagattgggagggactctctccgaaagactatgaagcgaagaaagaggttgttgctgaaaggattataagcagacttgaaaaaacactcttcccagggcttaagtcatctattctctttaaggaggtgggaactccaaagacccacagacgataccttgctcgtgatagtggtacctatggaccaatgccacgcggaacacctaagggactcctgggaatgcctttcaataccactgctatagatggtctatattgtgttggcgatagttgcttcccaggacaaggtgttatagctgtagccttttcaggagtaatgtgcgctcatcgtgttgcagctgacttagggtttgaaaaaaaatcagatgtgctggacagtgctcttcttagactacttggttggttaaggacactagcatga

SEQ ID NO: 42 - protein, 615 aa, Lycopersicon esculentum
MCTLSFMYPNSLLDGTCKTVALGDSKPRYNKQRSSCFDPLIIGNCTDQQQLCGLSWGVDKAKGRRGGTVSNLKAVVDVDKRVESYGSSDVEGNESGSYDAIVIGSGIGGLVAATQLAVKGAKVLVLEKYVIPGGSSGFYERDGYKFDVGSSVMFGFSDKGNLNLITQALAAVGRKLEVIPDPTTVHFHLPNDLSVRIHREYDDFIEELVSKFPHEKEGIIKFYSECWKIFNSLNSLELKSLEEPIYLFGQFFKKPLECLTLAYYLPQNAGSIARKYIRDPGLLSFIDAECFIVSTVNALQTPMINASMVLCDRHFGGINYPVGGVGEIAKSLAKGLDDHGSQILYRANVTSIILDNGKAVGVKLSDGRKFYAKTIVSNATRWDTFGKLLKAENLPKEEENFQKAYVKAPSFLSIHMGVKADVLPPDTDCHHFVLEDDWTNLEKPYGSIFLSIPTVLDSSLAPEGHHILHIFTTSSIEDWEGLSPKDYEAKKEVVAERIISRLEKTLFPGLKSSILFKEVGTPKTHRRYLARDSGTYGPMPRGTPKGLLGMPFNTTAIDGLYCVGDSCFPGQGVIAVAFSGVMCAHRVAADLGFEKKSDVLDSALLRLLGWLRTLA

SEQ ID NO: 43 - DNA, 1233 bp, Tagetes erecta
  CDS (1)..(1233)
atggccacacacaaactccttcaattcaccaccaatctcccaccatcttcttcttcaatctctactggctgttcactctcccccttcttcctcaaatcatcttctcattcccctaaccctcgccgacaccgccgctccgccgtatgctgctctttcgcctcactcgactctgcaaaaatcaaagtcgttggcgtcggtggtggtggcaacaatgccgttaaccgcatgattggtagcggcttacagggtgttgatttttacgccattaacacggactcacaagcgcttctgcaatctgttgcacataaccctattcaaattggggagcttttgactcgtggattaggtactggtgggaacccgcttttgggagaacaggctgcggaggagtcgaaggaagcgattgggaatgcgcttaaagggtcggatcttgtgtttataacagcaggtatgggtggtgggacgggttcgggtgctgctccagttgtagcgcagatagcgaaagaagcagggtatttaactgttggtgttgtaacgtacccattcagctttgaaggccgtaaaagatcagtacaggcgttagaggctattgagaagctgcaaaagaacgttgacacacttatagtgattccaaatgaccgtttgctggatattgctgatgaaaacacgcctcttcaggatgcttttcttcttgctgatgatgtactccgccaaggagttcaaggaatctcagatataattacaatacctgggctggtaaatgtggactttgcagacgttaaagcagtcatgaaagattctggaactgcaatgcttggtgtcggtgtttcctcaagtaaaaaccgagctgaagaagcagctgaacaagcaactcttgctcctttgattggatcatcaattcaatctgctacaggtgttgtttataatattaccggagggaaggacataactctacaagaagtcaacagggtttctcaggtggtaacaagtttggcagatccatcagcaaacattatattcggggcagtggtagatgagagatacaacggggagattcatgtgaccattgttgctactggctttgcccagtcgtttcagaaatctcttcttgctgacccgaaaggagcaaaacttgttgatagaaatcaagaacctacacaacctttgacttccgcgagatctttgacaacaccttctcctgctccgtctcggtctaggaaactcttcttttaa

SEQ ID NO: 44 - protein, 410 aa, Tagetes erecta
MATHKLLQFTTNLPPSSSSISTGCSLSPFFLKSSSHSPNPRRHRRSAVCCSFASLDSAKIKVVGVGGGGNNAVNRMIGSGLQGVDFYAINTDSQALLQSVAHNPIQIGELLTRGLGTGGNPLLGEQAAEESKEAIGNALKGSDLVFITAGMGGGTGSGAAPVVAQIAKEAGYLTVGVVTYPFSFEGRKRSVQALEAIEKLQKNVDTLIVIPNDRLLDIADENTPLQDAFLLADDVLRQGVQGISDIITIPGLVNVDFADVKAVMKDSGTAMLGVGVSSSKNRAEEAAEQATLAPLIGSSIQSATGVVYNITGGKDITLQEVNRVSQVVTSLADPSANIIFGAVVDERYNGEIHVTIVATGFAQSFQKSLLADPKGAKLVDRNQEPTQPLTSARSLTTPSPAPSRSRKLFF

SEQ ID NO: 45 - DNA, 891 bp, Tagetes erecta
  CDS (1)..(891)
atgacatccctgaggtttctaacagaaccctcacttgtatgctcatccactttccccacattcaatcccctacacaaaaccctaactaaaccaacaccaaaaccctacccaaagccaccaccaattcgctccgtccttcaatacaatcgcaaaccagagctcgccggagacactccacgagtcgtcgcaatcgacgccgacgttggtctacgtaacctcgatcttcttctcggtctcgaaaaccgcgtcaattacaccgtcgttgaagttctcaacggcgattgcagactcgaccaagccctagttcgtgataaacgctggtcaaatttcgaattgctttgtatttcaaaacctaggtcaaaattgcctttaggatttgggggaaaagctttagtttggcttgatgcattaaaagataggcaagaaggttgcccggattttatacttatagattgtcctgcaggtattgatgccgggttcataaccgccattacaccggctaacgaagccgtattagttacaacacctgatattactgcattgagagatgcagatagagttacaggcttgcttgaatgtgatggaattagggatattaaaatgattgtgaacagagttagaactgatttgataaggggtgaagatatgatgtcagttcttgatgttcaagagatgttgggattgtcattgttgagtgatacccgaggattcgaagtgattcggagtacgaatagagggtttccgcttgtgttgaacaagcctccgactttagcaggattggcatttgagcaggctgcttggagattggttgagcaagatagcatgaaggctgtgatggtggaggaagaacctaaaaagaggggatttttctcgttttttggaggttagtga

SEQ ID NO: 46 - protein, 295 aa, Tagetes erecta
MTSLRFLTEPSLVCSSTFPTFNPLHKTLTKPTPKPYPKPPPIRSVLQYNRKPELAGDTPRVVAIDADVGLRNLDLLLGLENRVNYTVVEVLNGDCRLDQALVRDKRWSNFELLCISKPRSKLPLGFGGKALVWLDALKDRQEGCPDFILIDCPAGIDAGFITAITPANEAVLVTTPDITALRDADRVTGLLECDGIRDIKMIVNRVRTDLIRGEDMMSVLDVQEMLGLSLLSDTRGFEVIRSTNRGFPLVLNKPPTLAGLAFEQAAWRLVEQDSMKAVMVEEEPKKRGFFSFFGG

SEQ ID NO: 47 - DNA, 1163 bp, Lycopersicon esculentum
  CDS (1)..(942)
attcggcacgagatttcagcctccgctagttcccgaaccattcgcctccgtcataacccgtttctcagtccaaaatccgcctcaaccgccccgccggttctgttcttctctccgttaactcgcaattttggcgcaattttgctgtctagaagaaagccgagattggcggtttgttttgtgctggagaatgagaaattgaatagtactatcgaaagtgagagtgaagtaatagaggatcggatacaagtagagattaatgaggagaagagtttagctgccagttggctggcggagaaattggcgaggaagaaatcggagaggtttacttatcttgtggcagctgtgatgtctagtttggggattacttctatggcgattttggcggtttattacagattttcatggcaaatggagggtggagaagtgcctttttctgaaatgttagctacattcactctctcgtttggcgctgccgtaggaatggagtactgggcgagatgggctcatagagcactatggcatgcttctttatggcacatgcacgagtcgcaccatagaccaagagaaggaccttttgagatgaacgacgttttcgccataacaaatgctgttccagctataggtcttctttcctacggtttcttccataaagggatcgtccctggcctctgtttcggcgctggattggggatcacagtatttgggatggcttacatgttcgttcacgatggactggttcataagagatttcccgtagggcctattgccaacgtgccttactttcggagggtagctgcagcacatcagcttcatcactcggacaaatttgatggtgtcccatatggcttgtttctaggacctaaggaattggaagaagtaggaggacttgaagagttagaaaaggaagtcaaccgaaggattaaaatttctaagggattattatgatcaaaagatacgtctgataataataaaatgcgattgtatttaggctgtagattattattgggaaaaagatagaaagatatatatatgaatataatataaaatgcaacaagctttctatggagaagaccttttcttttttggtacctgtacgtaaaaggtgaacaatttgatgtcctagtacttgttgacaaaccagaagaacgataattcaaaacaaa

SEQ ID NO: 48 - protein, 314 aa, Lycopersicon esculentum
IRHEISASASSRTIRLRHNPFLSPKSASTAPPVLFFSPLTRNFGAILLSRRKPRLAVCFVLENEKLNSTIESESEVIEDRIQVEINEEKSLAASWLAEKLARKKSERFTYLVAAVMSSLGITSMAILAVYYRFSWQMEGGEVPFSEMLATFTLSFGAAVGMEYWARWAHRALWHASLWHMHESHHRPREGPFEMNDVFAITNAVPAIGLLSYGFFHKGIVPGLCFGAGLGITVFGMAYMFVHDGLVHKRFPVGPIANVPYFRRVAAAHQLHHSDKFDGVPYGLFLGPKELEEVGGLEELEKEVNRRIKISKGLL

SEQ ID NO: 49 - DNA, 1666 bp, Lycopersicon esculentum
  CDS (1)..(1494)
atggaagctcttctcaagccttttccatctcttttactttcctctcctacaccccataggtctattttccaacaaaatccctcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcgaatcgggctcaattcgacgtgatcattatcggagctggccctgctgggctcaggctagctgaacaagtttctaaatatggtattaaggtatgttgtgttgacccttcaccactctccatgtggccaaataattatggtgtttgggttgatgagtttgagaatttaggactggaaaattgtttagatcataaatggcctatgacttgtgtgcatataaatgataacaaaactaagtatttgggaagaccatatggtagagttagtagaaagaagctgaagttgaaattgttgaatagttgtgttgagaacagagtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaggccaagaaaccatggttatcaaattgctcatggggttttagtagaagttgataatcatccatttgatttggataaaatggtgcttatggattggagggattctcatttgggtaatgagccatatttaagggtgaataatgctaaagaaccaacattcttgtatgcaatgccatttgatagagatttggttttcttggaagagacttctttggtgagtcgtcctgttttatcgtatatggaagtaaaaagaaggatggtggcaagattaaggcatttggggatcaaagtgaaaagtgttattgaggaagagaaatgtgtgatccctatgggaggaccacttccgcggattcctcaaaatgttatggctattggtgggaattcagggatagttcatccatcaacagggtacatggtggctaggagcatggctttagcaccagtactagctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgacgctttctttgatcttgatcctaaatactggcaagggttcctttcttcaagattgtctgtcaaagaacttggtttactcagcttgtgtcttttcggacatggctcaaacatgactaggttggatattgttacaaaatgtcctcttcctttggttagactgattggcaatctagcaatagagagcctttgaatgtgaaaagtttgaatcattttcttcattttaatttctttgattattttcatattttctcaattgcaaaagtgagataagagctacatactgtcaacaaataaactactattggaaagttaaaatatgtgtttgttgtatgttattctaatggaatggattttgtaaa

SEQ ID NO: 50 - protein, 498 aa, Lycopersicon esculentum
MEALLKPFPSLLLSSPTPHRSIFQQNPSFLSPTTKKKSRKCLLRNKSSKLFCSFLDLAPTSKPESLDVNISWVDPNSNRAQFDVIIIGAGPAGLRLAEQVSKYGIKVCCVDPSPLSMWPNNYGVWVDEFENLGLENCLDHKWPMTCVHINDNKTKYLGRPYGRVSRKKLKLKLLNSCVENRVKFYKAKVWKVEHEEFESSIVCDDGKKIRGSLVVDASGFASDFIEYDRPRNHGYQIAHGVLVEVDNHPFDLDKMVLMDWRDSHLGNEPYLRVNNAKEPTFLYAMPFDRDLVFLEETSLVSRPVLSYMEVKRRMVARLRHLGIKVKSVIEEEKCVIPMGGPLPRIPQNVMAIGGNSGIVHPSTGYMVARSMALAPVLAEAIVEGLGSTRMIRGSQLYHRVWNGLWPLDRRCVRECYSFGMETLLKLDLKGTRRLFDAFFDLDPKYWQGFLSSRLSVKELGLLSLCLFGHGSNMTRLDIVTKCPLPLVRLIGNLAIESL

SEQ ID NO: 51 - DNA, 358 bp, Tagetes erecta
  Promoter (1)..(358): Sense Promoter
aagcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggccaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttgtcgac

SEQ ID NO: 52 - DNA, 445 bp, Tagetes erecta
  misc_feature (1)..(445): Sense Fragment
aagcttgcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtcgac

SEQ ID NO: 53 - DNA, 446 bp, Tagetes erecta
  misc_feature (1)..(446): Antisense Fragment
gaattcgcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttggatcct

SEQ ID NO: 54 - DNA, 393 bp, Tagetes erecta
  misc_feature (1)..(393): Sense Fragment
aagctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctgtcgac

SEQ ID NO: 55 - DNA, 397 bp, Tagetes erecta
  misc_feature (1)..(397): Antisense Fragment
gaattctctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctggatcc

SEQ ID NO: 56 - DNA, 358 bp, Tagetes erecta; identical to SEQ ID NO: 51
  Promoter (1)..(358): Sense Promoter

SEQ ID NO: 57 - DNA, 361 bp, Tagetes erecta
  misc_feature (1)..(361): Antisense Promoter
ctcgagcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggccaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttggatcc

SEQ ID NO: 58 - DNA, 1830 bp, Tagetes erecta
  misc_feature (141)..(1691)
ggcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtaattggttgtggtcctgctggccttgctcttgctggagaatcagccaagctaggcttgaatgtcgcacttatcggccctgatcttccttttacaaataactatggtgtttgggaggatgaatttataggtcttggacttgagggctgtattgaacatgtttggcgagatactgtagtatatcttgatgacaacgatcccattctcataggtcgtgcctatggacgagttagtcgtgatttacttcacgaggagttgttgactaggtgcatggagtcaggcgtttcatatctgagctccaaagtggaacggattactgaagctccaaatggcctaagtctcatagagtgtgaaggcaatatcacaattccatgcaggcttgctactgtcgcttctggagcagcttctggaaaacttttgcagtatgaacttggcggtccccgtgtttgcgttcaaacagcttatggtatagaggttgaggttgaaagcataccctatgatccaagcctaatggttttcatggattatagagactacaccaaacataaatctcaatcactagaagcacaatatccaacatttttgtatgtcatgccaatgtctccaactaaagtattctttgaggaaacttgtttggcttcaaaagaggccatgccttttgagttattgaagacaaaactcatgtcaagattaaagactatggggatccgaataaccaaaacttatgaagaggaatggtcatatattccagtaggtggatccttaccaaataccgagcaaaagaaccttgcatttggtgctgctgctagcatggtgcatccagccacaggatattcggttgtaagatcactgtcagaagctcctaattatgcagcagtaattgcaaagattttagggaaaggaaattcaaaacagatgcttgatcatggaagatacacaaccaacatctcaaagcaagcttgggaaacactttggccccttgaaaggaaaagacagagagcattctttctctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctgg

SEQ ID NO: 59 - DNA, 37 bp, Artificial Sequence
  primer_bind (1)..(37): Primer
gcgcatgcatctagaaatgatccagttagaacaacca

SEQ ID NO: 60 - DNA, 37 bp, Artificial Sequence
  misc_feature (1)..(37): Primer
gcgcatgctctagactattttgctttgtaaatttctg

SEQ ID NO: 61 - DNA, 792 bp, Nostoc punctiforme
  CDS (5)..(766)
gcgcatgcatctagaaatgatccagttagaacaaccactgaaccatcaaacaaaactgaccccattagtaaaaaataaatctgcttttaggggcatttttattgctattgtcattattagtgtatggactattagcgagattttcttactttcgcttgatatctccaaattaaatatttggatgttatccgctttaatactttggcaaacatttttatatacgggattatttattacatctcatgatgctatgcatggggtagtgtttcccaaaaatcctaagattaatcgttttattggaacgttgagcttatctctttatggtcttttagcatatcaaaaactattgaaaaagcattggttacaccaccataatccagcgactgaaatagacccagatttccatgatggaaaacacaaaaatttcttcgcttggtattcttattttatgaagaactattggagttggggacaaatgattgccctaacttttatttatcactttgctagccacatccttcacataccgcatgaaaatctaatttcattttgggtttttccctcacttttaagttcattacagttattttattttggtacttatttaccccatagcgaaccaatagggggttatgttcaaccgcattgtgcagaaacgattagccgcccaatttggtggtcatttattacgtgctatcattttggctaccacaaagaacatcacgaatatcctcatgttccctggtggcagctcccagaaatttacaaagcaaaatagtctagagcatgcgc

SEQ ID NO: 62 - protein, 254 aa, Nostoc punctiforme
MHLEMIQLEQPLNHQTKLTPLVKNKSAFRGIFIAIVIISVWTISEIFLLSLDISKLNIWMLSALILWQTFLYTGLFITSHDAMHGVVFPKNPKINRFIGTLSLSLYGLLAYQKLLKKHWLHHHNPATEIDPDFHDGKHKNFFAWYSYFMKNYWSWGQMIALTFIYHFASHILHIPHENLISFWVFPSLLSSLQLFYFGTYLPHSEPIGGYVQPHCAETISRPIWWSFITCYHFGYHKEHHEYPHVPWWQLPEIY

SEQ ID NO: 63 - DNA, 32 bp, Artificial Sequence
  misc_feature (1)..(32): Primer
ggtaccttcattatttcgattttgatttcgtg

SEQ ID NO: 64 - DNA, 38 bp, Artificial Sequence
  misc_feature (1)..(38): Primer
aagcttggttgatcagaagaagaagaagaagatgaact

SEQ ID NO: 65 - DNA, 647 bp, Artificial Sequence
  misc_feature (1)..(647): Amplificate
ggtaccttcattatttcgattttgatttcgtgaccagcgaacgcagaataccttgttgtgtaatactttacccgtgtaaatcaaaaacaaaaaggcttttgagctttttgtagttgaatttctctggctgatcttttctgtacagattcatatatctgcagagacgatatcattgattatttgagcttcttttgaactatttcgtgtaatttgggatgagagctctatgtatgtgtgtaaactttgaagacaacaagaaaggtaacaagtgagggagggatgactccatgtcaaaatagatgtcataagaggcccatcaataagtgcttgagcccattagctagcccagtaactaccagattgtgagatggatgtgtgaacagttttttttttgatgtaggactgaaatgtgaacaacaggcgcatgaaaggctaaattaggacaatgataagcagaaataacttatcctctctaacacttggcctcacattgcccttcacacaatccacacacatccaatcacaacctcatcatatatctcccgctaatctttttttctttgatctttttttttttgcttattatttttttgactttgatctcccatcagttcatcttcttcttcttcttctgatcaaccaagctt

SEQ ID NO: 66 - DNA, 792 bp, Nostoc punctiforme
  CDS (5)..(766)
gcgcatgcatctagaaatgatccagttagaacaaccacccagttatcaaacaaaattgatttcaatagtgaaaagtaaatctcagtttaaaggacttttcattgctattgtcattgttagtgtatgggttattagcttgagtttattacttacccttgacatctcaaaattacaattttggatgttattgcctagcctagcttggcaaacatttttatacacgggattatttattacatctcatgatgctatgcatggggtagtatttccccaaaacagtaaaattaatcatcttattgggacattaaccctatctctttatggtcttttaccatataaaaaattattaaaaaagcattggttacaccaccaaaatccagcaactcaaatagacccagatttccataatggtaaacataaaaatttctttgcttggtatttccattttatgaagggttactggagttggggacaaattattgctctgacttttatctatcagtttgctaatcacatctttcatatacctcatgccaatctaataactttttgggtgcttccttcgcttttaagttcattccaattattttattttggtactttcctcccacatagtgaaccaatcgaaggctatattcagcctcattgtgcccaaactattagccgagcaattgggtggtcatttattacgtgctatcattttggctatcacgaagaacaccacgagtatcctcatattccttggtggcagttaccagaaatttacaaagcaaaatagtctagagcatgcgc

SEQ ID NO: 67 - protein, 254 aa, Nostoc punctiforme
MHLEMIQLEQPPSYQTKLISIVKSKSQFKGLFIAIVIVSVWVISLSLLLTLDISKLQFWMLLPSLAWQTFLYTGLFITSHDAMHGVVFPQNSKINHLIGTLTLSLYGLLPYKKLLKKHWLHHQNPATQIDPDFHNGKHKNFFAWYFHFMKGYWSWGQIIALTFIYQFANHIFHIPHANLITFWVLPSLLSSFQLFYFGTFLPHSEPIEGYIQPHCAQTISRAIGWSFITCYHFGYHEEHHEYPHIPWWQLPEIY

SEQ ID NO: 68 - DNA, 31 bp, Artificial Sequence
  misc_feature (1)..(31): Primer
gcgcatgctcacaaatttgatttagacattt

SEQ ID NO: 69 - DNA, 807 bp, Nodularia spumigena
  CDS (5)..(799)
gcgcatgcatctagaaatgatccagttagaacaaccaccattacctgaaattaaaatcactgctaccaccccagccgtgaaaagtaaatccctatttgggggcattttcatggcgatcgccattattagtatatgggctatcagcctcggtttgttactttatattgatatatcccaattcaagttttggatgttgttgccaatcatcttttggcaaacatttttatatacgggattatttattacagctcatgatgccatgcacggggtagtttttcccaaaaatcctaaaatcaaccatttcattggctcattgtgcttgtttctttatggtcttttaccttatcaaaaacttttaaaaaagcattggctacatcaccataatccagccagtgaaacagatccagattttcacaacggtaagcagaaaaacttttttgcttggtatttatattttatgaagcgttactggagttggttacaaattatcacattaatgattatctataacgtggtaaaagctatatggcatcttcctgatgataatatgacttatttttgggtagttccctcaattttaagttctttacaattattttattttggaacttttttaccccatcgtgaacctgtagaaggttatcaagatcctcatcgttctcaaactattagccgtccaatttggtggtcattcataacttgttaccattttggttatcatcatgaacatcatgaataccctcatgttccttggtggcagttacctgaagtttatcaaatgtctaaatcaaatttgtgagcatgcgc

SEQ ID NO: 70 - protein, 264 aa, Nodularia spumigena
MHLEMIQLEQPPLPEIKITATTPAVKSKSLFGGIFMAIAIISIWAISLGLLLYIDISQFKFWMLLPIIFWQTFLYTGLFITAHDAMHGVVFPKNPKINHFIGSLCLFLYGLLPYQKLLKKHWLHHHNPASETDPDFHNGKQKNFFAWYLYFMKRYWSWLQIITLMIIYNVVKAIWHLPDDNMTYFWVVPSILSSLQLFYFGTFLPHREPVEGYQDPHRSQTISRPIWWSFITCYHFGYHHEHHEYPHVPWWQLPEVYQMSKSNL

SEQ ID NO: 71 - DNA, 807 bp, Nodularia spumigena
  CDS (5)..(799)
gcgcatgcatctagaaatgatccagttagaacaaccaccattacctgaaattaaaatcactgctaccaccccagccgtgaaaagtaaatccctatttgggggcattttcatggcgatcgccattattagtatatgggctatcagcctgggtttgttactttatattgatatatcccaattcaacttttggatgttgttgccaatcatattttggcaaacatttttatatacgggattatttattacagctcatgatgccatgcacggggtagtttttcccaaaaatcctaaaatcaaccatttcattggctcattgtgcttgtttctttatggtcttttaccttatcaaaaacttttaaaaaagcattggctacatcaccataatccagccagtgaagcagatccagattttcacaacggtaagcagaaaaacttttttgcttggtatttatattttatgaagcgttactggagttggttacaaattatcacattaatgattatctttaacgtggtaaaatatatatggcatcttcctgatgataatctgacttatttttgggtagtgccatcaattttaagttccttacaattattttattttgggactttcttaccccatcgtgaacctgtagaaggttataaagatcctcatcgttctcaaactattagccgtccaatttggtggtcattcataacttgttaccattttggttatcatcatgaacatcacgaatacccccatgttccttggtggcaattacctgaaatttatcaaatgtctaaatcaaatttgtgagcatgcgc

SEQ ID NO: 72 - protein, 264 aa, Nodularia spumigena
MHLEMIQLEQPPLPEIKITATTPAVKSKSLFGGIFMAIAIISIWAISLGLLLYIDISQFNFWMLLPIIFWQTFLYTGLFITAHDAMHGVVFPKNPKINHFIGSLCLFLYGLLPYQKLLKKHWLHHHNPASEADPDFHNGKQKNFFAWYLYFMKRYWSWLQIITLMIIFNVVKYIWHLPDDNLTYFWVVPSILSSLQLFYFGTFLPHREPVEGYKDPHRSQTISRPIWWSFITCYHFGYHHEHHEYPHVPWWQLPEIYQMSKSNL

SEQ ID NO: 73 - DNA, 22 bp, Artificial Sequence
  misc_feature (1)..(22): Primer
cggcatgcgtggctcggcagta

SEQ ID NO: 74 - DNA, 21 bp, Artificial Sequence
  misc_feature (1)..(21): Primer
cgatgcatgcctagcggcgcg

SEQ ID NO: 75 - DNA, 785 bp, Gloeobacter violaceus
  CDS (5)..(775)
cggcatgcgtggctcggcagtaaaggaacgtacttcgaagcggcttgccgaaggggttatcacccataagaacgattcttccggcctctggtgggctctggtgattatcggcctgtggatcttcagtttcgccgcagcgctgcgcttacctattggcgagttatcgctgcaggccgtcatcggcgtggtgatcctcagaacctttctgcacacaggtctatttatcactgcccacgacgcgatgcaccgaaccgtgtttcccgccaatcaccgcatcaacgattggcttggtaccgccgccgtcggtctgtacgcctttatgccctatcgcgaactactgattaaacatcagttgcaccaccgctttccagccaccggcaaagaccccgactaccacgacggcgaacatagcggcttctttcagtggtacttgaaattcatgaaggactatatggagagccggaacaccccgtttttgatcgcgggcatggccgtggtgttcggggtgtgcacttggctgatgggcgttccgctcgtcaacctggcgctgttctggttgttgccgctggtgctcagttccttgcaattgttctacttcggcacctacttgccccaccgacaacccgacggcggctaccgcaaccgtcaccgggccaccagcaaccgtctttcgagcttctggtcatttgtcagctgctatcacttcggctaccactgggagcaccacgaatacccgctcgttccctggcatcggctgcccgaggcgcgccgctaggcatgcatcg

SEQ ID NO: 76 - protein, 256 aa, Gloeobacter violaceus; identical to SEQ ID NO: 14

SEQ ID NO: 77 - DNA, 24 bp, Artificial Sequence
  misc_feature (1)..(24): Primer
aggtaccgcacggtctgccaatcc

SEQ ID NO: 78 - DNA, 26 bp, Artificial Sequence
  misc_feature (1)..(26): Primer
aagcttgacctgattatcagcacggt

SEQ ID NO: 79 - DNA, 4624 bp, Erwinia uredovora
  CDS (128)..(1267)
  misc_feature (1288)..(2766): miscellaneous feature
gtcgactttcagcagcgcatggcgaaaatccagacagcccttcgtttggcagggggcaccatggccgctgccgatatcattgagcaggttatgtgcaccggtcagcctgtcttaagtgggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaaagagcgactacatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctagcgccatgtctttcccggagcgtcgcctgaagttttgacaggggcggcgcatagaggaagccaaaagaaacacaaccttctttgcccctgacggcgtgatgcatacggtgcgccatatacaaccgtttgaggtagcccttgcgtggaatatagcggaatggccaacgttgatgcaccagcccgtcgtgcaccataaaatagagtaatccatacgccgtcatacctgcgccaatccactggagcggccacattcctgtactgcccagataaatcagcaggatcgataatgcagcaaaaaccacggcataaagatcgttaacttcaaacgcacctttacgcggttcatgatgtgaaagatgccatccccaaccccagccgtgcatgatgtatttgtgtgccagtgcagcaatcacttccatgccaatcacggtaacgaaaacgatcagggcattccaaatccacaacataatttctccggtagagacgtctggcagcaggcttaaggattcaattttaacagagattagccgatctggcggcgggaagggaaaaaggcgcgccagaaaggcgcgccagggatcagaagtcggctttcagaaccacacggtagttggctttacctgcacgaacatggtccagtgcatcgttgattttcgacatcgggaagtactccactgtcggcgcaatatctgtacggccagccagcttcagcagtgaacgcagctgcgcaggtgaaccggttgaagaacccgtcacggcgcggtcgcctaaaatcaggctgaaagccgggcacgtcaaacggcttcagtacggcacccacggtatggaacttaccgcgaggcgccagggccgcaaagtagggttgccagtcgagatcgacggcgaccgtgctgataatcaggtcaaactggcccgccaggctttttaaagctt

SEQ ID NO: 80 - protein, 380 aa, Erwinia uredovora
MQPHYDLILVGAGLANGLIALRLQQQQPDMRILLIDAAPQAGGNHTWSFHHDDLTESQHRWIAPLVVHHWPDYQVRFPTRRRKLNSGYFCITSQRFAEVLQRQFGPHLWMDTAVAEVNAESVRLKKGQVIGARAVIDGRGYAANSALSVGFQAFIGQEWRLSHPHGLSSPIIMDATVDQQNGYRFVYSLPLSPTRLLIEDTHYIDNATLDPECARQNICDYAAQQGWQLQTLLREEQGALPITLSGNADAFWQQRPLACSGLRAGLFHPTTGYSLPLAVAVADRLSALDVFTSASIHHAITHFARERWQQQGFFRMLNRMLFLAGPADSRWRVMQRFYGLPEDLIARFYAGKLTLTDRLRILSGKPPVPVLAALQAIMTT

SEQ ID NO: 81 - DNA, 32 bp, Artificial Sequence
  misc_feature (1)..(32): Primer
tttttctcgagcgataaacgctcacttggtta

SEQ ID NO: 82 - DNA, 32 bp, Artificial Sequence
  misc_feature (1)..(32): Primer
tttttgtcgacacgttatgctcacaaccccgg

SEQ ID NO: 83 - DNA, 679 bp, Escherichia coli
  CDS (87)..(632)
ctcgagcgataaacgctcacttggttaatcatttcactcttcaattatctataatgatgagtgatcagaattacatgtgagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataaaaaaaccccgacatttgccggggttgtgagcataacgtgtcgac

SEQ ID NO: 84 - protein, 182 aa, Escherichia coli
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 85 - DNA, 31 bp, Artificial Sequence
  misc_feature (1)..(31): Primer
tttttccatggtgaaggaggaaatagcgaaa

SEQ ID NO: 86 - DNA, 32 bp, Artificial Sequence
  misc_feature (1)..(32): Primer
tttttaagctttcacttttttcttgtaaccaa

SEQ ID NO: 87 - DNA, 962 bp, A. fulgidus
  CDS (3)..(962)
ccatggtgaaggaggaaatagcgaaaagggccgaaataatcaacaaagccattgaagagcttctgcccgaaagggagccgattggactctacaaagccgcaaggcatctgatcaaagcaggtggcaagaggctaaggcctgtaataagcctcttagcagtcgaagcccttgggaaagactacagaaagattatcccggctgctgtcagcattgaaacaatccacaacttcaccctcgtgcatgacgacataatggacagggacgagatgaggaggggagttccgacggtacacagggtttatggggaagcgacggccattttagcaggcgacacactctttgctgaagccttcaagctgctgacaaagtgcgatgttgagagcgagggaatcagaaaagctacagaaatgctttcggacgtttgcataaaaatatgcgaggggcagtactacgacatgagctttgagaaaaaggagagcgtttccgaggaggagtatctcaggatggtcgagctgaagaccggagtgctgattgcagcttctgcagcattacctgcggtgctttttggggagagcgaggaaattgtaaaggcgctgtgggactacggagttcttagcggtattggcttccagatccaggacgacctgcttgacctgactgaggagaccggaaaggactggggaagcgacctgcttaaagggaagaaaaccctgattgtcataaaggcgttcgaaaagggagtgaagctaaagacgtttggaaaggaaaaggcggacgtctctgagattagagatgatatcgaaaagttaagagagtgtggtgcgattgattacgctgccagcatggcaagaaagatggctgaagaggcgaaaagaaagctcgaagttctgcctgaaagcaaagccaaggaaacactgctggaacttaccgacttcttggttacaagaaaaaagtgaaagctt

SEQ ID NO: 88 - protein, 317 aa, A. fulgidus
MVKEEIAKRAEIINKAIEELLPEREPIGLYKAARHLIKAGGKRLRPVISLLAVEALGKDYRKIIPAAVSIETIHNFTLVHDDIMDRDEMRRGVPTVHRVYGEATAILAGDTLFAEAFKLLTKCDVESEGIRKATEMLSDVCIKICEGQYYDMSFEKKESVSEEEYLRMVELKTGVLIAASAALPAVLFGESEEIVKALWDYGVLSGIGFQIQDDLLDLTEETGKDWGSDLLKGKKTLIVIKAFEKGVKLKTFGKEKADVSEIRDDIEKLRECGAIDYAASMARKMAEEAKRKLEVLPESKAKETLLELTDFLVTRKK

SEQ ID NO: 89 - DNA, 258 bp, Artificial sequence
  misc_feature (1)..(258): Nucleic acid sequence from the cassette of the plastid transit peptide of the plastid transketolase from tobacco as KpnI/BamHI fragment with an ATG codon in the NcoI cleavage site
ggtaccatggcgtcttcttcttctctcactctctctcaagctatcctctctcgttctgtccctcgccatggctctgcctcttcttctcaactttccccttcttctctcactttttccggccttaaatccaatcccaatatcaccacctcccgccgccgtactccttcctccgccgccgccgccgccgtcgtaaggtcaccggcgattcgtgcctcagctgcaaccgaaaccatagagaaaactgagactgcgggatcc

SEQ ID NO: 90 - DNA, 260 bp, Artificial sequence
  misc_feature (1)..(258): Nucleic acid sequence from the cassette of the plastid transit peptide of the plastid transketolase from tobacco as KpnI/BamHI fragment with an ATG codon in the NcoI cleavage site
ggtaccatggcgtcttcttcttctctcactctctctcaagctatcctctctcgttctgtccctcgccatggctctgcctcttcttctcaactttccccttcttctctcactttttccggccttaaatccaatcccaatatcaccacctcccgccgccgtactccttcctccgccgccgccgccgccgtcgtaaggtcaccggcgattcgtgcctcagctgcaaccgaaaccatagagaaaactgagactgcgctggatcc

SEQ ID NO: 91 - DNA, 259 bp, Artificial sequence
  misc_feature (1)..(258): Nucleic acid sequence from the cassette of the plastid transit peptide of the plastid transketolase from tobacco as KpnI/BamHI fragment with an ATG codon in the NcoI cleavage site
ggtaccatggcgtcttcttcttctctcactctctctcaagctatcctctctcgttctgtccctcgccatggctctgcctcttcttctcaactttccccttcttctctcactttttccggccttaaatccaatcccaatatcaccacctcccgccgccgtactccttcctccgccgccgccgccgccgtcgtaaggtcaccggcgattcgtgcctcagctgcaaccgaaaccatagagaaaactgagactgcggggatcc

(19) United States
(12) Patent Application Publication
(10) Pub. No.: US 2008/0060096 A1
(43) Pub. Date: Mar. 6, 2008

(54) Novel Ketolases and Method for Producing Ketocarotinoids

(75) Inventors: Matt Sauer, Quedlinburg (DE); Christel Renate Schopfer, Quedlinburg (DE); Ralf Flachmann, Quedlinburg (DE); Karin Herbers, Quedlinburg (DE); Irene Kunze, Gatersleben (DE); Martin Klebsattel, Quedlinburg (DE); Thomas Luck, Neustadt (DE); Dirk Voeste, Limburgerhof (DE); Angelika-Maria Pfeiffer, Birkensheide (DE); Hendrik Tschoep, Dresden (DE)

Correspondence Address: CONNOLLY BOVE LODGE & HUTZ, LLP, P O BOX 2207, WILMINGTON, DE, 19899, US

(73) Assignee: SunGene GmbH, Gatersleben (DE)

(21) Appl. No.: 10/569,064

(22) PCT Filed: Jul. 31, 2004
(86) PCT No.: PCT/EP04/08625

§ 371 Date: Feb. 17, 2006

(30) Foreign Application Priority Data
Aug. 18, 2003 (EP) PCT/EP03/09101
Aug. 18, 2003 (EP) PCT/EP03/09102
Aug. 18, 2003 (EP) PCT/EP03/09105
Aug. 18, 2003 (EP) PCT/EP03/09106
Aug. 18, 2003 (EP) PCT/EP03/09107
Aug. 18, 2003 (EP) PCT/EP03/09109
Feb. 17, 2004 (DE) 102004007624.3

(51) Int. Cl. A01H 5/00; C12P 7/26

(52) U.S. Cl. 800/295; 435/148

(57) ABSTRACT

The present invention relates to a process for preparing ketocarotenoids by cultivating genetically modified, non-human organisms which have, by comparison with the wild type, a modified ketolase activity, to the genetically modified organisms, to the use thereof as human and animal foods and for preparing ketocarotenoid extracts, and to novel ketolases and nucleic acids encoding these ketolases.

The present invention relates to a process for preparing ketocarotenoids by cultivating genetically modified, non-human organisms which have, by comparison with the wild type, a modified ketolase activity, to the genetically modified organisms, to the use thereof as human and animal foods and for preparing ketocarotenoid extracts, and to novel ketolases and nucleic acids encoding these ketolases.

Carotenoids are synthesized de novo in bacteria, algae, fungi and plants. Ketocarotenoids, i.e. carotenoids which comprise at least one keto group, such as, for example, astaxanthin, canthaxanthin, echinenone, 3-hydroxyechinenone, 3′-hydroxyechinenone, adonirubin and adonixanthin are natural antioxidants and pigments which are produced by some algae and microorganisms as secondary metabolites.

Because of their coloring properties, ketocarotenoids and especially astaxanthin are used as pigmentation aids in livestock nutrition, especially in trout, salmon and shrimp farming.

Astaxanthin is prepared nowadays for the most part by chemical synthetic processes. Natural ketocarotenoids such as, for example, natural astaxanthin are nowadays obtained in small amounts in biotechnological processes by cultivating algae, for example Haematococcus pluvialis, or by fermentation of genetically optimized microorganisms and subsequent isolation.

An economic biotechnological process for preparing natural ketocarotenoids is therefore of great importance.

Nucleic acids encoding a ketolase and the corresponding protein sequences have been isolated from various organisms and annotated, such as, for example, nucleic acids encoding a ketolase from Agrobacterium aurantiacum (EP 735 137, Accession NO: D58420), from Alcaligenes sp. PC-1 (EP 735137, Accession NO: D58422), Haematococcus pluvialis Flotow em. Wille and Haematoccus pluvialis, NIES-144 (EP 725137, WO 98/18910 and Lotan et al, FEBS Letters 1995, 364, 125-128, Accession NO: X86782 and D45881), Paracoccus marcusii (Accession NO: Y15112), Synechocystis sp. Strain PC6803 (Accession NO: NP_{—}442-491), Bradyrhizobium sp. (Accession NO: AF218415) and Nostoc sp. PCC 7120 (Kaneko et al, DNA Res. 2001, 8(5), 205-213; Accession NO: AP003592, BAB74888).

EP 735 137 describes the preparation of xanthophylls in microorganisms such as, for example, E. coli by introducing ketolase genes (crtW) from Agrobacterium aurantiacum or Alcaligenes sp. PC-1 into microorganisms.

EP 725 137, WO 98/18910, Kajiwara et al. (Plant Mol. Biol. 1995, 29, 343-352) and Hirschberg et al. (FEBS Letters 1995, 364, 125-128) disclose the preparation of astaxanthin by introducing ketolase genes from Haematococcus pluvialis (crtW, crtO or bkt) into E. coli.

Hirschberg et al. (FEBS Letters 1997, 404, 129-134) describe the preparation of astaxanthin in Synechococcus by introduction of ketolase genes (crtO) from Haematococcus pluvialis. Sandmann et al. (Photochemistry and Photobiology 2001, 73(5), 551-55) describe an analogous process which leads, however, to the preparation of canthaxanthin and affords only traces of astaxanthin.

WO 98/18910 and Hirschberg et al. (Nature Biotechnology 2000, 18(8), 888-892) describe the synthesis of ketocarotenoids in the nectaries of tobacco flowers by introducing the ketolase gene from Haematococcus pluvialis (crtO) into tobacco.

WO 01/20011 describes a DNA construct for producing ketocarotenoids, especially astaxanthin, in seeds of oilseed plants such as oilseed rape, sunflower, soybean and mustard using a seed-specific promoter and a ketolase from Haematococcus pluvialis.

All the ketolases and processes for preparing ketocarotenoids described in the prior art, and especially the processes described for preparing astaxanthin, have the disadvantage that, on the one hand, the yield is as yet unsatisfactory and, on the other hand, the transgenic organisms afford a large amount of hydroxylated by-products such as, for example, zeaxanthin and adonixanthin.

The invention was therefore based on the object of providing a process for preparing ketocarotenoids by cultivating genetically modified, non-human organisms, and to provide further genetically modified, non-human organisms which produce ketocarotenoids, and novel, advantageous ketolases, which exhibit the prior art disadvantages described above to a smaller extent or not at all or provide the desired ketocarotenoids, especially astaxanthin, in higher yields.

A process for preparing ketocarotenoids has accordingly been found, wherein genetically modified, non-human organisms which, by comparison with the wild type, have a modified ketolase activity are cultivated, and the modified ketolase activity is caused by a ketolase selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

A “ketolase activity which is modified by comparison with the wild type” means in the case where the initial organism or wild type has no ketolase activity preferably a “ketolase activity caused by comparison with the wild type”.

A “ketolase activity which is modified by comparison with the wild type” means in the case where the initial organism or wild type has a ketolase activity preferably a “ketolase activity raised by comparison with the wild type”.

The non-human organisms of the invention such as, for example, microorganisms or plants are preferably naturally able as initial organisms to produce carotenoids such as, for example, β-carotene or zeaxanthin, or can be made capable by genetic modification, such as, for example, rerouting of metabolic pathways or complementation, of producing carotenoids such as, for example, β-carotene or zeaxanthin.

Some organisms are already able as initial or wild-type organisms to produce ketocarotenoids such as, for example, astaxanthin or canthaxanthin. These organisms, such as, for example, Haematococcus pluvialis, Paracoccus marcusii, Xanthophyllomyces dendrorhous, Bacillus circulans, Chlorococcum, Phaffia rhodoyma, Adonisröschen (Adonis aestivalis), Neochloris wimmeri, Protosiphon botryoides, Scotiellopsis oocystiformis, Scenedesmus vacuolatus, Chlorela zoofingiensis, Ankistrodesmus braunii, Euglena sanguinea and Bacillus atrophaeus, exhibit a ketolase activity even as initial or wild-type organism.

The term “wild type” means according to the invention the corresponding initial organism.

Depending on the context, the term “organism” may mean the non-human initial organism (wild type) or a genetically modified non-human organism of the invention, or both.

Preferably, and especially in cases where unambiguous assignment of the plant or the wild type is not possible, “wild type” for the raising or causing of the ketolase activity, for the raising or causing, described below, of the hydroxylase activity, for the raising or causing, described below, of the β-cyclase-activity, for the raising, described below, of the HMG-CoA reductase activity, for the raising, described below, of the (E)-4-hydroxy-3-methylbut-2-enyl-diphosphate reductase activity, for the raising, described below, of the 1-deoxy-D-xylose-5-phosphate synthase activity, for the raising, described below, of the 1-deoxy-D-xylose-5-phosphate reductoisomerase activity, for the raising, described below, of the isopentenyl-diphosphate Δ-isomerase activity, for the raising, described below, of the geranyl-diphosphate synthase activity, for the raising, described below, of the farnesyl-diphosphate synthase activity, for the raising, described below, of the geranylgeranyl-diphosphate synthase activity, for the raising, described below, of the phytoene synthase activity, for the raising, described below, of the phytoene desaturase activity, for the raising, described below, of the zeta-carotene desaturase activity, for the raising, described below, of the crtISO activity, for the raising, described below, of the FtsZ activity, for the raising, described below, of the MinD activity, for the reduction, described below, of the ε-cyclase activity and for the reduction, described below, of the endogenous β-hydroxylase activity and the raising of the content of ketocarotenoids is in each case a reference organism.

This reference organism for microorganisms which exhibit a ketolase activity even as wild type is preferably Haematococcus pluvialis.

This reference organism for microorganisms which exhibit no ketolase activity as wild type is preferably blakeslea.

This reference organism for plants which exhibit a ketolase activity even as wild type is preferably Adonis aestivalis, Adonis flammeus or Adonis annuus, particularly preferably Adonis aestivalis.

This reference organism for plants which exhibit no ketolase activity in petals as wild type is preferably Tagetes erecta, Tagetes patula, Tagetes lucida, Tagetes pringlei, Tagetes palmeri, Tagetes minuta or Tagetes campanulata, particularly preferably Tagetes erecta.

Ketolase activity means the enzymic activity of a ketolase.

A ketolase means a protein which has the enzymatic activity of introducing a keto group on an optionally substituted β-ionone ring of carotenoids.

A ketolase means in particular a protein which has the enzymatic activity of converting β-carotene into canthaxanthin.

Accordingly, ketolase activity means the amount of β-carotene converted or amount of canthaxanthin formed in a particular time by the ketolase protein.

In one embodiment of the process of the invention, the initial organisms used are non-human organisms which exhibit a ketolase activity even as wild type or initial organism, such as, for example, Haematococcus pluvialis, Paracoccus marcusii, Xanthophyllomyces dendrorhous, Bacillus circulans, Chlorococcum, Phaffia rhodozyma, Adonisroschen (Adonis aestivalis), Neochloris wimmeri, Protosiphon botryoides, Scotiellopsis oocystiformis, Scenedesmus vacuolatus, Chlorela zoofingiensis, Ankistrodesmus braunii, Euglena sanguinea or Bacillus atrophaeus. In this embodiment, the effect of the genetic modification is to raise the ketolase activity by comparison with the wild type or initial organism.

When the ketolase activity is raised compared with the wild type, the amount of β-carotene converted or the amount of canthaxanthin formed in a particular time by the ketolase protein is raised by comparison with the wild type.

This raising of the ketolase activity preferably amounts to at least 5%, further preferably at least 20%, further preferably at least 50%, further preferably at least 100%, more preferably at least 300%, even more preferably at least 500%, especially at least 600% of the ketolase activity of the wild type.

The ketolase activity in genetically modified organisms of the invention and in wild-type and reference organisms is preferably determined under the following conditions:

The ketolase activity in plant or microorganism material is determined by a method based on that of Fraser et al., (J. Biol. Chem. 272(10): 6128-6135, 1997). The ketolase activity in plant or microorganism extracts is determined using the substrates β-carotene and canthaxanthin in the presence of lipid (soybean lecithin) and detergent (sodium cholate). Substrate/product ratios from the ketolase assays are ascertained by means of HPLC.

The ketolase activity can be raised in various ways, for example by switching off inhibitory regulatory mechanisms at the translation and protein level or by raising the gene expression of a nucleic acid encoding a ketolase compared with the wild type, for example by inducing the ketolase gene by activators or by introducing nucleic acids encoding a ketolase into the organism.

The raising of the gene expression of a nucleic acid encoding a ketolase means according to the invention in this embodiment also the manipulation of the expression of the organism's own endogenous ketolases. This can be achieved for example by modifying the promoter DNA sequence for ketolase-encoding genes. Such a modification, which results in a modified or, preferably, raised expression rate of at least one endogenous ketolase, can take place by deletion or insertion of DNA sequences.

It is possible as described above to modify the expression of at least one endogenous ketolase by applying exogenous stimuli. This can take place by particular physiological conditions, i.e. by applying foreign substances.

A further possibility for achieving raised expression of at least one endogenous ketolase gene is for a regulatory protein which is not present in the wild-type organism or is modified to interact with the promoter of these genes.

Such a regulator may be a chimeric protein which consists of a DNA binding domain and of a transcription activator domain, as described for example in WO 96/06166.

In a preferred embodiment, the raising of the ketolase activity compared with the wild type takes place by raising the gene expression of a nucleic acid encoding a ketolase selected from the group of
A ketolase comprising the amino acid sequence SEQ. ID. NO. 2, or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.
   is raised compared with the wild type.

In a further preferred embodiment, the raising of the gene expression of a nucleic acid encoding a ketolase takes place by introducing nucleic acids which encode ketolases selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.
   into the organism.

In this embodiment, the genetically modified organism of the invention accordingly has at least one exogenous (=heterologous) nucleic acid encoding a ketoase, or at least two endogenous nucleic acids encoding a ketolase, where the ketolases are selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

In another, preferred embodiment of the process of the invention, the starting organisms used are non-human organisms which exhibit no ketolase activity as wild type, such as, for example, blakeslea, marigold, Tagetes erecta, Tagetes lucida, Tagetes minuta, Tagetes pringlei, Tagetes palmeri and Tagetes campanulata.

In this preferred embodiment, the genetic modification causes the ketolase activity in the organisms. The genetically modified organism of the invention thus exhibits in this preferred embodiment a ketolase activity by comparison with the genetically unmodified wild-type, and is thus preferably capable of transgenic expression of a ketolase, where the ketolases are selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

In this preferred embodiment, the gene expression of a nucleic acid encoding a ketolase is caused, in analogy to the raising, described above, of the gene expression of a nucleic acid encoding a ketolase, preferably by introducing nucleic acids which encode ketolases into the initial organism, the ketolases are selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

It is possible in both embodiments to use for this in principle any ketolase gene, i.e. any nucleic acids which encodes a ketolase, where the ketolases are selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

All the nucleic acids mentioned in the description may be for example an RNA, DNA or cDNA sequence.

In the case of genomic ketolase sequences from eukaryotic sources which comprise introns and in the event that the host organism is unable or cannot be made able to express the corresponding ketolases, it is preferred to use nucleic acid sequences which have already been processed, such as the corresponding cDNAs.

Examples of nucleic acids encoding a ketolase, and the corresponding ketolases of group A, which can be used in the process of the invention are for example the ketolase sequences of the invention from

### Nodularia spumigena strain NSOR10,

nucleic acid: SEQ ID NO: 1, protein: SEQ ID NO: 2 (Acc. No.AY210783, incorrect sequence annotated as putative ketolase),

Nodularia spumigena (Culture Collection of Algae at the University of Vienna, (CCAUV) 01-037), nucleic acid: SEQ ID NO: 3, protein: SEQ ID NO: 4),
Nodularia spumigena (Culture Collection of Algae at the University of Vienna (CCAUV) 01-053), nucleic acid: SEQ ID NO: 5, protein: SEQ ID NO: 6) and
Nodularia spumigena (Culture Collection of Algae at the University of Vienna (CCAUV) 01-061), nucleic acid: SEQ ID NO: 7, protein: SEQ ID NO: 8)

An example of nucleic acids encoding a ketolase, and the corresponding ketolases of group B, which can be used in the process of the invention, are for example the ketolase sequences of the invention from

Nostoc puntiforme (Sammlung von Algenkulturen Göttingen (SAG) 60.79 nucleic acid: SEQ ID NO: 9, protein: SEQ ID NO: 10.

An example of nucleic acids encoding a ketolase, and the corresponding ketolases of group C, which can be used in the process of the invention are for example the ketolase sequences of the invention from

Nostoc puntiforme (Sammlung von Algenkulturen Göttingen (SAG) 71.79 nucleic acid: SEQ ID NO: 11, protein: SEQ ID NO: 12.

An example of nucleic acids encoding a ketolase, and the corresponding ketolases of group D, which can be used in the process of the invention are for example the ketolase sequences of the invention from

Gloeobacter violaceous PCC7421, Acc.Nr: GV7421 nucleic acid: SEQ ID NO: 13, protein: SEQ ID NO: 14.

Further natural examples of ketolases and ketolase genes which can be used in the process of the invention can easily be found for example in various organisms whose genomic sequence is known, by comparisons of identity of the amino acid sequences or of the corresponding back-translated nucleic acid sequences from databases with the sequences described above and especially with the sequences SEQ ID NO: 2 and/or 10 and/or 12 and/or 14.

Further natural examples of ketolases and ketolase genes can moreover easily be found starting from the nucleic acid sequences described above, especially starting from the sequences SEQ ID NO: 1 and/or 9 and/or 11 and/or 13 from various organisms whose genomic sequence is unknown, by hybridization techniques in a manner known per se.

The hybridization, and this condition applies to all nucleic acid sequences of the description, can take place under moderate (low stringency) or preferably under stringent (high stringency) conditions.

Such hybridization conditions, which apply to all nucleic acids in the description, are described for example in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

The conditions during the washing step can for example be selected from the range of conditions limited by those of low stringency (with 2×SSC at 50° C.) and those with high stringency (with 0.2×SSC at 50° C., preferably at 65° C.) (20×SSC: 0.3 M sodium citrate, 3 M sodium chloride, pH 7.0).

It is additionally possible to raise the temperature during the washing step from moderate conditions at room temperature, 22° C., to stringent conditions at 65° C.

Both parameters, the salt concentration and temperature, may be varied at the same time, and it is also possible to keep one of the two parameters constant and vary only the other one. Denaturing agents such as, for example, formamide or SDS can also be employed during the hybridization. Hybridization in the presence of 50% formamide is preferably carried out at 42° C.

Some examples of conditions for hybridization and washing step are given below:

### (1) Hybridization conditions with for example

(i) 4×SSC at 65° C., or
(ii) 6×SSC at 45° C., or
(iii) 6×SSC at 68° C., 100 mg/ml denatured fish sperm DNA, or
(iv) 6×SSC, 0.5% SDS, 100 mg/ml denatured, fragmented salmon sperm DNA at 68° C., or
(v) 6×SSC, 0.5% SDS, 100 mg/ml denatured, fragmented salmon sperm DNA, 50% formamide at 42° C., or
(vi) 50% formamide, 4×SSC at 42° C., or
(vii) 50% (vol/vol) formamide, 0.1% bovine serum albumin, 0.1% Ficoll, 0.1% polyvinylpyrrolidone, 50 mM sodium phosphate buffer of pH 6.5, 750 mM NaCl, 75 mM sodium citrate at 42° C., or
(viii) 2× or 4×SSC at 50° C. (moderate conditions), or
(ix) 30 to 40% formamide, 2× or 4×SSC at 42_(moderate conditions).

### (2) Washing steps for 10 minutes in each case with for example

(i) 0.015 M NaCl/0.0015 M sodium citrate/0.1% SDS at 50° C., or
(ii) 0.1×SSC at 65° C., or
(iii) 0.1×SSC, 0.5% SDS at 68° C., or
(iv) 0.1×SSC, 0.5% SDS, 50% formamide at 42° C., or
(v) 0.2×SSC, 0.1% SDS at 42° C., or
(vi) 2×SSC at 65° C. (moderate conditions).

The ketolases of group A comprise the amino acid sequence SEQ. ID. NO. 2 or a sequence derived from this sequence by substitution, insertion or deletion of amino acids and having an identity of at least 80%, preferably at least 85%, more preferably at least 90%, more preferably at least 95%, more preferably at least 97%, particularly preferably at least 99% at the amino acid level with the sequence SEQ. ID. NO. 2.

The ketolases of group B comprise the amino acid sequence SEQ. ID. NO. 10 or a sequence derived from this sequence by substitution, insertion or deletion of amino acids and having an identity of at least 90%, more preferably at least 95%, more preferably at least 97%, particularly preferably at least 99% at the amino acid level with the sequence SEQ. ID. NO. 10.

The ketolases of group C comprise the amino acid sequence SEQ. ID. NO. 12 or a sequence derived from this sequence by substitution, insertion or deletion of amino acids and having an identity of at least 90%, more preferably at least 95%, more preferably at least 97%, particularly preferably at least 99% at the amino acid level with the sequence SEQ. ID. NO. 12.

The ketolases of group D comprise the amino acid sequence SEQ. ID. NO. 14 or a sequence derived from this sequence by substitution, insertion or deletion of amino acids and having an identity of at least 50%, more preferably at least 60%, more preferably at least 70%, more preferably at least 80%, more preferably at least 85%, more preferably at least 90%, more preferably at least 95%, more preferably at least 97%, particularly preferably: at least 99% at the amino acid level with the sequence SEQ. ID. NO. 14.

The following definitions and conditions for comparing the identity of proteins apply to all proteins in the description.

The term “substitution” means the replacement of one or more amino acids by one or more amino acids. So-called conservative exchanges are preferably carried out, where the replaced amino acid has a similar property to the original amino acid, for example replacement of Glu by Asp, Gln by Asn, Val by Ile, Leu by Ile, Ser by Thr.

Deletion is the replacement of one amino acid by a direct linkage. Preferred positions for deletions are the termini of the polypeptide and the connections between the individual protein domains.

Insertions are introductions of amino acids into the polypeptide chain, in which case there is formal replacement of a direct linkage by one or more amino acids. Identity between two proteins means the identity of the amino acids over the complete length of the protein in each case, in particular the identity calculated by comparison with the aid of the Vector NTI Suite 7.1 software from Informax (USA) using the clustal method (Higgins D G, Sharp P M. Fast and sensitive multiple sequence alignments on a microcomputer. Comput Appl. Biosci. 1989 April; 5(2):151-1), setting the following parameters:

Multiple alignment parameter:

### Gap opening penalty 10

### Gap extension penalty 10

### Gap separation penalty range 8

### Gap separation penalty off

% identity for alignment delay 40

### Residue specific gaps off

### Hydrophilic residue gap off

### Transition weighing 0

Pairwise alignment parameter:

### FAST algorithm on

### K-tuple size 1

### Gap penalty 3

### Window size 5

### Number of best diagonals 5

A protein having an identity of at least 80% at the amino acid level with a particular sequence accordingly means a protein which on comparison of its sequence with the particular sequence, in particular according to the above programming logarithm with the above set of parameters, has an identity of at least 80%.

A protein having for example an identity of at least 80% at the amino acid level with the sequence SEQ ID NO: 2 accordingly means a protein which on comparison of its sequence with the sequence SEQ ID NO: 2 in particular according to the above programming logarithm with the above set of parameters, has an identity of at least 80%.

A protein having for example an identity of at least 90% at the amino acid level with the sequence SEQ ID NO: 10 accordingly means a protein which on comparison of its sequence with the sequence SEQ ID NO: 10 in particular according to the above programming logarithm with the above set of parameters, has an identity of at least 90%.

A protein having for example an identity of at least 90% at the amino acid level with the sequence SEQ ID NO: 12 accordingly means a protein which on comparison of its sequence with the sequence SEQ ID NO: 12 in particular according to the above programming logarithm with the above set of parameters, has an identity of at least 90%.

A protein for example having an identity of at least 50% at the amino acid level with the sequence SEQ ID NO: 14 accordingly means a protein which on comparison of its sequence with the sequence SEQ ID NO: 14 in particular according to the above programming logarithm with the above set of parameters, has an identity of at least 50%.

Suitable nucleic acid sequences can be obtained for example by back-translation of the polypeptide sequence in accordance with the genetic code.

The codons used for this purpose are preferably those frequently used in accordance with the organism-specific codon usage. The codon usage can be easily ascertained by means of computer analyses of other, known genes of the relevant organisms.

In a particularly preferred embodiment, a nucleic acid comprising the sequence SEQ ID NO: 2 is introduced into the organism.

In a further particularly preferred embodiment, a nucleic acid comprising the sequence SEQ ID NO: 10 is introduced into the organism.

In a further particularly preferred embodiment, a nucleic acid comprising the sequence SEQ ID NO: 12 is introduced into the organism.

In a further particularly preferred embodiment, a nucleic acid comprising the sequence SEQ ID NO: 14 is introduced into the organism.

All the abovementioned ketolase genes can moreover be prepared in a manner known per se by chemical synthesis from the nucleotide units such as, for example, by fragment condensation of individual overlapping, complementary nucleic acid units of the double helix. Chemical synthesis of oligonucleotides is possible for example in a known manner by the phosphoamidite method (Voet, Voet, 2nd edition, Wiley Press New York, pp. 896-897). Addition of synthetic oligonucleotides and filling in of gaps with the aid of the Klenow fragment of DNA polymerase and ligation reactions, and general cloning methods, are described in Sambrook et al. (1989), Molecular cloning: A laboratory manual, Cold Spring Harbor Laboratory Press.

In a preferred embodiment there is cultivation of plants which, compared with the wild type, additionally have a raised or caused hydroxylase activity and/or β-cyclase activity.

A “β-cyclase activity which is modified by comparison with the wild type” means in the case where the initial organism or wild type has no β-cyclase activity, preferably a “β-cyclase activity caused by comparison with the wild type”.

A “β-cyclase activity which is modified by comparison with the wild type” means in the case where the initial organism or wild type has a β-cyclase activity, preferably a “β-cyclase activity raised by comparison with the wild type”.

A “hydroxylase activity which is modified by comparison with the wild type” means in the case where the initial organism or wild type has no hydroxylase activity, preferably a “hydroxylase activity caused by comparison with the wild type”.

A “hydroxylase activity which is modified by comparison with the wild type” means in the case where the initial organism or wild type has a hydroxylase activity, preferably a “hydroxylase activity raised by comparison with the wild type”.

Hydroxylase activity means the enzymic activity of a hydroxylase.

A hydroxylase means a protein which exhibits the enzymatic activity of introducing a hydroxy group on the optionally substituted, β-ionone ring of carotenoids.

A hydroxylase means in particular a protein which exhibits the enzymatic activity of converting β-carotene into zeaxanthin or canthaxanthin into astaxanthin.

Accordingly, hydroxylase activity means the amount of β-carotene or canthaxanthin converted or amount of zeaxanthin or astaxanthin formed in a particular time by the hydroxylase protein.

Thus, when the hydroxylase activity is raised compared with the wild-type, the amount of β-carotene or canthaxanthin converted or the amount of zeaxanthin or astaxanthin formed in a particular time by the hydroxylase protein is raised by comparison with the wild type.

This raising of the hydroxylase activity preferably amounts to at least 5%, further preferably at least 20%, further preferably at least 50%, further preferably at least 100%, more preferably at least 300%, even more preferably at least 500%, in particular at least 600% of the hydroxylase activity of the wild type.

β-Cyclase activity means the enzymic activity of a β-cyclase.

A β-cyclase means a protein which has the enzymatic activity of converting a terminal, linear residue of lycopene into a β-ionone ring.

A β-cyclase means in particular a protein which has the enzymatic activity of converting γ-carotene into β-carotene.

Accordingly, β-cyclase activity means the amount of γ-carotene converted or amount of β-carotene formed in a particular time by the β-cyclase protein.

Thus, when the β-cyclase activity is raised compared with the wild type, the amount of γ-carotene converted or the amount of β-carotene formed in a particular time by the β-cyclase protein is raised by comparison with the wild type.

This raising of the β-cyclase activity preferably amounts to at least 5%, further preferably at least 20%, further preferably at least 50%, further preferably at least 100%, more preferably at least 300%, even more preferably at least 500%, in particular at least 600% of the β-cyclase activity of the wild type.

The hydroxylase activity in genetically modified organisms of the invention and in wild-type and reference organisms is preferably determined under the following conditions:

The activity of the hydroxylase is determined in vitro according to Bouvier et al. (Biochim. Biophys. Acta 1391 (1998), 320-328). Ferredoxin, ferredoxin-NADP oxidoreductase, catalase, NADPH and beta-carotene with mono- and digalactosyl glycerides is added to a defined amount of organism extract.

The hydroxylase activity is particularly preferably determined under the following conditions of Bouvier, Keller, d'Harlingue and Camara (Xanthophyll biosynthesis: molecular and functional characterization of carotenoid hydroxylases from pepper fruits (Capsicum annuum L.; Biochim. Biophys. Acta 1391 (1998), 320-328):

The in vitro assay is carried out in a volume of 0.250 ml volume. The mixture comprises 50 mM potassium phosphate (pH 7.6), 0.025 mg of spinach ferredoxin, 0.5 units of spinach ferredoxin-NADP+ oxidoreductase, 0.25 mM NADPH, 0.010 mg of beta-carotene (emulsified in 0.1 mg of Tween 80), 0.05 mM of a mixture of mono- and digalactosyl glycerides (1:1), 1 unit of catalase, 0.2 mg of bovine serum albumin and organism extract in varying volume. The reaction mixture is incubated at 30° C. for 2 hours. The reaction products are extracted with organic solvent such as acetone or chloroform/methanol (2:1) and determined by HPLC.

The β-cyclase activity in genetically modified organisms of the invention and in wild-type and reference organisms is preferably determined under the following conditions:

The activity of β-cyclase is determined in vitro according to Fraser and Sandmann (Biochem. Biophys. Res. Comm. 185(1) (1992) 9-15). Potassium phosphate as buffer (pH 7.6), lycopene as substrate, paprica stromal protein, NADP+, NADPH and ATP are added to a defined amount of organism extract.

The β-cyclase activity is particularly preferably determined under the following conditions of Bouvier, d'Harlingue and Camara (Molecular Analysis of carotenoid cyclae inhibition; Arch. Biochem. Biophys. 346(1) (1997) 53-64):

The in vitro assay is carried out in a volume of 250 μl volume. The mixture comprises 50 mM potassium phosphate (pH 7.6), various amounts of plant extract, 20 nM lycopene, 250 μg of paprica chromoplast stromal protein, 0.2 mM NADP+, 0.2 mM NADPH and 1 mM ATP. NADP/NADPH and ATP are dissolved in 10 μl of ethanol with 1 mg of Tween 80 immediately before the addition to the incubation medium. After a reaction time of 60 minutes at 30 C, the reaction is stopped by adding chloroform/methanol (2:1). The reaction products extracted into chloroform are analyzed by HPLC.

An alternative assay with radioactive substrate is described in Fraser and Sandmann (Biochem. Biophys. Res. Comm. 185(1) (1992) 9-15).

The hydroxylase activity and/or β-cyclase activity can be raised in various ways, for example by switching off inhibitory regulatory mechanisms at the expression and protein level or by raising the gene expression of nucleic acids encoding a hydroxylase and/or of nucleic acids encoding a β-cyclase, compared with the wild type.

The gene expression of the nucleic acids encoding a hydroxylase can be raised and/or the gene expression of the nucleic acid encoding a β-cyclase can be raised compared with the wild type likewise in various ways, for example by inducing the hydroxylase gene and/or β-cyclase gene by activators or by introducing one or more hydroxylase gene copies and/or β-cyclase gene copies, i.e. by introducing at least one nucleic acid encoding a hydroxylase and/or at least one nucleic acid encoding a β-cyclase into the organism.

Raising the gene expression of a nucleic acid encoding a hydroxylase and/or β-cyclase also means according to the invention manipulation of the expression of the organism's own endogenous hydroxylase and/or β-cyclase.

This can be achieved for example by modifying the promoter DNA sequence for hydroxylases and/or β-cyclases encoding genes. Such a modification resulting in a raised expression rate of the gene can take place for example by deletion or insertion of DNA sequences.

It is, as described above, possible to modify the expression of the endogenous hydroxylase and/or β-cyclase by application of exogenous stimuli. This can take place by particular physiological conditions, i.e. by application of foreign substances.

A further possibility for achieving a modified or raised expression of an endogenous hydroxylase and/or β-cyclase gene is for a regulatory protein which does not occur in the untransformed organism to interact with the promoter of this gene.

Such a regulator may be a chimeric protein which consists of a DNA-binding domain and of a transcription activator domain as described, for example, in WO 96/06166.

In a preferred embodiment, the gene expression of a nucleic acid encoding a hydroxylase is raised, and/or the gene expression of a nucleic acid encoding a β-cyclase is raised, by introducing at least one nucleic acid encoding a hydroxylase and/or by introducing at least one nucleic acid encoding a β-cyclase into the organism.

It is possible to use for this purpose in principle any hydroxylase gene or any β-cyclase gene, i.e. any nucleic acid which encodes a hydroxylase and any nucleic acid which encodes a β-cyclase.

In the case of genomic hydroxylase or β-cyclase nucleic acid sequences from eukaryotic sources which comprise introns and in the event that the host organism is unable or cannot be made able to express, the corresponding hydroxylase or β-cyclase, it is preferred to use nucleic acid sequences which have already been processed, such as the corresponding cDNAs.

Examples of hydroxylase genes are nucleic acids encoding a hydroxylase from Haematococcus pluvialis, Accession AX038729, WO 0061764); (nucleic acid: SEQ ID NO: 15, protein: SEQ ID NO: 16), and encoding hydroxylases of the following Accession numbers:

|emb|CAB55626.1, CAA70427.1, CAA70888.1, CAB55625.1, AF499108_{—}1, AF315289_{—}1, AF296158_{—}1, AAC49443.1, NP_{—}194300.1, NP_{—}200070.1, MG10430.1, CAC06712.1, AAM88619.1, CAC95130.1, AAL80006.1, AF162276_{—}1, M053295.1, AAN85601.1, CRTZ_ERWHE, CRTZ_PANAN, BAB79605.1, CRTZ_ALCSP, CRTZ_AGRAU, CAB56060.1, ZP_{—}00094836.1, MC44852.1, BAC77670.1, NP_{—}745389.1, NP_{—}344225.1, NP_{—}849490.1, ZP_{—}00087019.1, NP_{—}503072.1, NP_{—}852012.1, NPP_{—}115929.1, ZP_{—}00013255.1

A particularly preferred hydroxylase is moreover tomato hydroxylase (nucleic acid: SEQ. ID. No. 47; protein: SEQ. ID. No. 48).

Examples of β-cyclase genes are nucleic acids encoding a β-cyclase from tomato (Accession X86452). (Nucleic acid: SEQ ID NO: 17, protein: SEQ ID NO: 18) and β-cyclases of the following Accession numbers:

| | |
|---|---|
| S66350 | lycopene beta-cyclase (EC 5.5.1.—) - tomato |
| CAA60119 | lycopene synthase [Capsicum annuum] |
| S66349 | lycopene beta-cyclase (EC 5.5.1.—) - common tobacco |
| CAA57386 | lycopene cyclase [Nicotiana tabacum] |
| AAM21152 | lycopene beta-cyclase [Citrus sinensis] |
| AAD38049 | lycopene cyclase [Citrus × paradisi] |
| AAN86060 | lycopene cyclase [Citrus unshiu] |
| AAF44700 | lycopene beta-cyclase [Citrus sinensis] |
| AAK07430 | lycopene beta-cyclase [Adonis palaestina] |
| AAG10429 | beta cyclase [Tagetes erecta] |
| AAA81880 | lycopene cyclase |
| AAB53337 | lycopene beta cyclase |
| AAL92175 | beta-lycopene cyclase [Sandersonia aurantiaca] |
| CAA67331 | lycopene cyclase [Narcissus pseudonarcissus] |
| AAM45381 | beta cyclase [Tagetes erecta] |
| AAO18661 | lycopene beta-cyclase [Zea mays] |
| AAG21133 | chromoplast-specific lycopene beta-cyclase [Lycopersicon esculentum] |
| AAF18989 | lycopene beta-cyclase [Daucus carota] |
| ZP_001140 | hypothetical protein [Prochlorococcus marinus str. MIT9313] |
| ZP_001050 | hypothetical protein [Prochlorococcus marinus subsp. pastoris str. CCMP1378] |
| ZP_001046 | hypothetical protein [Prochlorococcus marinus subsp. pastoris str. |
| | CCMP1378] |
| ZP_001134 | hypothetical protein [Prochlorococcus marinus str. MIT9313] |
| ZP_001150 | hypothetical protein [Synechococcus sp. WH 8102] |
| AAF10377 | lycopene cyclase [Deinococcus radiodurans] |
| BAA29250 | 393aa long hypothetical protein [Pyrococcus horikoshii] |
| BAC77673 | lycopene beta-monocyclase [marine bacterium P99-3] |
| AAL01999 | lycopene cyclase [Xanthobacter sp. Py2] |
| ZP_000190 | hypothetical protein [Chloroflexus aurantiacus] |
| ZP_000941 | hypothetical protein [Novosphingobium aromaticivorans] |
| AAF78200 | lycopene cyclase [Bradyrhizobium sp. ORS278] |
| BAB79602 | crtY [Pantoea agglomerans pv. milletiae] |
| CAA64855 | lycopene cyclase [Streptomyces griseus] |
| AAA21262 | dycopene cyclase [Pantoea agglomerans] |
| C37802 | crtY protein - Erwinia uredovora |
| BAB79602 | crtY [Pantoea agglomerans pv. milletiae] |
| AAA64980 | lycopene cyclase [Pantoea agglomerans] |
| AAC44851 | lycopene cyclase |
| BAA09593 | lycopene cyclase [Paracoccus sp. MBIC1143] |
| ZP_000941 | hypothetical protein [Novosphingobium aromaticivorans] |
| CAB56061 | lycopene beta-cyclase [Paracoccus marcusii] |
| BAA20275 | lycopene cyclase [Erythrobacter longus] |
| ZP_000570 | hypothetical protein [Thermobifida fusca] |
| ZP_000190 | hypothetical protein [Chloroflexus aurantiacus] |
| AAK07430 | lycopene beta-cyclase [Adonis palaestina] |
| CAA67331 | lycopene cyclase [Narcissus pseudonarcissus] |
| AAB53337 | lycopene beta cyclase |
| BAC77673 | lycopene beta-monocyclase [marine bacterium P99-3] |

A particularly preferred β-cyclase is moreover the chromoplast-specific β-cyclase from tomato (AAG21133) (nucleic acid: SEQ. ID. No. 49; protein: SEQ. ID. No. 50)

Thus, in this preferred embodiment, at least one further hydroxylase gene and/or β-cyclase gene is present in the preferred transgenic organisms of the invention compared with the wild type.

In this preferred embodiment, the genetically modified organism has for example at least one exogenous nucleic acid encoding a hydroxylase or at least two endogenous nucleic acids encoding a hydroxylase and/or at least one exogenous nucleic acid encoding a β-cyclase or at least two endogenous nucleic acids encoding a β-cyclase.

In the preferred embodiment described above, the hydroxylase genes preferably used are nucleic acids which encode proteins comprising the amino acid sequence SEQ ID NO: 16 or 48 or a sequence derived from these sequences by substitution, insertion or deletion of amino acids and having an identity of at least 30%, preferably at least 50%, more preferably at least 70%, even more preferably at least 90%, most preferably at least 95% at the amino acid level with the sequences SEQ. ID. NO: 16 or 48, and having the enzymatic property of a hydroxylase.

Further examples of hydroxylases and hydroxylase genes can easily be found for example in various organisms whose genomic sequence is known, as described above, by homology comparisons of the amino acid sequences or of the corresponding back-translated nucleic acid sequences from databases with the sequences SEQ. ID. NO: 16 or 48.

Further examples of hydroxylases and hydroxylase genes can moreover easily be found for example starting from the sequences SEQ ID NO: 15 or 47 in various organisms whose genomic sequence is unknown, as described above, by hybridization and PCR techniques in a manner known per se.

In a further particularly preferred embodiment, the hydroxylase activity is raised by introducing nucleic acids into organisms which encode proteins comprising the amino acid sequence of the hydroxylase of the sequence SEQ ID NO: 16 or 48.

Suitable nucleic acid sequences can be obtained for example by back-translation of the polypeptide sequence in accordance with the genetic code.

The codons preferably used for this purpose are those frequently used in accordance with the organism-specific codon usage. The codon usage can easily be ascertained by means of computer analyses of other, known genes of the relevant organisms.

In a particularly preferred embodiment, a nucleic acid comprising the sequence SEQ. ID. NO: 15 or 47 is introduced into the organism.

The β-cyclase genes preferably used in the preferred embodiment described above are nucleic acids which encode proteins comprising the amino acid sequence SEQ ID NO: 18 or 50 or a sequence derived from these sequences by substitution, insertion or deletion of amino acids and having an identity of at least 30%, preferably at least 50%, more preferably at least 70%, even more preferably at least 90%, most preferably at least 95% at the amino acid level with the respective sequences SEQ ID NO: 18 or 50, and having the enzymatic property of a β-cyclase.

Further examples of β-cyclases and β-cyclase genes can easily be found in a manner known per se for example in various organisms whose genomic sequence is known, as described above, by homology comparisons of the amino acids sequences or of the corresponding back-translated nucleic acid sequences from databases with SEQ ID NO: 18 or 50.

Further examples of β-cyclases and β-cyclase genes can moreover easily be found for example starting from the sequence SEQ ID NO: 17 or 49 in various organisms whose genomic sequence is unknown by hybridization and PCR techniques.

In a further particularly preferred embodiment, the β-cyclase activity is raised by introducing nucleic acids into organisms which encode proteins comprising the amino acid sequence of the β-cyclase of sequence SEQ. ID. NO: 18 or 50.

Suitable nucleic acid sequences can be obtained for example by back-translation of the polypeptide sequence in accordance with the genetic code.

The codons preferably used for this purpose are those frequently used in accordance with the organism-specific codon usage. The codon usage can easily be ascertained by means of computer analyses of other, known genes of the relevant organisms.

In a particularly preferred embodiment, a nucleic acid comprising the sequence SEQ. ID. NO: 17 or 49 is introduced into the organism.

All the abovementioned hydroxylase genes or β-cyclase genes can moreover be prepared in a manner known per se by chemical synthesis from the nucleotide units such as, for example, by fragment condensation of individual overlapping, complementary nucleic acid units of the double helix. Chemical synthesis of oligonucleotides is possible for example in a known manner by the phosphoamidite method (Voet, Voet, 2nd edition, Wiley Press New York, pp. 896-897). Addition of synthetic oligonucleotides and filling in of gaps with the aid of the Klenow fragment of DNA polymerase and ligation reactions, and general cloning methods, are described in Sambrook et al. (1989), Molecular cloning: A laboratory manual, Cold Spring Harbor Laboratory Press.

In a further preferred embodiment there is cultivation of genetically modified, non-human organisms which additionally have a raised activity, compared with the wild type, of at least one of the activities selected from the group of HMG-CoA reductase activity, (E)-4-hydroxy-3-methylbut-2-enyl-diphosphate reductase activity, 1-deoxy-D-xylose-5-phosphate synthase activity, 1-deoxy-D-xylose-5-phosphate reductoisomerase activity, isopentenyl-diphosphate Δ-isomerase activity, geranyl-diphosphate synthase activity, farnesyl-diphosphate synthase activity, geranylgeranyl-diphosphate synthase activity, phytoene synthase activity, phytoene desaturase activity, zeta-carotene desaturase activity, crtISO activity, FtsZ activity and MinD activity.

HMG-CoA reductase activity means the enzymic activity of an HMG-CoA reductase (3-hydroxy-3-methylglutaryl coenzyme A reductase).

An HMG-CoA reductase means a protein which has the enzymatic activity of converting 3-hydroxy-3-methyglutaryl coenzyme A into mevalonate.

Accordingly, an HMG-CoA reductase activity means the amount of 3-hydroxy-3-methylglutaryl coenzyme A converted or amount of mevalonate formed in a particular time by the HMG-CoA reductase protein.

Thus, when the HMG-CoA reductase activity is raised compared with the wild type, the amount of 3-hydroxy-3-methylglutaryl coenzyme A converted or the amount of mevalonate formed in a particular time by HMG-CoA reductase protein is raised by comparison with the wild type.

This raising of the HMG-CoA reductase activity preferably amounts to at least 5%, further preferably at least 20%, further preferably at least 50%, further preferably at least 100%, more preferably at least 300%, even more preferably at least 500%, in particular at least 600% of the HMG-CoA reductase activity of the wild type.

The HMG-CoA reductase activity in genetically modified organism of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

The HMG-CoA reductase activity can be measured in accordance with published descriptions (e.g. Schaller, Grausem, Benveniste, Chye, Tan, Song and Chua, Plant Physiol. 109 (1995), 761-770; Chappell, Wolf, Proulx, Cuellar and Saunders, Plant Physiol. 109 (1995) 1337-1343). Organism tissue can be homogenized and extracted in cold buffer (100 mM potassium phosphate (pH 7.0), 4 mM MgCl₂, 5 mM DTT). The homogenate is centrifuged at 10 000 g at 4 C for 15 minutes. The supernatant is then centrifuged again at 100 000 g for 45-60 minutes. The HMG-CoA reductase activity is determined in the supernatant and in the pellet of the microsomal fraction (after resuspension in 100 mM potassium phosphate (pH 7.0) and 50 mM DTT). Aliquots of the solution and of the suspension (the protein content of the suspension corresponds to about 1-10 ug) are incubated in 100 mM potassium phosphate buffer (pH 7.0 with 3 mM NADPH and 20 μM (¹⁴C)HMG-CoA (58 μCi/μM) ideally in a volume of 26 μl, at 30 C for 15-60 minutes. The reaction is terminated by adding 5 μl of mevalonate lactone (1 mg/ml) and 6 N HCl. After the addition, the mixture is incubated at room temperature for 15 minutes. The (¹⁴C)-mevalonate formed in the reaction is quantified by adding 125 μl of a saturated potassium phosphate solution (pH 6.0) and 300 μl of ethyl acetate. The mixture is thoroughly mixed and centrifuged. The radioactivity can be determined by scintillation measurement.

(E)-4-hydroxy-3-methylbut-2-enyl-diphosphate reductase activity, also referred to as IytB or IspH, means the enzymic activity of an (E)-4-hydroxy-3-methylbut-2-enyl-diphosphate reductase.

An (E)-4-hydroxy-3-methylbut-2-enyl-diphosphate reductase means a protein which has the enzymatic activity of converting (E)-4-hydroxy-3-methylbut-2-enyl diphosphate into isopentenyl diphosphate and dimethylallyl diphosphates.

Accordingly, (E)-4-hydroxy-3-methylbut-2-enyl-diphosphate reductase activity means the amount of (E)-4-hydroxy-3-methylbut-2-enyl diphosphate converted or amount of isopentenyl diphosphate and/or dimethylallyl diphosphate formed in a particular time by the (E)-4-hydroxy-3-methylbut-2-enyl-diphosphate reductase protein.

Thus, when the (E)-4-hydroxy-3-methylbut-2-enyl-diphosphate reductase activity is raised compared with the wild type, the amount of (E)-4-hydroxy-3-methylbut-2-enyl diphosphate converted or the amount of isopentenyl diphosphate and/or dimethylallyl diphosphate formed in a particular time by the (E)-4-hydroxy-3-methylbut-2-enyl-diphosphate reductase protein is raised by comparison with the wild type.

This raising of the (E)-4-hydroxy-3-methylbut-2-enyl-diphosphate reductase activity preferably amounts to at least 5%, further preferably at least 20%, further preferably at least 50%, further preferably at least 100%, more preferably at least 300%, even more preferably at least 500%, in particular at least 600% of the (E)-4-hydroxy-3-methylbut-2-enyl-diphosphate reductase activity of the wild type.

The (E)-4-hydroxy-3-methylbut-2-enyl-diphosphate reductase activity in genetically modified, non-human organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction. The (E)-4-hydroxy-3-methylbut-2-enyl-diphosphate reductase activity can be determined by an immunological detection. The preparation of specific antibodies has been described by Rohdich and colleagues (Rohdich, Hecht, Gärtner, Adam, Krieger, Amslinger, Arigoni, Bacher and Eisenreich: Studies on the nonmevalonate terpene biosynthetic pathway: metabolic role of IspH (LytB) protein, Natl. Acad. Natl. Sci. USA 99 (2002), 1158-1163). Altincicek and colleagues (Altincicek, Duin, Reichenberg, Hedderich, Kollas, Hintz, Wagner, Wiesner, Beck and Jomaa: LytB protein catalyzes the terminal step of the 2-C-methyl-D-erythritol-4-phosphate pathway of isoprenoid biosynthesis; FEBS Letters 532 (2002,) 437-440) describes an in vitro system for determining the catalytic activity, which system follows the reduction of (E)-4-hydroxy-3-methyl-but-2-enyl diphosphat to isopentenyl diphosphate and dimethylallyl diphosphate.

1-Deoxy-D-xylose-5-phosphate synthase activity means the enzymic activity of a 1-deoxy-D-xylose-5-phosphate synthase.

A 1-deoxy-D-xylose-5-phosphate synthase means a protein which has the enzymatic activity of converting hydroxyethyl-ThPP and glyceraldehyde 3-phosphate into 1-deoxy-D-xylose 5-phosphate.

Accordingly, 1-deoxy-D-xylose-5-phosphate synthase activity means the amount of hydroxyethyl-ThPP and/or glyceraldehyde 3-phosphate converted or amount of 1-deoxy-D-xylose 5-phosphate formed in a particular time by the 1-deoxy-D-xylose-5-phosphate synthase protein.

Thus, when the 1-deoxy-D-xylose-5-phosphate synthase activity is raised compared with the wild type, the amount of hydroxyethyl-ThPP and/or glyceraldehyde 3-phosphate converted or the amount of 1-deoxy-D-xylose 5-phosphate formed in a particular time by the 1-deoxy-D-xylose-5-phosphate synthase protein is raised by comparison with the wild type.

This raising of the 1-deoxy-D-xylose-5-phosphate synthase activity preferably amounts to at least 5%, further preferably at least 20%, further preferably at least 50%, further preferably at least 100%, more preferably at least 300%, even more preferably at least 500%, in particular at least 600% of the 1-deoxy-D-xylose-5-phosphate synthase activity of the wild type.

The 1-deoxy-D-xylose-5-phosphate synthase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

The reaction solution (50-200 ul) for determining the D-1-deoxyxylulose-5-phosphate synthase activity (DXS) consists of 100 mM Tris-HCl (pH 8.0), 3 mM MgCl₂, 3 mM MnCl₂, 3 mM ATP, 1 mM thiamine diphosphate, 0.1% Tween-60, 1 mM potassium fluoride, 30 uM (2-¹⁴C)-pyruvate (0.5 uCi), 0.6 mM DL-glyceraldehyd 3-phosphate. The organism extract is incubated in the reaction solution at 37 C for 1 to 2 hours. The reaction is then stopped by heating at 80 C for 3 minutes. After centrifugation at 13 000 revolutions/minute for 5 minutes, the supernatant is evaporated, and the residue is resuspended in 50 ul of methanol, loaded onto a TLC plate for thin-layer chromatography (Silica-Gel 60, Merck, Darmstadt) and fractionated in N-propyl alcohol/ethyl acetate/water (6:1:3; v/v/v). This separates radiolabeled D-1-deoxyxylulose 5-phosphate (or D-1-deoxyxylulose) from (2-¹⁴C)-pyruvate. Quantification takes place by means of a scintillation counter. The method has been described in Harker and Bramley (FEBS Letters 448 (1999) 115-119). As an alternative, a fluorometric assay for determining DXS synthase activity has been described by Querol and colleagues (Analytical Biochemistry 296 (2001) 101-105).

1-Deoxy-D-xylose-5-phosphate reductoisomerase activity means the enzymic activity of a 1-deoxy-D-xylose-5-phosphate reductoisomerase, also called 1-deoxy-D-xylulose-5-phosphate reductoisomerase.

A 1-deoxy-D-xylose-5-phosphate reductoisomerase means a protein which has the enzymatic activity of converting 1-deoxy-D-xylose 5-phosphate into 2-C-methyl-D-erythritol 4-phosphate.

Accordingly, 1-deoxy-D-xylose-5-phosphate reductoisomerase activity means the amount of 1-deoxy-D-xylose 5-phosphate converted or amount of 2-C-methyl-D-erythritol 4-phosphate formed in a particular time by the 1-deoxy-D-xylose-5-phosphate reductoisomerase protein.

Thus, when the 1-deoxy-D-xylose-5-phosphate reductoisomerase activity is raised compared with the wild type, the amount of 1-deoxy-D-xylose 5-phosphate converted or the amount of 2-C-methyl-D-erythritol 4-phosphate formed in a particular time by the 1-deoxy-D-xylose-5-phosphate reductoisomerase protein is raised by comparison with the wild type.

This raising of the 1-deoxy-D-xylose-5-phosphate reductoisomerase activity preferably amounts to at least 5%, further preferably at least 20%, further preferably at least 50%, further preferably at least 100%, more preferably at least 300%, even more preferably at least 500%, in particular at least 600% of the 1-deoxy-D-xylose-5-phosphate reductoisomerase activity of the wild type.

The 1-deoxy-D-xylose-5-phosphate reductoisomerase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

The activity of D-1-deoxyxylulose-5-phosphate reductoisomerase (DXR) is measured in a buffer composed of 100 mM Tris-HCl (pH 7,5), 1 mM MnCl₂, 0,3 mM NADPH and 0.3 mM 1-deoxy-D-xylulose 4-phosphate which can for example be synthesized enzymatically (Kuzuyama, Takahashi, Watanabe and Seto: Tetrahedon letters 39 (1998) 4509-4512). The reaction is started by adding the organism extract. The reaction volume may typically be 0.2 to 0.5 mL, and incubation takes place at 37 C for 30-60 minutes. During this time, the oxidation of NADPH is followed by photometry at 340 nm.

Isopentenyl-diphosphate Δ-isomerase activity means the enzymic activity of an isopentenyl-diphosphate Δ-isomerase.

An isopentenyl-diphosphate Δ-isomerase means a protein which has the enzymatic activity of converting isopentenyl diphosphate into dimethylallyl phosphate. Accordingly, isopentenyl-diphosphate Δ-isomerase activity means the amount of isopentenyl diphosphate converted or the amount of dimethylallyl phosphate formed in a particular time by the isopentenyl-diphosphate D-Δ-isomerase protein.

Thus, when the isopentenyl-diphosphate Δ-isomerase activity is raised compared with the wild type, the amount of isopentenyl diphosphate converted or the amount of dimethylallyl phosphate formed in a particular time by the isopentenyl-diphosphate Δ-isomerase protein is raised by comparison with the wild type.

This raising of the isopentenyl-diphosphate Δ-isomerase activity preferably amounts to at least 5%, further preferably at least 20%, further preferably at least 50%, further preferably at least 100%, more preferably at least 300%, even more preferably at least 500%, in particular at least 600% of the isopentenyl-diphosphate Δ-isomerase activity of the wild type.

The isopentenyl-diphosphate Δ-isomerase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

Determinations of the activity of isopentenyl-diphosphate isomerase (IPP isomerase) can be carried out by the method presented by Fraser and colleagues (Fraser, Romer, Shipton, Mills, Kiano, Misawa, Drake, Schuch and Bramley: Evaluation of transgenic tomato plants expressing an additional phytoene synthase in a fruit-specific manner; Proc. Natl. Acad. Sci. USA 99 (2002), 1092-1097, based on Fraser, Pinto, Holloway and Bramley, Plant Journal 24 (2000), 551-558). For enzyme measurements, incubations are carried out with 0.5 uCi of (1-¹⁴C)IPP (isopentenyl pyrophosphate) (56 mCi/mmol, Amersham plc) as substrate in 0.4 M Tris-HCl (pH 8.0) with 1 mM DTT, 4 mM MgCl₂, 6 mM Mn Cl₂, 3 mM ATP, 0.1% Tween 60, 1 mM potassium fluoride in a volume of about 150-500 αl. Extracts are mixed with buffer (e.g. in the ratio 1:1) and incubated at 28° C. for at least 5 hours. Then about 200 ul of methanol is added, and then acidic hydrolysis is carried out by adding concentrated hydrochloric acid (final concentration 25%) at 37 C for about 1 hour. This is followed by extraction twice (500 μl each time) with petroleum ether (mixed with 10% diethyl ether). The radioactivity in an aliquot of the hyperphase is determined by means of a scintillation counter. The specific enzymic activity can be determined with a short incubation of 5 minutes because short reaction times suppress the formation of by-products (see Lützow and Beyer: The isopentenyl-diphosphate Δ-isomerase and its relation to the phytoene synthase complex in daffodil chromoplasts; Biochim. Biophys. Acta 959 (1988), 118-126) α

Geranyl-diphosphate synthase activity means the enzymic activity of a geranyl-diphosphate synthase.

A geranyl-diphosphate synthase means a protein which has the enzymatic activity of converting isopentenyl diphosphate and dimethylallyl phosphate into geranyl diphosphate.

Accordingly, geranyl-diphosphate synthase activity means the amount of isopentenyl diphosphate and/or dimethyallyl phosphate converted or amount of geranyl diphosphate formed in a particular time by the geranyl-diphosphate synthase protein.

Thus, when the geranyl-diphosphate synthase activity is raised compared with the wild type, the amount of isopentenyl diphosphate and/or dimethylallyl phosphate converted or the amount of geranyl diphosphate formed in a particular time by the geranyl-diphosphate synthase protein is raised by comparison with the wild type.

This raising of the geranyl-diphosphate synthase activity preferably amounts to at least 5%, further preferably at least 20%, further preferably at least 50%, further preferably at least 100%, more preferably at least 300%, even more preferably at least 500%, in particular at least 600% of the geranyl-diphosphate synthase activity of the wild type.

The geranyl-diphosphate synthase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

The activity of the geranyl-diphosphate synthase (GPP synthase) can be determined in 50 mM Tris-HCl (pH 7.6), 10 mM MgCl₂, 5 mM MnCl₂, 2 mM DTT, 1 mM ATP, 0.2% Tween-20, 5 μM (¹⁴C)IPP and 50 μM DMAPP (dimethylallyl pyrophosphate) after addition of organism extract (according to Bouvier, Suire, d'Harlingue, Backhaus and Camara: Molecular cloning of geranyl diphosphate synthase and compartmentation of monoterpene synthesis in plant cells, Plant Journal 24 (2000) 241-252). After incubation for, for example, 2 hours at 37° C., the reaction products are dephosphyrylated (according to Koyama, Fuji and Ogura: Enzymatic hydrolysis of polyprenyl pyrophosphats, Methods Enzymol. 110 (1985), 153-155) and analyzed by thin-layer chromatography and measurement of the incorporated radioactivity (Dogbo, Bardat, Quennemet, and Camara: Metabolism of plastid terpenoids: In vitro inhibition of phytoene synthesis by phenethyl pyrophosphate derivates, FEBS Letters 219 (1987) 211-215).

Farnesyl-diphosphate synthase activity means the enzymic activity of a farnesyl-diphosphate synthase.

A farnesyl-diphosphate synthase means a protein which has the enzymatic activity of sequentially converting 2 molecules of isopentenyl diphosphate with dimethylallyl diphosphate and the resulting geranyl diphosphate into farnesyl diphosphate.

Accordingly, farnesyl-diphosphate synthase activity means the amount of dimethylallyl diphosphates and/or isopentenyl diphosphate converted or amount of farnesyl diphosphate formed in a particular time by the farnesyl-diphosphate synthase protein.

Thus, when the farnesyl-diphosphate synthase activity is raised compared with the wild type, the amount of dimethylallyl diphosphates and/or isopentenyl diphosphate converted or the amount of farnesyl diphosphate formed in a particular time by the farnesyl-diphosphate synthase protein is raised by comparison with the wild type.

This raising of the farnesyl-diphosphate synthase activity preferably amounts to at least 5%, further preferably at least 20%, further preferably at least 50%, further preferably at least 100%, more preferably at least 300%, even more preferably at least 500%, in particular at least 600% of the farnesyl-diphosphate synthase activity of the wild type.

The farnesyl-diphosphate synthase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

The activity of farnesyl-pyrophosphate synthase (FPP synthase) can be determined by a method of Joly and Edwards (Journal of Biological Chemistry 268 (1993), 26983-26989). According to this, the enzymic activity is measured in a buffer composed of 10 mM HEPES (pH 7.2), 1 mM MgCl₂, 1 mM dithiothreitol, 20 uM geranyl pyrophosphate and 40 μM (1-¹⁴C) isopentenyl pyrophosphate (4 Ci/mmol). The reaction mixture is incubated at 37° C.; the reaction is stopped by adding 2.5 N HCl (in 70% ethanol with 19 μg/ml farnesol). The reaction products are thus hydrolyzed by acid hydrolysis at 37 C within 30 minutes. The mixture is neutralized by adding 10% NaOH and is extracted with hexane. An aliquot of the hexane phase can be measured with a scintillation counter to determine the incorporated radioactivity.

Alternatively, after incubation of organism extract and radiolabeled IPP, the reaction products can be separated by thin-layer chromatography (Silica Gel SE60, Merck) in benzene/methanol (9:1). Radiolabeled products are eluted and the radioactivity is determined (according to Gaffe, Bru, Causse, Vidal, Stamitti-Bert, Carde and Gallusci: LEFPS1, a tomato farnesyl pyrophosphate gene highly expressed during early fruit development; Plant Physiology 123 (2000) 1351-1362).

Geranylgeranyl-diphosphate synthase activity means the enzymic activity of a geranylgeranyl-diphosphate synthase.

A geranylgeranyl-diphosphate synthase means a protein which has the enzymatic activity of converting farnesyl diphosphate and isopentenyl diphosphate into geranylgeranyl diphosphate.

Accordingly, geranylgeranyl-diphosphate synthase activity means the amount of farnesyl diphosphate and/or isopentenyl diphosphate converted or amount of geranylgeranyl diphosphate formed in a particular time by the geranylgeranyl-diphosphate synthase protein.

Thus, when the geranylgeranyl-diphosphate synthase activity is raised compared with the wild type, the amount of farnesyl diphosphate and/or isopentenyl diphosphate converted or the amount of geranylgeranyl diphosphate formed in a particular time by the geranylgeranyl-diphosphate synthase protein is raised by comparison with the wild type.

This raising of the geranylgeranyl-diphosphate synthase activity preferably amounts to at least 5%, further preferably at least 20%, further preferably at least 50%, further preferably at least 100%, more preferably at least 300%, even more preferably at least 500%, in particular at least 600% of the geranylgeranyl-piphosphate synthase activity of the wild type.

The geranylgeranyl-diphosphate synthase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3.2 mM DTT and 0.5 mM PMSF is added shortly before the extraction.

Activity measurements of geranylgeranyl-pyrophosphate synthase (GGPP synthase) can be determined by the method described by Dogbo and Camara (in Biochim. Biophys. Acta 920 (1987), 140-148: Purification of isopentenyl pyrophosphate isomerase and geranylgeranyl pyrophosphate synthase from Capsicum chromoplasts by affinity chromatography). For this purpose, organism extract is added to a buffer (50 mM Tris-HCl (pH 7,6), 2 mM MgCl₂, 1 mM MnCl₂, 2 mM dithiothreitol, (1-¹⁴C)IPP (0.1 uCi, 10 μM), 15 uM DMAPP, GPP or FPP) with a total volume of about 200 ul. The incubation can take place at 30 C for 1-2 hours (or longer). The reaction is by adding 0.5 ml of ethanol and 0.1 ml of 6N HCl. After incubation at 37° C. for 10 minutes, the reaction mixture is neutralized with 6N NaOH, mixed with 1 ml of water and extracted with 4 ml of diethyl ether. The amount of radioactivity is determined in an aliquot (e.g. 0.2 mL) of the ether phase by scintillation counting. Alternatively, the radiolabeled prenyl alcohols can be extracted after acid hydrolysis into ether and separated by HPLC (25 cm column of Spherisorb ODS-1, 5 um; elution with methanol/water (90:10; v/v) at a flow rate of 1 ml/min) and quantified by means of a radioactivity monitor (according to Wiedemann, Misawa and Sandmann: Purification and enzymatic characterization of the geranylgeranyl pyrophosphate synthase from Erwinia uredovora after expression in Escherichia coli; Archives Biochemistry and Biophysics 306 (1993), 152-157).

Phytoene synthase activity means the enzymic activity of a phytoene synthase.

In particular a phytoene synthase means a protein which has the enzymatic activity of converting geranylgeranyl diphosphate into phytoene.

Accordingly, phytoene synthase activity means the amount of geranylgeranyl diphosphate converted or amount of phytoene formed in a particular time by the phytoene synthase protein.

Thus, when the phytoene synthase activity is raised compared with the wild type, the amount of geranylgeranyl diphosphate converted or the amount of phytoene formed in a particular time by the phytoene synthase protein is raised by comparison with the wild type.

This raising of the phytoene synthase activity preferably amounts to at least 5%, further preferably at least 20%, further preferably at least 50%, further preferably at least 100%, more preferably at least 300%, even more preferably at least 500%, in particular at least 600% of the phytoene synthase activity of the wild type. The phytoene synthase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

Determinations of the activity of phytoene synthase (PSY) can be carried out by the method presented by Fraser and colleagues (Fraser, Romer, Shipton, Mills, Kiano, Misawa, Drake, Schuch and Bramley: Evaluation of transgenic tomato plants expressing an additional phytoene synthase in a fruit-specific manner; Proc. Natl. Acad. Sci. USA 99 (2002), 1092-1097, based on Fraser, Pinto, Holloway and Bramley, Plant Journal 24 (2000) 551-558). For enzyme measurements, incubations are carried out with (3H)geranylgeranyl pyrophosphate (15 mCi/mM, American Radiolabeled Chemicals, St. Louis) as substrate in 0.4 M Tris-HCl (pH 8.0) with 1 mM DTT, 4 mM MgCl₂, 6 mM Mn Cl₂, 3 mM ATP, 0.1% Tween 60, 1 mM potassium fluoride. Organism extracts are mixed with buffer, e.g. 295 ul of buffer with extract in a total volume of 500 ul. Incubation is carried out at 28 C for at least 5 hours. Phytoene is then extracted by shaking twice with chloroform (500 ul each time). The radiolabeled phytoene formed during the reaction is separated by thin-layer chromatography on silica plates in methanol/water (95:5; v/v). Phytoene can be identified on the silica plates in an iodine-enriched atmosphere (by heating a few iodine crystals). A phytoene standard serves as reference. The amount of radiolabeled product is determined by measurement in a scintillation counter. Alternatively, phytoene can also be quantified by HPLC provided with a radioactivity detector (Fraser, Albrecht and Sandmann: Development of high performance liquid chromatographic systems for the separation of radiolabeled carotenes and precursors formed in specific enzymatic reactions; J. Chromatogr. 645 (1993) 265-272).

Phytoene desaturase activity means the enzymic activity of a phytoene desaturase.

A phytoene desaturase means a protein which has the enzymatic activity of converting phytoene into phytofluene and/or phytofluene into ζ-carotene (zeta-carotene).

Accordingly, phytoene desaturase activity means the amount of phytoene or phytofluene converted or the amount of phytofluene or ζ-carotene formed in a particular time by the phytoene desaturase protein.

Thus, when the phytoene desaturase activity is raised compared with the wild type, the amount of phytoene or phytofluene converted or the amount of phytofluene or ζ-carotene formed in a particular time by the phytoene desaturase protein is raised by comparison with the wild type.

This raising of the phytoene desaturase activity preferably amounts to at least 5%, further preferably at least 20%, further preferably at least 50%, further preferably at least 100%, more preferably at least 300%, even more preferably at least 500%, in particular at least 600% of the phytoene desaturase activity of the wild type.

The phytoene desaturase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

The activity of phytoene desaturase (PDS) can be measured through the incorporation of radiolabeled (¹⁴C)-phytoene into unsaturated carotenes (according to Romer, Fraser, Kiano, Shipton, Misawa, Schuch and Bramley: Elevation of the provitamin A content of transgenic tomato plants; Nature Biotechnology 18 (2000) 666-669). Radiolabeled phytoene can be synthesized according to Fraser (Fraser, De la Rivas, Mackenzie, Bramley: Phycomyces blakesleanus CarB mutants: their use in assays of phytoene desaturase; Phytochemistry 30 (1991), 3971-3976). Membranes of plastids of the target tissue can be incubated with 100 mM MES buffer (pH 6.0) with 10 mM MgCl₂ and 1 mM dithiothreitol in a total volume of 1 mL. (¹⁴C)-Phytoene dissolved in acetone (about 100 000 disintegrations/minute for each incubation) is added, but the acetone concentration should not exceed 5% (v/v). This mixture is incubated with shaking in the dark at 28 C for about 6 to 7 hours. Thereafter, pigments are extracted three times with about 5 mL of petroleum ether (mixed with 10% diethyl ether) and separated and quantified by HPLC.

Alternatively, the activity of the phytoene desaturase can be measured by the method of Fraser et al. (Fraser, Misawa, Linden, Yamano, Kobayashi and Sandmann: Expression in Escherichia coli, purification, and reactivation of the recombinant Erwinia uredovora phytoene desaturase, Journal of Biological Chemistry 267 (1992), 19891-9895).

Zeta-carotene desaturase activity means the enzymic activity of a zeta-carotene desaturase.

A zeta-carotene desaturase means a protein which has the enzymatic activity of converting ζ-carotene into neurosporin and/or neurosporin into lycopene.

Accordingly, zeta-carotene desaturase activity means the amount of ζ-carotene or neurosporin converted or amount of neurosporin or lycopene formed in a particular time by the zeta-carotene desaturase protein.

Thus, when the zeta-carotene desaturase activity is raised compared with the wild type, the amount of ζ-carotene or neurosporin converted or the amount of neurosporin or lycopene formed in a particular time by the zeta-carotene desaturase protein is raised by comparison with the wild type.

This raising of the zeta-carotene desaturase activity preferably amounts to at least 5%, further preferably at least 20%, further preferably at least 50%, further preferably at least 100%, more preferably at least 300%, even more preferably at least 500%, in particular at least 600% of the zeta-carotene desaturase activity of the wild type.

The zeta-carotene desaturase activity in genetically modified organisms of the invention and in wild-type and reference organisms is preferably determined under the following conditions:

Frozen organism material is homogenized by vigorous grinding in liquid nitrogen and extracted with extraction buffer in a ratio of from 1:1 to 1:20. The particular ratio depends on the enzymic activities in the available organism material, so that determination and quantification of the enzymic activities is possible within the linear measurement range. The extraction buffer may typically consist of 50 mM HEPES-KOH (pH 7.4), 10 mM MgCl2, 10 mM KCl, 1 mM EDTA, 1 mM EGTA, 0.1% (v/v) Triton X-100, 2 mM ε-aminocaproic acid, 10% glycerol, 5 mM KHCO3. 2 mM DTT and 0.5 mM PMSF is added shortly before the extraction.

Analyses to determine the ζ-carotene desaturase (ZDS desaturase) can be carried out in 0.2 M potassium phosphate (pH 7.8, buffer volume about 1 ml). The method of analysis for this purpose has been published by Breitenbach and colleagues (Breitenbach, Kuntz, Takaichi and Sandmann: Catalytic properties of an expressed and purified higher plant type ζ-carotene desaturase from Capsicum annuum; European Journal of Biochemistry. 265(1):376-383, 1999 October). Each mixture for analysis comprises 3 mg of phosphytidylcholine suspended in 0.4 M potassium phosphate buffer (pH 7.8), 5 αg of ζ-carotene or neurosporin, 0.02% butylated hydroxytoluene, 10 ul of decylplastoquinone (1 mM methanolic stock solution) and organism extract. The volume of the organism extract must be adapted to the amount of ZDS desaturase activity present in order to make quantifications in a linear measurement range possible. Incubations typically take place at about 28° C. in the dark with vigorous shaking (200 revolutions/minute) for about 17 hours. Carotenoids are extracted by adding 4 ml of acetone and shaking at 50° C. for 10 minutes. The carotenoids are transferred from this mixture into a petroleum ether phase (with 10% diethyl ether). The diethyl ether/petroleum ether phase is evaporated under nitrogen, and the carotenoids are redissolved in 20 ul and separated and quantified by HPLC.

crtISO activity means the enzymic activity of a crtISO protein.

A crtISO protein means a protein which has the enzymatic activity of converting 7,9,7′,9′-tetra-cis-lycopene into all-trans-lycopene.

Accordingly, crtISO activity means the amount of 7,9,7′,9′-tetra-cis-lycopene converted or amount of all-trans-lycopene formed in a particular time by the crtISO protein.

Thus, when the crtISO activity is raised compared with the wild type, the amount of 7,9,7′,9′-tetra-cis-lycopene converted or the amount of all-trans-lycopene formed in a particular time by the crtISO protein is raised by comparison with the wild type. This raising of the crtISO activity is preferably at least 5%, further preferably at least 20%, further preferably at least 50%, further preferably at least 100%, more preferably at least 300%, even more preferably at least 500%, in particular at least 600% of the crtISO activity of the wild type.

FtsZ activity means the physiological activity of an FtsZ protein.

An FtsZ protein means a protein which has a promoting effect on cell division and plastid division and displays homologies to tubulin proteins.

MinD activity means the physiological activity of a MinD protein.

A MinD protein means a protein which has a multifunctional role in cell division. It is a membrane-associated ATPase and can show an oscillating movement from pole to pole within the cell.

It is further possible for raising the activity of enzymes of the non-mevalonate pathway to lead to a further raising of the desired ketocarotenoid final product. Examples thereof are 4-diphosphocytidyl-2-C-methyl-D-erythritol synthase, 4-diphosphocytidyl-2-C-methyl-D-erythritol kinase and 2-C-methyl-D-erythritol-2,4-cyclodiphoshate synthase. The activity of said enzymes can be raised by altering the gene expression of the corresponding genes. The altered concentrations of the relevant proteins can be detected routinely by means of antibodies and appropriate blotting techniques.

The raising of the HMG-CoA reductase activity and/or (E)-4-hydroxy-3-methylbut-2-enyl-diphosphate reductase activity and/or 1-deoxy-D-xylose-5-phosphate synthase activity and/or 1-deoxy-D-xylose-5-phosphate reductoisomerase activity and/or isopentenyl-diphosphate Δ-isomerase activity and/or geranyl-diphosphate synthase activity and/or farnesyl-diphosphate synthase activity and/or geranylgeranyl-diphosphate synthase activity and/or phytoene synthase activity and/or phytoene desaturase activity and/or zeta-carotene desaturase activity and/or crtISO activity and/or FtsZ activity and/or MinD activity can take place in various ways, for example by switching off inhibitory regulatory mechanisms at the expression and protein level or by raising gene expression of nucleic acids encoding an HMG-CoA reductase and/or nucleic acids encoding an (E)-4-hydroxy-3-methylbut-2-enyl-diphosphate reductase and/or nucleic acids encoding a 1-deoxy-D-xylose-5-phosphate synthase and/or nucleic acids encoding a 1-deoxy-D-xylose-5-phosphate reductoisomerase and/or nucleic acids encoding an isopentenyl-diphosphate Δ-isomerase and/or nucleic acids encoding a geranyl-diphosphate synthase and/or nucleic acids encoding a farnesyl-diphosphate synthase and/or nucleic acids encoding a geranylgeranyl-diphosphate synthase and/or nucleic acids encoding a phytoene synthase and/or nucleic acids encoding a phytoene desaturase and/or nucleic acids encoding a zeta-carotene desaturase and/or nucleic acids encoding a crtISO protein and/or nucleic acids encoding an FtsZ protein and/or nucleic acids encoding a MinD protein compared with the wild type.

The raising of the gene expression of nucleic acids encoding an HMG-CoA reductase and/or nucleic acids encoding an (E)-4-hydroxy-3-methylbut-2-enyl-diphosphate reductase and/or nucleic acids encoding a 1-deoxy-D-xylose-5-phosphate synthase and/or nucleic acids encoding a 1-deoxy-D-xylose-5-phosphate reductoisomerase and/or nucleic acids encoding an isopentenyl-diphosphate Δ-isomerase and/or nucleic acids encoding a geranyl-diphosphate synthase and/or nucleic acids encoding a farnesyl-diphosphate synthase and/or nucleic acids encoding a geranylgeranyl-diphosphate synthase and/or nucleic acids encoding a phytoene synthase and/or nucleic acids encoding a phytoene desaturase and/or nucleic acids encoding a zeta-carotene desaturase and/or nucleic acids encoding a crtISO protein and/or nucleic acids encoding an FtsZ protein and/or nucleic acids encoding a MinD protein compared with the wild type can likewise take place in various ways, for example by inducing the HMG-CoA reductase gene and/or (E)-4-hydroxy-3-methylbut-2-enyl-diphosphate reductase gene and/or 1-deoxy-D-xylose-5-phosphate synthase gene and/or 1-deoxy-D-xylose-5-phosphate reductoisomerase gene and/or isopentenyl-diphosphate Δ-isomerase gene and/or geranyl-diphosphate synthase gene and/or farnesyl-diphosphate synthase gene and/or geranylgeranyl-diphosphate synthase gene and/or phytoene synthase gene and/or phytoene desaturase gene and/or zeta-carotene desaturase gene and/or crtISO gene and/or FtsZ gene and/or MinD gene, by activators or by introducing one or more copies of the HMG-CoA reductase gene and/or (E)-4-hydroxy-3-methylbut-2-enyl-diphosphate reductase gene and/or 1-deoxy-D-xylose-5-phosphate synthase gene and/or 1-deoxy-D-xylose-5-phosphate reductoisomerase gene and/or isopentenyl-diphospate Δ-isomerase gene and/or geranyl-diphosphate synthase gene and/or farnesyl-diphosphate synthase gene and/or geranylgeranyl-diphosphate synthase gene and/or phytoene synthase gene and/or phytroene desaturase gene and/or zeta-carotene desaturase gene and/or crtISO gene and/or FtsZ gene and/or minD gene i.e. by introducing at least one nucleic acid encoding an HMG-CoA reductase and/or at least one nucleic acid encoding an (E)-4-hydroxy-3-methylbut-2-enyl-diphosphate reductase and/or at least one nucleic acid encoding a 1-deoxy-D-xylose-5-phosphate synthase and/or at least one nucleic acid encoding a 1-deoxy-D-xylose-5-phosphate reductoisomerase and/or at least one nucleic acid encoding an isopentenyl-diphosphate Δ-isomerase and/or at least one nucleic acid encoding a geranyl-diphosphate synthase and/or at least one nucleic acid encoding a farnesyl-diphosphate synthase and/or at least one nucleic acid encoding a geranylgeranyl-diphosphate synthase and/or at least one nucleic acid encoding a phytoene synthase and/or at least one nucleic acid encoding a phytoene desaturase and/or at least one nucleic acid encoding a zeta-carotene desaturase and/or at least one nucleic acid encoding a crtISO protein and/or at least one nucleic acid encoding an FtsZ protein and/or at least one nucleic acid encoding a MinD protein into the organism.

Raising the gene expression of a nucleic acid encoding an HMG-CoA reductase and/or (E)-4-hydroxy-3-methylbut-2-enyl-diphosphate reductase and/or 1-deoxy-D-xylose-5-phosphate synthase and/or 1-deoxy-D-xylose-5-phosphate reductoisomerase and/or isopentenyl-diphosphate Δ-isomerase and/or geranyl-diphosphate synthase and/or farnesyl-diphosphate synthase and/or geranylgeranyl-diphosphate synthase and/or phytoene synthase and/or phytoene desaturase and/or zeta-carotene desaturase and/or a crtISO protein and/or FtsZ protein and/or MinD protein means according to the invention also the manipulation of the expression of the organism's own endogenous HMG-CoA reductase and/or (E)-4-hydroxy-3-methylbut-2-enyl-diphosphate reductase and/or 1-deoxy-D-xylose-5-phosphate synthase and/or 1-deoxy-D-xylose-5-phosphate reductoisomerase and/or isopentenyl-diphosphate Δ-isomerase and/or geranyl-diphosphate synthase and/or farnesyl-diphosphate synthase and/or geranylgeranyl-diphosphate synthase and/or phytoene synthase and/or phytoene desaturase and/or zeta-carotene desaturase and/or of the organisms own crtISO protein and/or FtsZ protein and/or MinD protein.

This can be achieved for example by modifying the corresponding promoter DNA sequence. Such a modification resulting in a raised rate of gene expression can take place for example by deletion or insertion of DNA sequences.

In a preferred embodiment, the raising of the gene expression of a nucleic acid encoding an HMG-CoA reductase and/or the raising of the gene expression of a nucleic acid encoding an (E)-4-hydroxy-3-methylbut-2-enyl-diphosphate reductase and/or the raising of the gene expression of a nucleic acid encoding a 1-deoxy-D-xylose-5-phosphate synthase and/or the raising of the gene expression of a nucleic acid encoding a 1-deoxy-D-xylose-5-phosphate reductoisomerase and/or the raising of the gene expression of a nucleic acid encoding an isopentenyl-diphosphate Δ-isomerase and/or the raising of the gene expression of a nucleic acid encoding a geranyl-diphosphate synthase and/or the raising of the gene expression of a nucleic acid encoding a farnesyl-diphosphate synthase and/or the raising of the gene expression of a nucleic acid encoding a geranylgeranyl-diphosphate synthase and/or the raising of the gene expression of a nucleic acid encoding a phytoene synthase and/or the raising of the gene expression of a nucleic acid encoding a phytoene desaturase and/or the raising of the gene expression of a nucleic acid encoding a zeta-carotene desaturase and/or the raising of the gene expression of a nucleic acid encoding a crtISO protein and/or the raising of the gene expression of a nucleic acid encoding an FtsZ protein and/or the raising of the gene expression of a nucleic acid encoding a MinD protein is effected by introducing at least one nucleic acid encoding an HMG-CoA reductase and/or by introducing at least one nucleic acid encoding a (E)-4-hydroxy-3-methylbut-2-enyl-diphosphate reductase and/or by introducing at least one nucleic acid encoding a 1-deoxy-D-xylose-5-phosphate synthase and/or by introducing at least one nucleic acid encoding a 1-deoxy-D-xylose-5-phosphate reductoisomerase and/or by introducing at least one nucleic acid encoding an isopentenyl-diphosphate. Δ-isomerase and/or by introducing at least one nucleic acid encoding a geranyl-diphosphate synthase and/or by introducing at least one nucleic acid encoding a farnesyl-diphosphate synthase and/or by introducing at least one nucleic acid encoding a geranylgeranyl-diphosphate synthase and/or by introducing at least one nucleic acid encoding a phytoene synthase and/or by introducing at least one nucleic acid encoding a phytoene desaturase and/or by introducing at least one nucleic acid encoding a zeta-carotene desaturase and/or by introducing at least one nucleic acid encoding a crtISO protein and/or by introducing at least one nucleic acid encoding an FtsZ protein and/or by introducing at least one nucleic acid encoding a MinD protein into the organism. It is possible in principle to use for this purpose any HMG-CoA reductase gene or (E)-4-hydroxy-3-methylbut-2-enyl-diphosphate reductase gene or 1-deoxy-D-xylose-5-phosphate synthase gene or 1-deoxy-D-xylose-5-phosphate reductoisomerase gene or isopentenyl-diphosphate Δ-isomerase gene or geranyl-diphosphate synthase gene or farnesyl-diphosphate synthase gene or geranylgeranyl-diphosphate synthase gene or phytoene synthase gene or phytoene desaturase gene or zeta-carotene desaturase gene or crtISO gene or FtsZ gene or MinD gene.

In the case of genomic HMG-CoA reductase sequences or (E)-4-hydroxy-3-methylbut-2-enyl-diphosphate reductase sequences or 1-deoxy-D-xylose-5-phosphate synthase sequences or 1-deoxy-D-xylose-5-phosphate reductoisomerase sequences or isopentenyl-diphosphate Δ-isomerase sequences or geranyl-diphosphate synthase sequences or farnesyl-diphosphate synthase sequences or geranylgeranyl-diphosphate synthase sequences or phytoene synthase sequences or phytoene desaturase sequences or zeta-carotene desaturase sequences or crtISO sequences or FtsZ sequences or MinD sequences from eukaryotic sources which comprise introns, in the event that the host organism is unable or cannot be made able to express the corresponding proteins, it is preferred to use already processed nucleic acid sequences such as the corresponding cDNAs.

Thus, in this preferred embodiment, in the preferred transgenic organisms of the invention there is present compared with the wild type at least one further HMG-CoA reductase gene and/or (E)-4-hydroxy-3-methylbut-2-enyl-diphosphate reductase gene and/or 1-deoxy-D-xylose-5-phosphate synthase gene and/or 1-deoxy-D-xylose-5-phosphate reductoisomerase gene and/or isopentenyl-diphosphate Δ-isomerase gene and/or geranyl-diphosphate synthase gene and/or farnesyl-diphosphate synthase gene and/or geranylgeranyl-diphosphate synthase gene and/or phytoene synthase gene and/or phytoene desaturase gene and/or zeta-carotene desaturase gene and/or crtISO gene and/or FtsZ gene and/or MinD gene.

In this preferred embodiment, the genetically modified organism has for example at least one exogenous nucleic acid encoding an HMG-CoA reductase or at least two endogenous nucleic acids, encoding an HMG-CoA reductase and/or at least one exogenous nucleic acid encoding an (E)-4-hydroxy-3-methylbut-2-enyl-diphosphate reductase or at least two endogenous nucleic acids encoding an (E)-4-hydroxy-3-methylbut-2-enyl-diphosphate reductase and/or at least one exogenous nucleic acid-encoding a 1-deoxy-D-xylose-5-phosphate synthase or at least two endogenous nucleic acids encoding a 1-deoxy-D-xylose-5-phosphate synthase and/or at least one exogenous nucleic acid encoding a 1-deoxy-D-xylose-5-phosphate reductoisomerase or at least two endogenous nucleic acids encoding a 1-deoxy-D-xylose-5-phosphate reductoisomerase and/or at least one exogenous nucleic acid encoding an isopentenyl-diphosphate Δ-isomerase or at least two endogenous nucleic acids encoding an isopentenyl-diphosphate Δ-isomerase and/or at least one exogenous nucleic acid encoding a geranyl-diphosphate synthase or at least two endogenous nucleic acids encoding a geranyl-diphosphate synthase and/or at least one exogenous nucleic acid encoding a farnesyl-diphosphate synthase or at least two endogenous nucleic acids encoding a farnesyl-diphosphate synthase and/or at least one exogenous nucleic acid encoding a geranylgeranyl-diphosphate synthase or at least two endogenous nucleic acids encoding a geranylgeranyl-diphosphate synthase and/or at least one exogenous nucleic acid encoding a phytoene synthase or at least two endogenous nucleic acids encoding a phytoene synthase and/or at least one exogenous nucleic acid encoding a phytoene desaturase or at least two endogenous nucleic acids encoding a phytoene desaturase and/or at least one exogenous nucleic acid encoding a zeta-carotene desaturase or at least two endogenous nucleic acids encoding a zeta-carotene desaturase and/or at least one exogenous nucleic acid encoding a crtISO protein or at least two endogenous nucleic acids encoding a crtISO protein and/or at least one exogenous nucleic acid encoding a FtsZ protein or at least two endogenous nucleic acids encoding a FtsZ protein and/or at least one exogenous nucleic acid encoding a MinD protein or at least two endogenous nucleic acids encoding a MinD protein.

Examples of HMG-CoA reductase genes are:

A nucleic acid encoding an HMG-CoA reductase from Arabidopsis thaliana, Accession NM_{—}106299; (nucleic acid: SEQ ID NO: 19, protein: SEQ ID NO: 20),

and further HMG-CoA reductase genes from other organisms with the following Accession numbers:
P54961, P54870, P54868, P54869, O02734, P22791, P54873, P54871, P23228, P13704, P54872, Q01581, P17425, P54874, P54839, P14891, P34135, O64966, P29057, P48019, P48020, P12683, P43256, Q9XEL8, P34136, O64967, P29058, P48022, Q41437, P12684, Q00583, Q9XHL5, Q41438, Q9YAS4, O76819, O28538, Q9Y7D2, P54960, O51628, P48021, Q03163, P00347, P14773, Q12577, Q59468, P04035, O24594, P09610, Q58116, O26662, Q01237, Q01559, Q12649, O74164, O59469, P51639, Q10283, O08424, P20715, P13703, P13702, Q96UG4, Q8SQZ9, O15888, Q9TUM4, P93514, Q39628, P93081, P93080, Q944T9, Q40148, Q84MM0, Q84LS3, Q9Z9N4, Q9KLM0

Examples of (E)-4-hydroxy-3-methylbut-2-enyl-diphosphate reductase genes are:

A nucleic acid encoding an (E)-4-hydroxy-3-methylbut-2-enyl-diphosphate reductase from Arabidopsis thaliana (IytB/ISPH), ACCESSION AY168881, (nucleic acid: SEQ ID NO: 21, protein: SEQ ID NO:22),

and further (E)-4-hydroxy-3-methylbut-2-enyl-diphosphate reductase genes from other organisms with the following Accession numbers:
T04781, AF270978_{—}1, NP_{—}485028.1, NP_{—}442089.1, NP_{—}681832.1, ZP_{—}0010421.1, ZP_{—}00071594.1, ZP_{—}00114706.1, ISPH_SYNY3, ZP_{—}00114087.1, ZP_{—}00104269.1, AF398145_{—}1, AF398146_{—}1, AAD55762.1, AF514843_{—}1, NP_{—}622970.1, NP_{—}348471.1, NP_{—}562001.1, NP_{—}223698.1, NP_{—}781941.1, ZP_{—}00080042.1, NP_{—}859669.1, NP_{—}214191.1, ZP_{—}00086191.1, ISPH_VIBCH, NP_{—}230334.1, NP_{—}742768.1, NP_{—}302306.1, ISPH_MYCLE, NP_{—}602581.1, ZP_{—}00026966.1, NP_{—}520563.1, NP_{—}253247.1, NP_{—}282047.1, ZP_{—}00038210.1, ZP_{—}00064913.1, CM61555.1, ZP_{—}00125365.1, ISPH_ACICA, EAA24703.1, ZP_{—}00013067.1, ZP_{—}00029164.1, NP_{—}790656.1, NP_{—}217899.1, NP_{—}641592.1, NP_{—}636532.1, NP_{—}719076.1, NP_{—}660497.1, NP_{—}422155.1, NP_{—}715446.1, ZP_{—}00090692.1, NP_{—}759496.1, ISPH_BURPS, ZP_{—}00129657.1, NP_{—}215626.1, NP_{—}335584.1, ZP_{—}00135016.1, NP_{—}789585.1, NP_{—}787770.1, NP_{—}769647.1, ZP 00043336.1, NP_{—}242248.1, ZP_{—}00008555.1, NP_{—}246603.1, ZP_{—}00030951.1, NP_{—}670994.1, NP_{—}404120.1, NP_{—}540376.1, NP_{—}733653.1, NP_{—}697503.1, NP_{—}840730.1, NP_{—}274828.1, NP_{—}796916.1, ZP_{—}00123390.1, NP_{—}824386.1, NP_{—}737689.1, ZP_{—}00021222.1, NP_{—}757521.1, NP_{—}390395.1, ZP_{—}00133322.1, CAD76178.1, NP_{—}600249.1, NP_{—}454660.1, NP_{—}712601.1, NP_{—}385018.1, NP_{—}751989.1

Examples of 1-deoxy-D-xylose-5-phosphate synthase genes are:

A nucleic acid encoding a 1-deoxy-D-xylose-5-phosphate synthase from Lycopersicon esculentum, ACCESSION #AF143812 (nucleic acid: SEQ ID NO:23, protein: SEQ ID NO: 24),

and further 1-deoxy-D-xylose-5-phosphate synthase genes from other organisms with the following Accession numbers:
AF143812_{—}1, DXS_CAPAN, CAD22530.1, AF182286_{—}1, NP_{—}193291.1, T52289, AAC49368.1, MP14353.1, D71420, DXS_ORYSA, AF443590_{—}1, BAB02345.1, CAA09804.2, NP_{—}850620.1, CAD22155.2, AAM65798.1, NP_{—}566686.1, CAD22531.1, MC33513.1, CAC08458.1, MG10432.1, T08140, MP14354.1, AF428463_{—}1, ZP_{—}00010537.1, NP_{—}769291.1, AAK59424.1, NP_{—}107784.1, NP_{—}697464.1, NP_{—}540415.1, NP_{—}196699.1, NP_{—}384986.1, ZP_{—}00096461.1, ZP_{—}00013656.1, NP_{—}353769.1, BM83576.1, ZP_{—}00005919.1, ZP_{—}00006273.1, NP_{—}420871.1, AAM48660.1, DXS_RHOCA, ZP_{—}00045608.1, ZP_{—}00031686.1, NP_{—}841218.1, ZP_{—}00022174.1, ZP_{—}00086851.1, NP_{—}742690.1, NP_{—}520342.1, ZP_{—}00082120.1, NP-790545.1, ZP_{—}00125266.1, CAC17468.1, NP_{—}252733.1, ZP_{—}00092466.1, NP_{—}439591.1, NP_{—}414954.1, NP_{—}752465.1, NP_{—}622918.1, NP_{—}286162.1, NP_{—}836085.1, NP_{—}706308.1, ZP_{—}00081148.1, NP_{—}797065.1, NP_{—}213598.1, NP_{—}245469.1, ZP_{—}00075029.1, NP_{—}455016.1, NP_{—}230536.1, NP_{—}459417.1, NP_{—}274863.1, NP_{—}283402.1, NP_{—}759318.1, NP_{—}406652.1, DXS_SYNLE, DXS_SYNP7, NP_{—}440409.1, ZP_{—}00067331.1, ZP_{—}00122853.1, NP_{—}717142.1, ZP_{—}00104889.1, NP_{—}243645.1, NP_{—}681412.1, DXS_SYNEL, NP_{—}637787.1, DXS_CHLTE, ZP_{—}00129863.1°, NP_{—}661241.1, DXS_XANCP, NP_{—}470738.1, NP_{—}484643.1, ZP_{—}00108360.1, NP_{—}833890.1, NP_{—}846629.1, NP_{—}658213.1, NP_{—}642879.1, ZP_{—}00039479.1, ZP_{—}00060584.1, ZP_{—}00041364.1, ZP_{—}00117779.1, NP_{—}299528.1

Examples of 1-deoxy-D-xylose-5-phosphate reductoisomerase genes are

A nucleic acid encoding a 1-deoxy-D-xylose-5-phosphate reductoisomerase from Arabidopsis thaliana, ACCESSION #AF148852, (nucleic acid: SEQ ID NO: 25, protein: SEQ ID NO: 26),

and further 1-deoxy-D-xylose-5-phosphate reductoisomerase genes from other organisms with the following Accession numbers:
AF148852, AY084775, AY054682, AY050802, AY045634, AY081453, AY091405, AY098952, AJ242588, AB009053, AY202991, NP_{—}201085.1, T52570, AF331705_{—}1, BAB16915.1, AF367205_{—}1, AF250235_{—}1, CAC03581.1, CAD22156.1, AF182287_{—}1, DXR_MENPI, ZP_{—}00071219.1, NP_{—}488391.1, ZP_{—}00111307.1, DXR_SYNLE, AAP56260.1, NP_{—}681831.1, NP_{—}442113.1, ZP_{—}00115071.1, ZP_{—}00105106.1, ZP_{—}00113484.1, NP_{—}833540.1, NP_{—}657789.1, NP_{—}661031.1, DXR_BACHD, NP_{—}833080.1, NP_{—}845693.1, NP_{—}562610.1, NP_{—}623020.1, NP_{—}810915.1, NP_{—}243287.1, ZP_{—}00118743.1, NP_{—}464842.1, NP_{—}470690.1, ZP_{—}00082201.1, NP_{—}781898.1, ZP_{—}00123667.1, NP_{—}348420.1, NP_{—}604221.1, ZP_{—}00053349.1, ZP_{—}00064941.1, NP_{—}246927.1, NP_{—}389537.1, ZP_{—}00102576.1, NP_{—}519531.1, AF124757_{—}19, DXR_ZYMMO, NP_{—}713472.1, NP_{—}459225.1, NP_{—}454827.1, ZP_{—}00045738.1, NP_{—}743754.1, DXR_PSEPK, ZP_{—}00130352.1, NP_{—}702530.1, NP_{—}841744.1, NP_{—}438967.1, AF514841_{—}1, NP_{—}706118.1, ZP_{—}00125845.1, NP_{—}404661.1, NP_{—}285867.1, NP_{—}240064.1, NP_{—}414715.1, ZP_{—}00094058.1, NP_{—}791365.1, ZP_{—}00012448.1, ZP_{—}00015132.1, ZP_{—}00091545.1, NP_{—}629822.1, NP_{—}771495.1, NP_{—}798691.1, NP_{—}231885.1, NP_{—}252340.1, ZP_{—}00022353.1, NP_{—}355549.1, NP_{—}420724.1, ZP_{—}00085169.1, EAA17616.1, NP_{—}273242.1, NP_{—}219574.1, NP_{—}387094.1, NP_{—}296721.1, ZP_{—}00004209.1, NP_{—}823739.1, NP_{—}282934.1, BM77848.1, NP_{—}660577.1, NP_{—}760741.1, NP_{—}641750.1, NP_{—}636741.1, NP_{—}829309.1, NP_{—}298338.1, NP_{—}444964.1, NP_{—}717246.1, NP_{—}224545.1, ZP_{—}00038451.1, DXR_KITGR, NP_{—}778563.1.

Examples of isopentenyl-diphosphate Δ-isomerase genes are

A nucleic acid encoding an isopentenyl-diphosphate Δ-isomerase from Adonis palaestina clone ApIPI28, (ipiAal), ACCESSION #AF188060, published by Cunningham, F. X. Jr. and Gantt, E.: Identification of multi-gene families encoding isopentenyl diphosphate isomerase in plants by heterologous complementation in Escherichia coli, Plant Cell Physiol. 41 (1), 119-123 (2000) (nucleic acid: SEQ ID NO: 27, protein: SEQ ID NO: 28),

and further isopentenyl-diphosphate Δ-isomerase genes from other organisms with the following Accession numbers:
Q38929, O48964, Q39472, Q13907, O35586, P58044, O42641, O35760, Q10132, P15496, Q9YB30, Q8YNH4, Q42553, O27997, P50740, O51627, O48965, Q8KFR5, Q39471, Q39664, Q9RVE2, Q01335, Q9HHE4, Q9BXS1, Q9 KWF6, Q9CIF5, Q88WB6, Q92BX2, Q8Y7A5, Q8TT35 Q9KK75, Q8NN99, Q8XD58, Q8FE75, Q46822, Q9HP40, P72002, P26173, Q9Z5D3, Q8Z3×9, Q8ZM82, Q9X7Q6, O13504, Q9HFW8, Q8NJL9, Q9UUQ1, Q9NHO2, Q9M6K9, Q9M6K5, Q9FXR6, O81691, Q9S7C4, Q8S3L8, Q9M592, Q9M6K3, Q9M6K7, Q9FV48, Q9LLB6, Q9AVJ1, Q9AVG8, Q9M6K6, Q9AVJ5, Q9M6K2, Q9AYS5, Q9M6K8, Q9AVG7, Q8S3L7, Q8W250, Q94IE1, Q9AV18, Q9AYS6, Q9SAY0, Q9M6K4, Q8GVZ0, Q84RZ8, Q8KZ12, Q8KZ66, Q8FND7, Q88QC9, Q8BFZ6, BAC26382, CAD94476.

Examples of geranyl-diphosphate synthase genes are:

A nucleic acid encoding a geranyl-diphosphate synthase from Arabidopsis thaliana, ACCESSION #Y17376, Bouvier, F., Suire, C., d'Harlingue, A., Backhaus, R. A. and Camara, B.; Molecular cloning of geranyl diphosphate synthase and compartmentation of monoterpene synthesis in plant cells, Plant J. 24 (2), 241-252 (2000) (nucleic acid: SEQ ID NO: 29, protein: SEQ ID NO: 30),

and further geranyl-diphosphate synthase genes from other organisms with the following Accession numbers:

### Q9FT89, Q8LKJ2, Q9FSW8, Q8LKJ3, Q9SBR3, Q9SBR4, Q9FET8, Q8LKJ1, Q84LG1, Q9JK86

Examples of farnesyl-diphosphate synthase genes are:

A nucleic acid encoding a farnesyl-diphosphate synthase from Arabidopsis thaliana (FPS1), ACCESSION #U80605, published by Cunillera, N., Arro, M., Delourme, D., Karst, F., Boronat, A. and Ferrer, A.: Arabidopsis thaliana contains two differentially expressed farnesyl-diphosphate synthase genes, J. Biol. Chem. 271 (13), 7774-7780 (1996), (nucleic acid: SEQ ID NO: 31, protein: SEQ ID NO: 32), and further farnesyl-diphosphate synthase genes from other organisms with the following Accession numbers:

P53799, P37268, Q02769, Q09152, P49351, O24241, Q43315, P49352, O24242, P49350, P08836, P14324, P49349, P08524, O66952, Q08291, P54383, Q45220, P57537, Q8K9A0, P22939, P45204, O66126, P55539, Q9SWH9, Q9AVI7, Q9FRX2, Q9AYS7, Q941E8, Q9FXR9, Q9ZWF6, Q9FXR8, Q9AR37, O50009, Q941E9, Q8RVK7, Q8RVQ7, O04882, Q93RA8, Q93RB0, Q93RB4, Q93RB5, Q93RB3, Q93RB1, Q93RB2, Q920E5.

Examples of geranylgeranyl-diphosphate synthase genes are:

A nucleic acid encoding a geranylgeranyl-diphosphate synthase from Sinaps alba, ACCESSION #X98795, published by Bonk, M., Hoffmann, B., Von Lintig, J., Schledz, M., Al-Babili, S., Hobeika, E., Kleinig, H. and Beyer, P.: Chloroplast import of four carotenoid biosynthetic enzymes in vitro reveals differential fates prior to membrane binding and oligomeric assembly, Eur. J. Biochem. 247 (3), 942-950 (1997), (nucleic acid: SEQ ID NO: 33, protein: SEQ ID NO: 34),

and further geranylgeranyl-diphosphate synthase genes from other organisms with the following Accession numbers:
P22873, P34802, P56966, P80042, Q42698, Q92236, O95749, Q9WTN0, Q50727, P24322, P39464, Q9FXR3, Q9AYN2, Q9FXR2, Q9AVG6, Q9FRW4, Q9SXZ5, Q9AVJ7, Q9AYN1, Q9AVJ4, Q9FXR7, Q8LSC5, Q9AVJ6, Q8LSC4, Q9AVJ3, Q9SSU0, Q9SXZ6, Q9SST9, Q9AVJ0, Q9AV19, Q9FRW3, Q9FXR5, Q941F0, Q9FRX1, Q9K567, Q93RA9, Q93QX8, CAD95619, EM31459

Examples of phytoene synthase genes are:

A nucleic acid encoding a phytoene synthase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 35, protein: SEQ ID NO: 36),

and further phytoene synthase genes from other organisms with the following Accession numbers:
CAB39693, BAC69364, AAF10440, CAA45350, BAA20384, AAM72615, BAC09112, CAA48922, P_{—}001091, CAB84588, MF41518, CAA48155, AAD38051, MF33237, AAG10427, AAA34187, BAB73532, CAC19567, AAM62787, CAA55391, MB65697, AAM45379, CAC27383, AAA32836, AAK07735, BM84763, P_{—}000205, MB60314, P_{—}001163, P_{—}000718, MB71428, AAA34153, AAK07734, CAA42969, CAD76176, CM68575, P_{—}000130, P_{—}001142, CM47625, CM85775, BAC14416, CAA79957, BAC76563, P_{—}000242, P_{—}000551, ML02001, AAK15621, CAB94795, AAA91951, P_{—}000448

Examples of phytoene desaturase genes are:

A nucleic acid encoding a phytoene desaturase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 37, protein: SEQ ID NO: 38), and further phytoene desaturase genes from other organisms with the following Accession numbers:

AAL15300, A39597, CM42573, AAK51545, BAB08179, CM48195, BAB82461, AAK92625, CAA55392, MG10426, MD02489, M024235, MC12846, AAA99519, AAL38046, CM60479, CM75094, ZP_{—}001041, ZP_{—}001163, CAA39004, CM44452, ZP_{—}001142, ZP_{—}000718, BAB82462, AAM45380, CAB56040, ZP_{—}001091, BAC09113, AAP79175, AAL80005, AAM72642, AAM72043, ZP_{—}000745, ZP_{—}001141, BAC07889, CAD55814, ZP_{—}001041, CAD27442, CAE00192, ZP_{—}001163, ZP_{—}000197, BM18400, AAG10425, ZP_{—}001119, AAF13698, 2121278A, AAB35386, AAD02462, BAB68552, CAC85667, MK51557, CM12062, MG51402, MM63349, AAF85796, BAB74081, AAA91161, CAB56041, AAC48983, AAG14399, CAB65434, BAB73487, ZP_{—}001117, ZP_{—}000448, CAB39695, CAD76175, BAC69363, BM17934, ZP_{—}000171, MF65586, ZP_{—}000748, BAC07074, ZP_{—}001133, CAA64853, BAB74484, ZP_{—}001156, MF23289, AAG28703, MP09348, AAM71569, BAB69140, ZP_{—}000130, AAF41516, MG18866, CAD95940, NP_{—}656310, AAG10645, ZP_{—}000276, ZP_{—}000192, ZP_{—}000186, AAM94364, EAA31371, ZP_{—}000612, BAC75676, AAF65582

Examples of zeta-carotene desaturase genes are:

A nucleic acid encoding a zeta-carotene desaturase from Narcissus pseudonarcissus, ACCESSION #AJ224683, published by Al-Babili, S., Oelschlegel, J. and Beyer, P.: A cDNA encoding for beta carotene desaturase (Accession No.AJ224683) from Narcissus pseudonarcissus L. (PGR98-103), Plant Physiol. 117, 719-719 (1998), (nucleic acid: SEQ ID NO: 39, protein: SEQ ID NO: 40),

and further zeta-carotene desaturase genes from other organisms with the following Accession numbers:
Q9R6X4, Q38893, Q9SMJ3, Q9SE20, Q9ZTP4, O49901, P74306, Q9FV46, Q9RCT2, ZDS_NARPS, BAB68552.1, CAC85667.1, AF372617_{—}1, ZDS_TARER, CAD55814.1, CAD27442.1, 2121278A, ZDS_CAPAN, ZDS_LYCES, NP_{—}187138.1, AAM63349.1, ZDS_ARATH, AAA91161.1, ZDS_MAIZE, MG14399.1, NP_{—}441720.1, NP_{—}486422.1, ZP_{—}00111920.1, CAB56041.1, ZP_{—}00074512.1, ZP_{—}00116357.1, NP_{—}681127.1, ZP_{—}00114185.1, ZP_{—}00104126.1, CAB65434.1, NP_{—}662300.1

Examples of crtISO genes are:

A nucleic acid encoding a crtISO from Lycopersicon esculentum; ACCESSION #AF416727, published by Isaacson, T., Ronen, G., Zamir, D. and Hirschberg, J.: Cloning of tangerine from tomato reveals a carotenoid isomerase essential for the production of beta-carotene and xanthophylls in plants; Plant Cell 14 (2), 333-342 (2002), (nucleic acid: SEQ ID NO: 41, protein: SEQ ID NO:42),

and further crtISO genes from other organisms with the followng Accession numbers:

### AAM53952

Examples of FtsZ genes are:

A nucleic acid encoding a FtsZ from Tagetes erecta, ACCESSION #AF251346, published by Moehs, C. P., Tian, L., Osteryoung, K. W. and Dellapenna, D.: Analysis of carotenoid biosynthetic gene expression during marigold petal development Plant Mol. Biol. 45 (3), 281-293 (2001), (nucleic acid: SEQ ID NO: 43, protein: SEQ ID NO: 44),

and further FtsZ genes from other organisms with the following Accession numbers:
CAB89286.1, AF205858_{—}1, NP_{—}200339.1, CAB89287.1, CAB41987.1, AAA82068.1, T06774, AF383876_{—}1, BAC57986.1, CAD22047.1, BAB91150.1, ZP_{—}00072546.1, NP_{—}440816.1, T51092, NP_{—}683172.1, BAA85116.1, NP_{—}487898.1, JC4289, BAA82871.1, NP_{—}781763.1, BAC57987.1, ZP_{—}00111461.1, T51088, NP_{—}190843.1, ZP_{—}00060035.1, NP_{—}846285.1, ML07180.1, NP_{—}243424.1, NP_{—}833626.1, AAN04561.1, AAN04557.1, CAD22048.1, T51089, NP_{—}692394.1, NP_{—}623237.1, NP_{—}565839.1, T51090, CAA07676.1, NP_{—}113397.1, T51087, CAC44257.1, E84778, ZP_{—}00105267.1, BAA82091.1, ZP_{—}00112790.1, BAA96782.1, NP_{—}348319.1, NP_{—}471472.1, ZP_{—}00115870.1, NP_{—}465556.1, NP_{—}389412.1, BAA82090.1, NP_{—}562681.1, AAM22891.1, NP_{—}371710.1, NP_{—}764416.1, CAB95028.1, FTSZ_STRGR, AF120117_{—}1, NP_{—}827300.1, JE0282, NP_{—}626341.1, MC45639.1, NP_{—}785689.1, NP_{—}336679.1, NP_{—}738660.1, ZP_{—}00057764.1, MC32265.1, NP_{—}814733.1, FTSZ_MYCKA, NP_{—}216666.1, CM75616.1, NP_{—}301700.1, NP_{—}601357.1, ZP_{—}00046269.1, CM70158.1, ZP_{—}00037834.1, NP_{—}268026.1, FTSZ_ENTHR, NP_{—}787643.1, NP_{—}346105.1, AAC32264.1, JC5548, MC95440.1, NP_{—}710793.1, NP_{—}687509.1, NP_{—}269594.1, AAC32266.1, NP_{—}720988.1, NP_{—}657875.1, ZP_{—}00094865.1, ZP_{—}00080499.1, ZP_{—}00043589.1, JC7087, NP_{—}660559.1, AAC46069.1, AF179611_{—}14, AAC44223.1, NP_{—}404201.1.

Examples of MinD genes are:

A nucleic acid encoding a MinD from Tagetes erecta, ACCESSION #AF251019, published by Moehs, C. P., Tian, L., Osteryoung, K. W. and Dellapenna, D.: Analysis of carotenoid biosynthetic gene expression during marigold petal development; Plant Mol. Biol. 45 (3), 281-293 (2001), (nucleic acid: SEQ ID NO: 45, protein: SEQ ID NO: 46),

and further MinD genes with the following Accession numbers:
NP_{—}197790.1, BAA90628.1, NP_{—}038435.1, NP_{—}045875.1, MN33031.1, NP_{—}050910.1, CAB53105.1, NP_{—}050687.1, NP_{—}682807.1, NP_{—}487496.1, ZP_{—}00111708.1, ZP_{—}00071109.1, NP_{—}442592.1, NP_{—}603083.1, NP_{—}782631.1, ZP_{—}00097367.1, ZP_{—}00104319.1, NP_{—}294476.1, NP_{—}622555.1, NP_{—}563054.1, NP_{—}347881.1, ZP_{—}00113908.1, NP_{—}834154.1, NP_{—}658480.1, ZP_{—}00059858.1, NP_{—}470915.1, NP_{—}243893.1, NP_{—}465069.1, ZP_{—}00116155.1, NP_{—}390677.1, NP_{—}692970.1, NP_{—}298610.1, NP_{—}207129.1, ZP_{—}00038874.1, NP_{—}778791.1, NP_{—}223033.1, NP_{—}641561.1, NP_{—}636499.1, ZP_{—}00088714.1, NP_{—}213595.1, NP_{—}743889.1, NP_{—}231594.1, ZP_{—}00085067.1, NP_{—}797252.1, ZP_{—}00136593.1, NP_{—}251934.1, NP_{—}405629.1, NP_{—}759144.1, ZP_{—}00102939.1, NP_{—}793645.1, NP_{—}699517.1, NP_{—}460771.1, NP_{—}860754.1, NP_{—}456322.1, NP_{—}718163.1, NP_{—}229666.1, NP_{—}357356.1, NP_{—}541904.1, NP_{—}287414.1, NP_{—}660660.1, ZP_{—}00128273.1, NP_{—}103411.1, NP_{—}785789.1, NP_{—}715361.1, AF149810_{—}1, NP_{—}841854.1, NP_{—}437893.1, ZP_{—}00022726.1, EAA24844.1, ZP_{—}00029547.1, NP_{—}521484.1, NP_{—}240148.1, NP_{—}770852.1, AF345908_{—}2, NP_{—}777923.1, ZP_{—}00048879.1, NP_{—}579340.1, NP_{—}143455.1, NP_{—}126254.1, NP_{—}142573.1, NP_{—}613505.1, NP_{—}127112.1, NP_{—}712786.1, NP_{—}578214.1, NP_{—}069530.1, NP_{—}247526.1, AAA85593.1, NP_{—}212403.1, NP_{—}782258.1, ZP_{—}00058694.1, NP_{—}247137.1, NP_{—}219149.1, NP_{—}276946.1, NP_{—}614522.1, ZP_{—}00019288.1, CAD78330.1

The HMG-CoA reductase genes preferably used in the preferred embodiment described above are nucleic acids which encode proteins comprising the amino acid sequence SEQ ID NO: 20 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 20, and having the enzymatic property of an HMG-CoA reductase.

Further examples of HMG-CoA reductases and HMG-CoA reductase genes can easily be found for example from various organisms whose genomic sequence is known, as described above, by homologous comparisons of the amino acid sequences or of the corresponding back-translated nucleic acid sequences from databases with SeQ ID NO: 20.

Further examples of HMG-CoA reductases and HMG-CoA reductase genes can moreover easily be found for example starting from the sequence SEQ ID NO: 19 from various organisms whose genomic sequence is unknown, as described above, by hybridization and PCR techniques in a manner known per se.

In a further particularly preferred embodiment, the HMG-CoA reductase activity is raised by introducing nucleic acids into organisms which encode proteins comprising the amino acid sequence of the HMG-CoA reductase of the sequence SEQ ID NO: 20.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 19 is introduced into the organism.

The (E)-4-hydroxy-3-methylbut-2-enyl-diphosphate reductase genes preferably used in the preferred embodiment described above are nucleic acids which encode proteins comprising the amino acid sequence SEQ ID NO: 22 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 22, and having the enzymatic property of an (E)-4-hydroxy-3-methylbut-2-enyl-diphosphate reductase.

Further examples of (E)-4-hydroxy-3-methylbut-2-enyl-diphosphate reductases and (E)-4-hydroxy-3-methylbut-2-enyl-diphosphate reductase genes can easily be found for example from various organisms whose genomic sequence is known, as described above, by homologous comparisons of the amino acid sequences or of the corresponding back-translated nucleic acid sequences from databases with SeQ ID NO: 22.

Further examples of (E)-4-hydroxy-3-methylbut-2-enyl-diphosphate reductases and (E)-4-hydroxy-3-methylbut-2-enyl-diphosphate reductase genes can moreover easily be found for example starting from the sequence SEQ ID NO: 21 from various organisms whose genomic sequence is unknown, as described above, by hybridization and PCR techniques in a manner known per se.

In a further particularly preferred embodiment, the (E)-4-hydroxy-3-methylbut-2-enyl-diphosphate reductase activity is raised by introducing nucleic acids into organisms which encode proteins comprising the amino acid sequence of the (E)-4-hydroxy-3-methylbut-2-enyl-diphosphate reductase of the sequence SEQ ID NO: 22.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 21 is introduced into the organism.

The 1-deoxy-D-xylose-5-phosphate synthase genes preferably used in the preferred embodiment described above are nucleic acids which encode proteins comprising the amino acid sequence SEQ ID NO: 24 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 24, and having the enzymatic property of a 1-deoxy-D-xylose-5-phosphate synthase.

Further examples of 1-deoxy-D-xylose-5-phosphate synthases and 1-deoxy-D-xylose-5-phosphate synthase genes can easily be found for example from various organisms whose genomic sequence is known, as described above, by homologous comparisons of the amino acid sequences or of the corresponding back-translated nucleic acid sequences from databases with SeQ ID NO: 24.

Further examples of 1-deoxy-D-xylose-5-phosphate synthases and 1-deoxy-D-xylose-5-phosphate synthase genes can moreover easily be found for example starting from the sequence SEQ ID NO: 23 from various organisms whose genomic sequence is unknown, as described above, by hybridization and PCR techniques in a manner known per se.

In a further particularly preferred embodiment, the 1-deoxy-D-xylose-5-phosphate synthase activity is raised by introducing nucleic acids into organisms which encode proteins comprising the amino acid sequence of the 1-deoxy-D-xylose-5-phosphate synthase of the sequence SEQ ID NO: 24.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 23 is introduced into the organism.

The 1-deoxy-D-xylose-5-phosphate reductoisomerase genes preferably used in the preferred embodiment described above are nucleic acids which encode proteins comprising the amino acid sequence SEQ ID NO: 26 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 26, and having the enzymatic property of a 1-deoxy-D-xylose-5-phosphate reductoisomerase.

Further examples of 1-deoxy-D-xylose-5-phosphate reductoisomerases and 1-deoxy-D-xylose-5-phosphate reductoisomerase genes can easily be found for example from various organisms whose genomic sequence is known, as described above, by homologous comparisons of the amino acid sequences or of the corresponding back-translated nucleic acid sequences from databases with SeQ ID NO: 26.

Further examples of 1-deoxy-D-xylose-5-phosphate reductoisomerases and 1-deoxy-D-xylose-5-phosphate reductoisomerase genes can moreover easily be found for example starting from the sequence SEQ ID NO: 25 from various organisms whose genomic sequence is unknown, as described above, by hybridization and PCR techniques in a manner known per se.

In a further particularly preferred embodiment, the 1-deoxy-D-xylose-5-phosphate reductoisomerase activity is raised by introducing nucleic acids into organisms which encode proteins comprising the amino acid sequence of the 1-deoxy-D-xylose-5-phosphate reductoisomerase of the sequence SEQ ID NO: 26.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 25 is introduced into the organism.

The isopentenyl D-isomerase genes preferably used in the preferred embodiment described above are nucleic acids which encode proteins comprising the amino acid sequence SEQ ID NO: 28 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 28, and having the enzymatic property of an isopentenyl D-isomerase.

Further examples of isopentenyl D-isomerases and isopentenyl D-isomerase genes can easily be found for example from various organisms whose genomic sequence is known, as described above, by homologous comparisons of the amino acid sequences or of the corresponding back-translated nucleic acid sequences from databases with SeQ ID NO: 28.

Further examples of isopentenyl D-isomerases and isopentenyl D-isomerase genes can moreover easily be found for example starting from the sequence SEQ ID NO: 27 from various organisms whose genomic sequence is unknown, as described above, by hybridization and PCR techniques in a manner known per se.

In a further particularly preferred embodiment, the isopentenyl D-isomerase activity is raised by introducing nucleic acids into organisms which encode proteins comprising the amino acid sequence of the isopentenyl D-isomerase of the sequence SEQ ID NO: 28.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms. In a particularly preferred embodiment, a nucleic acid comprising the sequence SEQ ID NO: 27 is introduced into the organism.

The geranyl-diphosphate synthase genes preferably used in the preferred embodiment described above are nucleic acids which encode proteins comprising the amino acid sequence SEQ ID NO: 30 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 30, and having the enzymatic property of a geranyl-diphosphate synthase.

Further examples of geranyl-diphosphate synthases and geranyl-diphosphate synthase genes can easily be found for example from various organisms whose genomic sequence is known, as described above, by homologous comparisons of the amino acid sequences or of the corresponding back-translated nucleic acid sequences from databases with SeQ ID NO: 30.

Further examples of geranyl-diphosphate synthases and geranyl-diphosphate synthase genes can moreover easily be found for example starting from the sequence SEQ ID NO: 29 from various organisms whose genomic sequence is unknown, as described above, by hybridization and PCR techniques in a manner known per se.

In a further particularly preferred embodiment, the geranyl-diphosphate synthase activity is raised by introducing nucleic acids into organisms which encode proteins comprising the amino acid sequence of the geranyl-diphosphate synthase of the sequence SEQ ID NO: 30.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 29 is introduced into the organism.

The farnesyl-diphosphate synthase genes preferably used in the preferred embodiment described above are nucleic acids which encode proteins comprising the amino acid sequence SEQ ID NO: 32 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 32, and having the enzymatic property of a farnesyl-diphosphate synthase.

Further examples of farnesyl-diphosphate synthases and farnesyl-diphosphate synthase genes can easily be found for example from various organisms whose genomic sequence is known, as described above, by homologous comparisons of the amino acid sequences or of the corresponding back-translated nucleic acid sequences from databases with SeQ ID NO: 32.

Further examples of farnesyl-diphosphate synthases and farnesyl-diphosphate synthase genes can moreover easily be found for example starting from the sequence SEQ ID NO: 31 from various organisms whose genomic sequence is unknown, as described above, by hybridization and PCR techniques in a manner known per se.

In a further particularly preferred embodiment, the farnesyl-diphosphate synthase activity is raised by introducing nucleic acids into organisms which encode proteins comprising the amino acid sequence of the farnesyl-diphosphate synthase of the sequence SEQ ID NO: 32.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 31 is introduced into the organism.

The geranylgeranyl-diphosphate synthase genes preferably used in the preferred embodiment described above are nucleic acids which encode proteins comprising the amino acid sequence SEQ ID NO: 34 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 34, and having the enzymatic property of a geranylgeranyl-diphosphate synthase.

Further examples of geranylgeranyl-diphosphate synthases and geranylgeranyl-diphosphate synthase genes can easily be found for example from various organisms whose genomic sequence is known, as described above, by homologous comparisons of the amino acid sequences or of the corresponding back-translated nucleic acid sequences from databases with SeQ ID NO: 22.

Further examples of geranylgeranyl-diphosphate synthases and geranylgeranyl-diphosphate synthase genes can moreover easily be found for example starting from the sequence SEQ ID NO: 33 from various organisms whose genomic sequence is unknown, as described above, by hybridization and PCR techniques in a manner known per se.

In a further particularly preferred embodiment, the geranylgeranyl-diphosphate synthase activity is raised by introducing nucleic acids into organisms which encode proteins comprising the amino acid sequence of the geranylgeranyl-diphosphate synthase of the sequence SEQ ID NO: 34.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 33 is introduced into the organism.

The phytoene synthase genes preferably used in the preferred embodiment described above are nucleic acids which encode proteins comprising the amino acid sequence SEQ ID NO: 36 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 36, and having the enzymatic property of a phytoene synthase.

Further examples of phytoene synthases and phytoene synthase genes can easily be found for example from various organisms whose genomic sequence is known, as described above, by homologous comparisons of the amino acid sequences or of the corresponding back-translated nucleic acid sequences from databases with SeQ ID NO: 36.

Further examples of phytoene synthases and phytoene synthase genes can moreover easily be found for example starting from the sequence. SEQ ID NO: 35 from various organisms whose genomic sequence is unknown, as described above, by hybridization and PCR techniques in a manner known per se.

In a further particularly preferred embodiment, the phytoene synthase activity is raised by introducing nucleic acids into organisms which encode proteins comprising the amino acid sequence of the phytoene synthase of the sequence SEQ ID NO: 36.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 35 is introduced into the organism.

The phytoene desaturase genes preferably used in the preferred embodiment described above are nucleic acids which encode proteins comprising the amino acid sequence SEQ ID NO: 38 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 38, and having the enzymatic property of a phytoene desaturase.

Further examples of phytoene desaturases and phytoene desaturase genes can easily be found for example from various organisms whose genomic sequence is known, as described above, by homologous comparisons of the amino acid sequences or of the corresponding back-translated nucleic acid sequences from databases with SeQ ID NO: 38.

Further examples of phytoene desaturases and phytoene desaturase genes can moreover easily be found for example starting from the sequence SEQ ID NO: 37 from various organisms whose genomic sequence is unknown, as described above, by hybridization and PCR techniques in a manner known per se.

In a further particularly preferred embodiment, the phytoene desaturase activity is raised by introducing nucleic acids into organisms which encode proteins comprising the amino acid sequence of the phytoene desaturase of the sequence SEQ ID NO: 38.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 37 is introduced into the organism.

The zeta-carotene desaturase genes preferably used in the preferred embodiment described above are nucleic acids which encode proteins comprising the amino acid sequence SEQ ID NO: 40 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 40, and having the enzymatic property of a zeta-carotene desaturase.

Further examples of zeta-carotene desaturases and zeta-carotene desaturase genes can easily be found for example from various organisms whose genomic sequence is known, as described above, by homologous comparisons of the amino acid sequences or of the corresponding back-translated nucleic acid sequences from databases with SeQ ID NO: 40.

Further examples of zeta-carotene desaturases and zeta-carotene desaturase genes can moreover easily be found for example starting from the sequence SEQ ID NO: 39 from various organisms whose genomic sequence is unknown, as described above, by hybridization and PCR techniques in a manner known per se.

In a further particularly preferred embodiment, the zeta-carotene desaturase activity is raised by introducing nucleic acids into organisms which encode proteins comprising the amino acid sequence of the zeta-carotene desaturase of the sequence SEQ ID NO: 40.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms. In a particularly preferred embodiment, a nucleic acid comprising the sequence SEQ ID NO: 39 is introduced into the organism.

The CrtISO genes preferably used in the preferred embodiment described above are nucleic acids which encode proteins comprising the amino acid sequence SEQ ID NO: 42 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 42, and having the enzymatic property of a CrtISO.

Further examples of CrtISOs and CrtISO genes can easily be found for example from various organisms whose genomic sequence is known, as described above, by homologous comparisons of the amino acid sequences or of the corresponding back-translated nucleic acid sequences from databases with SeQ ID NO: 42.

Further examples of CrtISOs and CrtISO genes can moreover easily be found for example starting from the sequence SEQ ID NO: 41 from various organisms whose genomic sequence is unknown, as described above, by hybridization and PCR techniques in a manner known per se.

In a further particularly preferred embodiment, the CrtISO reductase activity is raised by introducing nucleic acids into organisms which encode proteins comprising the amino acid sequence of the CrtISO of the sequence SEQ ID NO: 42.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 41 is introduced into the organism.

The FtsZ genes preferably used in the preferred embodiment described above are nucleic acids which encode proteins comprising the amino acid sequence SEQ ID NO: 44 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 44, and having the enzymatic property of an FtsZ.

Further examples of FtsZs and FtsZ genes can easily be found for example from various organisms whose genomic sequence is known, as described above, by homologous comparisons of the amino acid sequences or of the corresponding back-translated nucleic acid sequences from databases with SeQ ID NO: 44.

Further examples of FtsZs and FtsZ genes can moreover easily be found for example starting from the sequence SEQ ID NO: 43 from various organisms whose genomic sequence is unknown, as described above, by hybridization and PCR techniques in a manner known per se.

In a further particularly preferred embodiment, the FtsZ activity is raised by introducing nucleic acids into organisms which encode proteins comprising the amino acid sequence of the FtsZ of the sequence SEQ ID NO: 44.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 43 is introduced into the organism.

The MinD genes preferably used in the preferred embodiment described above are nucleic acids which encode proteins comprising the amino acid sequence SEQ ID NO: 46 or a sequence derived from this sequence by substitution, insertion or deletion of amino acids and having an identity of at least 30%, preferably at least 50%, more preferably at least 70%, even more preferably at least 90%, most preferably at least 95% at the amino acid level with the sequence SEQ ID NO: 46, and having the enzymatic property of an MinD.

Further examples of MinDs and MinD genes can easily be found for example from various organisms whose genomic sequence is known, as described above, by homologous comparisons of the amino acid sequences or of the corresponding back-translated nucleic acid sequences from databases with SeQ ID NO: 46.

Further examples of MinDs and MinD genes can moreover easily be found for example starting from the sequence SEQ ID NO: 45 from various organisms whose genomic sequence is unknown, as described above, by hybridization and PCR techniques in a manner known per se.

In a further particularly preferred embodiment, the MinD activity is raised by introducing nucleic acids into organisms which encode proteins comprising the amino acid sequence of the MinD of the sequence SEQ ID NO: 46.

Suitable nucleic acid sequences can be obtained for example by back-translation of the polypeptide sequence in accordance with the genetic code.

The codons preferably used for this purpose are those frequently used according to the organism-specific codon usage. The codon usage can easily be ascertained by means of computer analyses of other, known genes of the relevant organisms.

In a particularly preferred embodiment, a nucleic acid comprising the sequence SEQ ID NO: 45 is introduced into the organism.

All the aforementioned HMG-CoA reductase genes, (E)-4-hydroxy-3-methylbut-2-enyl-diphosphate reductase genes, 1-deoxy-D-xylose-5-phosphate synthase genes, 1-deoxy-D-xylose-5-phosphate reductoisomerase genes, isopentenyl-diphosphate Δ-isomerase genes, geranyl-diphosphate synthase genes, farnesyl-diphosphate synthase genes, geranylgeranyl-diphosphate synthase genes, phytoene synthase genes, phytoene desaturase genes, zeta-carotene desaturase genes, crtISO genes, FtsZ genes or MinD genes can moreover be prepared in a manner known per se by chemical synthesis from the nucleotide units such as, for example, by fragment condensation of individual overlapping, complementary nucleic acid units of the double helix. Chemical synthesis of oligonucleotides can take place for example in a known manner by the phosphoamidite method (Voet, Voet, 2nd edition, Wiley Press New York, pages 896-897). The addition of synthetic oligonucleotides and filling in of gaps using the Klenow fragment of DNA polymerase and ligation reactions, and general cloning methods are described in Sambrook et al. (1989), Molecular cloning: A laboratory manual, Cold Spring Harbor Laboratory Press.

The nucleic acids encoding a ketolase selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14, and
   nucleic acids encoding a β-hydroxylase, nucleic acids encoding a β-cyclase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate Δ-isomerase, nucleic acids encoding a geranyl-diphosphate synthase, nucleic acids encoding a farnesyl-diphosphate synthase, nucleic acids encoding a geranylgeranyl-diphosphate synthase, nucleic acids encoding a phytoene synthase, nucleic acids encoding a phytoene desaturase, nucleic acids encoding a zeta-carotene desaturase, nucleic acids encoding a crtISO protein, nucleic acids encoding a FtsZ protein and/or nucleic acids encoding a MinD protein are also called “effect genes” hereinafter.

The genetically modified, non-human organisms can be produced as described below for example by introducing individual nucleic acid constructs (expression cassettes) comprising an effect gene, or by introducing multiple constructs which comprise up to two or three or more of the effect genes.

Organisms mean according to the invention preferably organisms which as wild-type or initial organims are able, naturally or through genetic complementation and/or rerouting of metabolic pathways, to produce carotenoids, especially β-carotene and/or zeaxanthin and/or neoxanthin and/or violaxanthin and/or lutein.

Further preferred organisms already have as wild-type or initial organisms a hydroxylase activity and are thus able as wild-type or initial organisms to produce zeaxanthin.

Preferred organisms are plants or microorganisms such as, for example, bacteria, yeasts, algae or fungi.

Bacteria which can be used are both bacteria which are able, owing to the introduction of genes of carotenoid biosynthesis from a carotenoid-producing organism, to synthesize xanthophylls, such as, for example, bacteria of the genus Escherichia which comprise, for example, crt genes from Erwinia, and bacteria intrinsically able to synthesize xanthophylls, such as, for example, bacteria of the genus Erwinia, Agrobacterium, Flavobacterium, Alcaligenes, Paracoccus, Nostoc or cyanobacteria of the genus Synechocystis.

Preferred bacteria are Escherichia coli, Erwinia herbicola, Erwinia uredovora, Agrobacterium aurantiacum, Alcaligenes sp. PC-1, Flavobacterium sp. strain R1534, the cyanobacterium Synechocystis sp. PCC6803, Paracoccus marcusli or Paracoccus carotinifaciens.

Preferred yeasts are Candida, Saccharomyces, Hansenula, Pichia or Phaffia. Particularly preferred yeasts are Xanthophyllomyces dendrorhous or Phaffia rhodozyma.

Preferred fungi are Aspergillus, Trichoderma, Ashbya, Neurospora, Blakeslea, especially Blakeslea trispora, Phycomyces, Fusarium or further fungi described in Indian Chem. Engr. Section B. Vol. 37, No. 1, 2 (1995) on page 15, Table 6.

Preferred algae are green algae such as, for example, algae of the genus Haematococcus, Phaedactylum tricomatum, Volvox or Dunaliella. Particularly preferred algae are Haematococcus puvialis or Dunaliella bardawil.

Further useful microorganisms and their production for carrying out the process of the invention are disclosed for example in DE-A-199 16 140, which is incorporated herein by reference.

In a particularly preferred embodiment, plants are used as non-human organisms.

In a particularly preferred embodiment of the process of the invention, genetically modified plants which have the highest expression rate of a ketolase of the invention in flowers are used.

This is preferably achieved by expression of the ketolase gene of the invention taking place under the control of flower-specific promoter. For this purpose, for example, the nucleic acids described above are introduced, as described in detail below, in a nucleic acid construct functionally linked to a flower-specific promoter into the plant.

In a further preferred embodiment of the process using plants, the genetically modified plants additionally have a reduced ε-cyclase activity compared with the wild type.

ε-Cyclase activity means the enzymic activity of an ε-cyclase.

An ε-cyclase means a protein which has the enzymatic activity of converting a terminal linear residue of lycopene into an ε-ionone ring.

An ε-cyclase therefore means in particular a protein having the enzymatic activity of converting lycopene into δ-carotene.

Accordingly, the ε-cyclase activity is the amount of lycopene converted or amount of δ-carotene formed in a particular time by the ε-cyclase protein.

Thus, when the ε-cyclase activity is reduced compared with the wild type, the amount of lycopene converted or the amount of δ-carotene formed in a particular time by the ε-cyclase protein is reduced by comparison with the wild type.

A reduced ε-cyclase activity preferably means the partial or substantially complete suppression or blocking, based on various cell-biology mechanisms, of the functionality of an ε-cyclase in a plant cell, plant or a part, tissue, organ, cells or seeds derived therefrom.

The reduction in the ε-cyclase activity in plants compared with the wild type can take place for example by reducing the amount of ε-cyclase protein or the amount of ε-cyclase mRNA in the plant. Accordingly, an ε-cyclase activity which is reduced compared with the wild type can be determined directly or can take place via determination of the amount of ε-cyclase protein or the amount of ε-cyclase mRNA in the plant of the invention compared with the wild type.

A reduction in ε-cyclase activity comprises a quantitative reduction in an ε-cyclase as far as substantially complete absence of ε-cyclase (i.e. lack of detectability of ε-cyclase activity or lack of immunological detectability of ε-cyclase). The ε-cyclase activity (or the amount of ε-cyclase protein or amount of ε-cyclase mRNA) in the plant, particularly preferably in flowers, is preferably reduced by comparison with the wild type by at least 5%, further preferably by at least 20%, further preferably by at least 50%, further preferably by 100%. “Reduction” means in particular also the complete absence of ε-cyclaseactivity (or of ε-cyclase protein or ε-cyclase mRNA).

Determination of the ε-cyclase activity in genetically modified plants of the invention and in wild-type and reference plants preferably takes place under the following conditions:

The activity of ε-cyclase can be determined in vitro according to Fraser and Sandmann (Biochem. Biophys. Res. Comm. 185(1) (1992) 9-15)), if potassium phosphate as buffer (pH 7.6), lycopene as substrate, paprica stromal protein, NADP+, NADPH and ATP are added to a defined amount of plant extract.

The ε-cyclase activity in genetically modified plants of the invention and in wild-type or reference plants is particularly preferably determined according to Bouvier, d'Harlingue and Camara (Molecular Analysis of carotenoid cyclase inhibition; Arch. Biochem. Biophys. 346(1) (1997) 53-64):

The in vitro assay is carried out in a volume of 0.25 ml. The mixture comprises 50 mM potassium phosphate (pH 7.6), various amounts of plant extract, 20 nM lycopene, 0.25 mg of paprica chromoplast stromal protein, 0.2 mM NADP+, 0.2 mM NADPH and 1 mM ATP. NADP/NADPH and ATP are dissolved in 0.01 ml of ethanol with 1 mg of Tween 80 immediately before the addition to the incubation medium. After a reaction time of 60 minutes at 30 C, the reaction is stopped by adding chloroform/methanol (2:1). The reaction products extracted into chloroform are analyzed by HPLC.

An alternative assay with radioactive substrate is described in Fraser and Sandmann (Biochem. Biophys. Res. Comm. 185(1) (1992) 9-15). A further analytical method is described in Beyer, Kröncke and Nievelstein (On the mechanism of the lycopene isomerase/cyclase reaction in Narcissus pseudonarcissus L. chromropast,; J. Biol. Chem. 266(26) (1991) 17072-17078).

The reduction of ε-cyclase activity in plants is preferably effected by at least one of the following methods:

a) Introduction of at least one double-stranded ε-cyclase ribonucleic acid sequence, also called ε-cyclase dsRNA below, or of an expression cassette or expression cassettes ensuring expression thereof. Methods in which the ε-cyclase dsRNA is directed against an ε-cyclase gene (i.e. genomic DNA sequences such as the promoter sequence) or an ε-cyclase transcript (i.e. mRNA sequences) are included.

b) Introduction of at least one ε-cyclase antisense ribonucleic acid sequence, also called ε-cyclase antisense RNA below, or of an expression cassette ensuring expression thereof. Methods in which the ε-cyclase antisense RNA is directed against an ε-cyclase gene (i.e. genomic DNA sequences) or an ε-cyclase gene transcript (i.e. RNA sequences) are included. Also included are α-anomeric nucleic acid sequences.

c) Introduction of at least one ε-cyclase-antisense RNA combined with a ribozyme or with an expression cassette ensuring expression thereof.

d) Introduction of at least one ε-cyclase sense ribonucleic acid sequence, also called ε-cyclase sense RNA, to induce cosuppression or of an expression cassette ensuring expression thereof.

e) Introduction of at least one DNA- or protein-binding factor against an ε-cyclase gene, RNA or protein or of an expression cassette ensuring expression thereof.

f) Introduction of at least one viral nucleic acid sequence which brings about ε-cyclase RNA degradation, or of an expression cassette ensuring expression thereof.

g) Introduction of at least one construct to produce a loss of function, such as, for example, generation of stop codons or a shifts in the reading frame, in an ε-cyclase gene, for example by producing an insertion, deletion, inversion or mutation in an ε-cyclase gene. It is possible and preferred for knockout mutants to be generated by targeted insertion into said ε-cyclase gene by homologous recombination or introduction of sequence-specific nucleases against ε-cyclase gene sequences.

The skilled worker is aware that other methods can also be employed in the framework of the present invention to diminish an ε-cyclase or its activity or function. It may, for example, also be advantageous to introduce a dominant negative variant of an ε-cyclase or an expression cassette ensuring expression thereof. It is moreover possible for each one of these methods to bring about a diminution in the amount of protein, amount of mRNA and/or activity of an ε-cyclase. Combined use is also conceivable. Further methods are known to the skilled worker and may comprise impeding or suppressing the processing of the ε-cyclase, the transport of the ε-cyclase or its mRNA, inhibition of ribosome attachment, inhibition of RNA splicing, induction of an ε-cyclase RNA-degrading enzyme and/or inhibition of translation elongation or termination.

The individual preferred methods may be described as a consequence by exemplary embodiments:

a) Introduction of a Double-Stranded ε-Cyclase Ribonucleic Acid Sequence ε-Cyclase dsRNA)

The method of gene regulation using double-stranded RNA (“double-stranded RNA interference”; dsRNAi) is known and described for example in Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035 or WO 00/63364. The processes and methods described in the cited documents are incorporated herein by reference.

Under “double-stranded ribonucleic acid sequence” will according to the invention one or more ribonucleic acid sequences which theoretically, for example according to the base-pair rules of Waston and Crick, and/or actually, for example on the basis of hybridization experiments, are able, owing to complementary sequences, to form in vitro and/or in vivo double-stranded RNA structures.

The skilled person is aware that the formation of double-stranded RNA structures represents a dynamic equilibrium. The ratio of double-stranded molecules to corresponding dissociated forms is preferably at least 1 to 10, preferably 1:1, particularly preferably 5:1, most preferably 10:1.

A double-stranded ε-cyclase ribonucleic acid sequence or else ε-cyclase dsRNA preferably means an RNA molecule which has a region with double-stranded structure and comprises in this region a nucleic acid sequence which

a) is identical to at least part of the plant's own ε-cyclase transcript and/or
b) is identical to at least part of the plant's own ε-cyclase promoter sequence.

In the process of the invention, the ε-cyclase activity is reduced preferably by introducing into the plant an RNA which has a region with double-stranded structure and comprises in this region a nucleic acid sequence which

a) is identical to at least part of the plant's own ε-cyclase transcript and/or
b) is identical to at least part of the plant's own ε-cyclase promoter sequence.

The term “ε-cyclase transcript” means that part of an ε-cyclase gene which is transcribed and which, besides the ε-cyclase encoding sequence, for example also comprises non-coding sequences such as, for example, also UTRs.

An RNA which “is identical to at least part of the plant's own ε-cyclase promoter sequence” preferably means that the RNA sequence is identical to at least part of the theoretical transcript of the ε-cyclase promoter sequence, i.e. the corresponding RNA sequence.

“A part” of the plant's own ε-cyclase transcript or of the plant's own ε-cyclase promoter sequence means partial sequences which may extend from a few base pairs up to complete sequences of the transcript or of the promoter sequence. The optimal length of the partial sequences can easily be ascertained by the skilled worker in routine tests.

The length of the partial sequences ordinarily amounts to at least 10 bases and at most 2 kb, preferably at least 25 bases and at most 1.5 kb, particularly preferably at least 50 bases and at most 600 bases, very particularly preferably at least 100 bases and at most 500, most preferably at least 200 bases or at least 300 bases and at most 400 bases.

The partial sequences are preferably selected so that maximal specificity is achieved and activities of other enzymes are not reduced when diminution thereof is undesired. It is therefore advantageous for the chosen partial sequences of the ε-cyclase dsRNA to be parts of the ε-cyclase transcript and/or partial sequences of the ε-cyclase promoter sequences which do not occur in other activities.

In a particularly preferred embodiment, therefore, the ε-cyclase dsRNA comprises a sequence which is identical to a part of the plant's own ε-cyclase transcript and comprises the 5′ end or the 3′ end of the plant's own nucleic acid encoding an ε-cyclase. Particularly suitable for preparing selective double-stranded structures are untranslated regions in the 5′ or 3′ of the transcript.

A further aspect of the invention relates to double-stranded RNA molecules (dsRNA molecules) which, when introduced into a plant organism (or a cell, tissue, organ or propagation material derived therefrom), bring about a diminution in an ε-cyclase.

A double-stranded RNA molecule for reducing the expression of an ε-cyclase (ε-cyclase dsRNA) in this case preferably comprises

a) a sense RNA strand comprising at least one ribonucleotide sequence which is substantially identical to at least a part of the sense RNA ε-cyclase transcript, and
b) an antisense RNA strand which is substantially, preferably completely, complementary to the RNA sense strand in a).

The plant is transformed with an ε-cyclase dsRNA preferably by using a nucleic acid construct which is introduced into the plant and which is transcribed in the plant into the ε-cyclase dsRNA.

The present invention therefore also relates to a nucleic acid construct which can be transcribed into

a) a sense RNA strand comprising at least one ribonucleotide sequence which is substantially identical to at least a part of a sense RNA ε-cyclase transcript, and
b) an antisense RNA strand which is substantially, preferably completely, complementary to the RNA sense strand in a).

These nucleic acid constructs are also called expression cassettes or expression vectors hereinafter.

In relation to the dsRNA molecules, ε-cyclase nucleic acid sequence or the corresponding transcript preferably means the sequence shown in SEQ ID NO: 38 or a part thereof.

“Substantially identical” means that the dsRNA sequence may also have insertions, deletions and single point mutations by comparison with the ε-cyclase target sequence and nevertheless brings about an efficient diminution in expression. The homology is preferably at least 75%, preferably at least 80%, very particularly preferably at least 90%, most preferably 100% between the sense strand of an inhibitory dsRNA and at least one part of the sense RNA transcript of an ε-cyclase gene, or between the antisense strand to the complementary strand of an ε-cyclase gene.

A 100% sequence identity between dsRNA and an ε-cyclase gene transcript is not absolutely necessary to bring about an efficient diminution in ε-cyclase expression. There is accordingly the advantage that the process is tolerant of sequence differences like those which may be present as a result of genetic mutations, polymorphisms or evolutionary divergences. It is thus possible for example with the dsRNA generated starting from the ε-cyclase sequence of one organism to suppress the ε-cyclase expression in another organism. For this purpose, the dsRNA preferably comprises sequence regions of ε-cyclase gene transcripts which correspond to conserved regions. Said conserved regions can easily be inferred from sequence comparisons.

Alternatively, a “substantially identical” dsRNA can also be defined as nucleic acid sequence which is able to hybridize with a part of an ε-cyclase gene transcript (e.g. in 400 mM NaCl, 40 mM PIPES pH 6.4, 1 mM EDTA at 50° C. or 70° C. for 12 to 16 h).

“Substantially complementary” means that the antisense RNA strand may also have insertions, deletions and single point mutations by comparison with the complement of the sense RNA strand. The homology is preferably at least 80%, preferably at least 90%, very particularly preferably at least 95%, most preferably 100% between the antisense RNA strand and the complement of the sense RNA strand.

In a further embodiment, the ε-cyclase dsRNA comprises

a) a sense RNA strand comprising at least one ribonucleotide sequence which is substantially identical to at least a part of the sense RNA transcript of the promoter region of an ε-cyclase gene, and
b) an antisense RNA strand which is substantially—preferably completely—complementary to the RNA sense strand in a).

The corresponding nucleic acid construct which is preferably to be used for transformation of the plants comprises

a) a sense DNA strand which is substantially identical to at least a part of the promoter region of an ε-cyclase gene, and
b) an antisense DNA strand which is substantially—preferably completely—complementary to the DNA sense strand in a).

The promoter region of an ε-cyclase preferably means a sequence as shown in SEQ ID NO: 51 or a part thereof.

The ε-cyclase dsRNA sequences for reducing the ε-cyclase activity are prepared, in particular for Tagetes erecta, particularly preferably by using the following partial sequences:

SEQ ID NO: 52: Sense fragment of the 5′ terminal region of the ε-cyclase

SEQ ID NO: 53: Antisense fragment of the 5′ terminal region of the ε-cyclase

SEQ ID NO: 54: Sense fragment of the 3′ terminal region of the ε-cyclase

SEQ ID NO: 55: Antisense fragment of the 3′ terminal region of the ε-cyclase

SEQ ID NO: 56: Sense fragment of the ε-cyclase promoter

SEQ ID NO: 57: Antisense fragment of the ε-cyclase promoter

The dsRNA may consist of one or more strands of polyribonucleotides. It is, of course, possible to achieve the same purpose by introducing a plurality of individual dsRNA molecules, each of which comprises one of the ribonucleotide sequence segments defined above, into the cell or the organism.

The double-stranded dsRNA structure can be formed starting from two complementary separate RNA strands or—preferably—starting from a single, self-complementary RNA strand. In this case, sense RNA strand and antisense RNA strand are preferably covalently connected together in the form of an inverted repeat.

As described for example in WO 99/53050, the dsRNA may also comprise a hairpin structure where sense and antisense strands are connected by a connecting sequence (linker; for example an intron). The self-complementary dsRNA structures are preferred because they require only the expression of one RNA sequence and comprise the complementary RNA strands always in an equimolar ratio. The connecting sequence is preferably an intron (e.g. an intron of the ST-LS1 gene from potato; Vancanneyt G F et al. (1990) Mol Gen Genet 220(2):245-250).

The nucleic acid sequence coding for a dsRNA may comprise further elements such as, for example, transcription termination signals or polyadenylation signals.

However, if the dsRNA is directed against the promoter sequence of an ε-cyclase, it preferably comprises no transcription termination signals or polyadenylation signals. This makes it possible for the dsRNA to be retained in the nucleus of the cell and prevents the dsRNA being distributed in the whole plant “spreading”). If the two strands of the dsRNA are to be put together in a cell or plant, this can take place by way of example in the following manner:

a) transformation of the cell or plant with a vector which comprises both expression cassettes,

b) cotransformation of the cell or plant with two vectors, where one comprises the expression cassettes with the sense strand and the other comprises the expression cassettes with the antisense strand.

c) crossing of two individual plant lines, where one comprises the expression cassettes with the sense strand and the other comprises the expression cassettes with the antisense strand.

Formation of the RNA duplex can be initiated either outside the cell or inside it.

The dsRNA can be synthesized either in vivo or in vitro. For this purpose, a DNA sequence coding for a dsRNA can be put into an expression cassette under the control of at least one genetic control element (such as, for example, a promoter). Polyadenylation is unnecessary, nor need any elements for initiating translation be present. The expression cassette for the MP dsRNA is preferably present on the transformation construct or the transformation vector.

In a particularly preferred embodiment, expression of the dsRNA takes place starting from an expression construct under the functional control of a flower-specific promoter.

The expression cassettes coding for the antisense and/or the sense strand of an ε-cyclase dsRNA or for the self-complementary strand of the dsRNA are for this purpose preferably inserted into a transformation vector and introduced by the methods described below into the plant cell. Stable insertion into the genome is advantageous for the process of the invention.

The dsRNA can be introduced in an amount which makes at least one copy per cell possible. Larger amounts (e.g. at least 5,10,100, 500 or 1000 copies per cell) may where appropriate bring about an efficient diminution.

b) Introduction of an Antisense Ribonucleic Acid Sequence of an ε-Cyclase (ε-Cyclase Antisense RNA)

Methods for diminishing a particular protein by antisense technology have been described many times—also in plants (Sheehy et al. (1988) Proc Natl Acad Sci USA 85: 8805-8809; U.S. Pat. No. 4,801,340; Mol J N et al. (1990) FEBS Lett 268(2):427-430). The antisense nucleic acid molecule hybridizes or binds to the cellular mRNA and/or genomic DNA coding for the ε-cyclase to be diminished. The transcription and/or translation of the ε-cyclase is suppressed thereby. The hybridization can arise in a conventional manner via formation of a stable duplex or—in the case of genomic DNA—through binding of the antisense nucleic acid molecule to the duplex of genomic DNA by specific interaction in the major groove of the DNA helix.

An ε-cyclase antisense RNA can be inferred by using nucleic acid sequence coding for this ε-cyclase, for example the nucleic acid sequence as shown in SEQ ID NO: 58, according to the base-pair rules of Watson and Crick. The ε-cyclase antisense RNA may be complementary to the entire transcribed mRNA of the ε-cyclase, be confined to the coding region or consist only of one oligonucleotide which is complementary to a part of the coding or noncoding sequence of the mRNA. Thus, the oligonucleotide may for example be complementary to the region which comprises the start of ε-cyclase translation. The ε-cyclase antisense RNA may have a length of, for example 5,10, 15, 20, 25, 30, 35, 40, 45 or 50 nucleotides, but may also be longer and comprise at least 100, 200, 500, 1000, 2000 or 5000 nucleotides. ε-Cyclase antisense RNAs are preferably expressed recombinantly in the target cell within the framework of the process of the invention.

A further aspect of the invention relates to transgenic expression cassettes comprising a nucleic acid sequence coding for at least a part of an ε-cyclase, where said nucleic acid sequence, where said nucleic acid sequence is functionally linked to a promoter functional in plant organisms in antisense orientation. In a particularly preferred embodiment, the expression of the antisense RNA takes place starting from an expression construct under functional control of a flower-specific promoter.

Said expression cassettes may be part of a transformation construct or transformation vector, or else be introduced within the framework of a cotransformation.

In a further preferred embodiment, expression of an ε-cyclase can be inhibited by nucleotide sequences which are complementary to the regulatory region of an ε-cyclase gene (e.g. an ε-cyclase promoter and/or enhancer) and form triple-helical structures with the DNA double helix there, so that transcription of the ε-cyclase gene is reduced. Corresponding methods are described (Helene C (1991) Anticancer Drug Res 6(6):569-84; Helene C et al. (1992) Ann NY Acad Sci 660:27-36; Maher U (1992) Bioassays 14(12):807-815).

In a further embodiment, the ε-cyclase antisenseRNA may be an α-anomeric nucleic acid. Such α-anomeric nucleic acid molecules form specific double-stranded hybrids with complementary RNA in which—differing from conventional β-nucleic acids—the two strands run parallel to one another (Gautier C et al. (1987) Nucleic Acids Res 15:6625-6641).

c) Introduction of an ε-Cyclase Antisense RNA Combined with a Ribozyme

It is possible and advantageous to couple the antisense strategy described above with a ribozyme method. Catalytic RNA molecules or ribozymes can be adapted to any target RNA and cleave the phosphodiester structure at specific positions, thus functionally deactivating the target RNA (Tanner N K (1999) FEMS Microbiol Rev 23(3):257-275). The ribozyme itself is not modified thereby, but is able to cleave further target RNA molecules analogously, thus retaining the properties of an enzyme. Incorporation of ribozyme sequences into antisense RNAs confers this enzyme-like, RNA-cleaving property on precisely these antisense RNAs and thus increases the efficiency thereof in the inactivation of target RNA. The preparation and use of corresponding ribozyme-antisense RNA molecules is described (inter alia in Haseloff et al. (1988) Nature 334: 585-591); Haselhoff and Gerlach (1988) Nature 334:585-591; Steinecke P et al. (1992) EMBO J 11(4):1525-1530; de Feyter R et al. (1996) Mol Gen Genet. 250(3):329-338).

It is possible in this way to use ribozymes (e.g. hammerhead ribozymes; Haselhoff and Gerlach (1988) Nature 334:585-591) for catalytic cleavage of the mRNA of an ε-cyclase to be diminished, and thus for prevention of translation. The ribozyme technology may increase the efficiency of an antisense strategy. Methods for the expression of ribozymes for diminishing certain proteins are described in (EP 0 291 533, EP 0 321 201, EP 0 360 257). Ribozyme expression in plant cells has likewise been described (Steinecke P et al. (1992) EMBO J 11(4):1525-1530; de Feyter R et al. (1996) Mol Gen Genet. 250(3):329-338). Suitable target sequences and ribozymes can be determined for example as described in “Steinecke P, Ribozymes, Methods in Cell Biology 50, Galbraith et al. eds, Academic Press, Inc. (1995), pp. 449-460”, by calculations of secondary structures of ribozyme and target RNA, and by the interaction thereof (Bayley C C et al. (1992) Plant Mol Biol. 18(2):353-361; Lloyd A M. and Davis R W et al. (1994) Mol Gen Genet. 242(6):653-657). For example, derivatives of the tetrahymena L-19 IVS RNA which have complementary regions to the mRNA of the ε-cyclase to be suppressed can be constructed (see also U.S. Pat. No. 4,987,071 and U.S. Pat. No. 5,116,742). It is possible alternatively to identify such ribozymes by a selection process from a library of diverse ribozymes (Bartel D and Szostak J W (1993) Science 261:1411-1418).

d) Introduction of a Sense Ribonucleic Acid Sequence of an ε-Cyclase (ε-Cyclase Sense RNA) for Inducing a Cosuppression.

The expression of an ε-cyclase ribonucleic acid sequence (or of a part thereof) in sense orientation may lead to cosuppression of the corresponding ε-cyclase gene.

Expression of sense RNA having homology to an endogenous ε-cyclase gene may diminish or abolish expression thereof, in a similar way to that described for antisense approaches (Jorgensen et al. (1996) Plant Mol Biol 31(5):957-973; Goring et al. (1991) Proc Natl Acad Sci USA 88:1770-1774; Smith et al. (1990) Mol Gen Genet 224:447-481; Napoli et al. (1990) Plant Cell 2:279-289; Van der Krol et al. (1990) Plant Cell 2:291-99). Here, the construct to be introduced can represent the homologous gene to be diminished wholly or only in part. The possibility of translation is unnecessary. Application of this technology to plants is described (e.g. Napoli et al. (1990) Plant Cell 2:279-289; in U.S. Pat. No. 5,034,323.

The cosuppression is preferably implemented using a sequence which is substantially identical to at least a part of the nucleic acid sequence coding for an ε-cyclase, for example of the nucleic acid sequence shown in SEQ ID NO: 38.

The ε-cyclase sense RNA is preferably chosen so that translation of the ε-cyclase or of a part thereof cannot occur. It is possible for this purpose for example to choose the 5′-untranslated or 3′-untranslated region or else to delete or mutate the ATG start codon.

e) Introduction of DNA- or Protein-Binding Factors Against ε-Cyclase Genes, RNAs or Proteins

A diminution in ε-cyclase expression is also possible with specific DNA-binding factors, e.g. with factors of the type of zinc finger transcription factors. These factors attach themselves to the genomic sequence of the endogenous target gene, preferably in the regulatory regions, and bring about a diminution in expression. Appropriate methods for preparing corresponding factors are described (Dreier B et al. (2001) J Biol Chem 276(31):29466-78; Dreier B et al. (2000) J Mol Biol 303(4):489-502; Beerli R R et al. (2000) Proc Natl Acad Sci USA 97 (4):1495-1500; Beerli R R et al. (2000) J Biol Chem 275(42):32617-32627; Segal D J and Barbas C F 3rd. (2000) Curr Opin Chem Biol 4(1):34-39; Kang J S and Kim J S (2000) J Biol Chem 275(12):8742-8748; Beerli R R et al. (1998) Proc Natl Acad Sci USA 95(25):14628-14633; Kim J S et al. (1997) Proc Natl Acad Sci USA 94(8):3616-3620; Klug A (1999) J Mol Biol 293(2):215-218; Tsai S Y et al. (1998) Adv Drug Deliv Rev 30(1-3):23-31; Mapp A K et al. (2000) Proc Natl Acad Sci USA 97(8):3930-3935; Sharrocks A D et al. (1997) Int J Biochem Cell Biol 29(12):1371-1387; Zhang L et al. (2000) J Biol Chem 275(43):33850-33860).

The selection of these factors can take place using any piece of an ε-cyclase gene. This segment is preferably located in the region of the promoter region. However, for gene suppression, it may also be located in the region of coding exons or introns.

A further possibility is to introduce into a cell factors which themselves inhibit the ε-cyclase. These protein-binding factors may be for example aptamers (Famulok M and Mayer G (1999) Curr Top Microbiol Immunol 243:123-36) or antibodies or antibody fragments or single-chain antibodies. The isolation of these factors is described (Owen M et al. (1992) Biotechnology (N Y) 10(7):790-794; Franken E et al. (1997) Curr Opin Biotechnol 8(4):411-416; Whitelam (1996) Trend Plant Sci 1:286-272).

f) Introduction of Viral Nucleic Acid Sequences and Expression Constructions which Bring about ε-Cyclase RNA Degradation

ε-cyclase expression can also be efficiently implemented by inducing specific ε-cyclase RNA degradation by the plant with the aid of a viral expression system (amplicon; Angell S M et al. (1999) Plant J 20(3):357-362). These systems—also referred to as VIGS (viral induced gene silencing)—introduce nucleic acid sequences having homology to the transcript of an ε-cyclase to be diminished into the plant by means of viral vectors. Transcription is then abolished—presumably mediated by the plant's defence mechanisms against viruses. Corresponding techniques and methods are described (Ratcliff F et al. (2001) Plant J 25(2):237-45; Fagard M and Vaucheret H (2000) Plant Mol Biol 43(2-3):285-93; Anandalakshmi R et al. (1998) Proc Natl Acad Sci USA 95(22):13079-84; Ruiz M T (1998) Plant Cell 10(6):937-46).

The VIGS-mediated diminution is preferably implemented by using a sequence which is substantially identical to at least a part of the nucleic acid sequence coding for an ε-cyclase, for example the nucleic acid sequence shown in SEQ ID NO: 1.

g) Introduction of Constructs to Produce a Loss of Function or a Diminution of Function of ε-Cyclase Genes

The skilled worker is aware of numerous methods allowing targeted modification of genomic sequences. These include in particular methods such as the production of knockout mutants by means of targeted homologous recombination, e.g. by generating stop codons, shifts in the reading frame etc. (Hohn B and Puchta H (1999) Proc Natl Acad Sci USA 96:8321-8323) or targeted deletion or inversion of sequences by means of, for example, sequence-specific recombinases or nucleases (see below)

The amount, function and/or activity of ε-cyclase can also be diminished by a targeted insertion of nucleic acid sequences (e.g. of the nucleic acid sequence to be inserted within the framework of the process invention) into the sequence coding for an ε-cyclase (e.g. by intermolecular homologous recombination). In this embodiment there is preferably use of a DNA construct which comprises at least one part of the sequence of an ε-cyclase gene or adjacent sequences, and is thus capable of targeted recombination therewith in the target cell, so that the ε-cyclase gene is so modified by a deletion, addition or substitution of at least one nucleotide that the functionality of the ε-cyclase gene is reduced or entirely abolished. The modification may also affect the regulatory elements (e.g. the promoter) of the ε-cyclase gene, so that the coding sequence remains unmodified, but expression (transcription and/or translation) does not occur and is reduced. In conventional homologous recombination, the sequence to be inserted is flanked at its 5′ and/or 3′ end by further nucleic acid sequences (A′ and B′ respectively) which have a sufficient length and homology to corresponding sequences of the ε-cyclase gene (A and B, respectively) to enable homologous recombination. The length is usually in a range from several hundred bases up to several kilobases (Thomas K R and Capecchi M R (1987) Cell 51:503; Strepp et al. (1998) Proc Natl Acad Sci USA 95(8):4368-4373). For the homologous recombination, the plant cell is transformed with the recombination construct using the methods described below, and successfully recombined clones are selected on the basis of the consequently inactivated ε-cyclase.

In a further preferred embodiment, the efficiency of recombination is increased by combination with methods which promote homologous recombination. Such methods are described and comprise for example the expression of proteins such as RecA or the treatment with PARP inhibitors. It has been possible to show that intrachromosomal homologous recombination in tobacco plants can be increased through the use of PARP inhibitors (Puchta H et al. (1995) Plant J 7:203-210). It is possible by using these inhibitors to increase further the rate of homologous recombination in the recombination constructs after induction of the sequence specific DNA double-strand break and thus the efficiency of deletion of the transgenic sequences. Various PARP inhibitors can be employed in this connection. Preferably included are inhibitors such as 3-aminobenzamide, 8-hydroxy-2-methylquinazolin-4-one (NU 1025), 1,11b-dihydro-[2H]benzopyrano[4,3,2-de]isoquinolin-3-one (GPI 6150), 5-aminoisoquinolinone, 3,4-dihydro-5-[4-(1-piperidinyl)butoxy]-1 (2H)-isoquinolinone or the substances described in WO 00/26192, WO 00/29384, WO 00/32579, WO 00/64878, WO 00/68206, WO 00/67734, WO 01/23386 and WO 01/23390.

Further suitable methods are the introduction of nonsense mutations into endogenous marker protein genes, for example by introducing RNA/DNA oligonucleotides into the plant (Zhu et al. (2000) Nat Biotechnol 18(5):555-558) or the generation of knockout mutants by means of, for example, T-DNA mutagenesis (Koncz et al., Plant Mol. Biol. 1992, 20(5):963-976). Point mutations can also be produced by means of DNA-RNA hybrids which are also known as “chimeraplasty” (Cole-Strauss et al. (1999) Nucl Acids Res 27(5):1323-1330; Kmiec (1999) Gene therapy American Scientist 87(3):240-247).

The methods of dsRNAi, of the cosuppression using sense RNA and of “VIGS” (“virus induced gene silencing”) are also referred to as post-transcriptional gene silencing (PTGS) or transcriptional gene silencing (TGS). PTGS/TGS methods are particularly advantageous because of the lower requirements for the homology between the marker protein gene to be diminished and the transgenically expressed sense or dsRNA nucleic acid sequence than in the case of, for example, a conventional antisense approach. Thus, use of the marker protein nucleic acid sequences from one species can also efficiently diminish the expression of homologous marker protein proteins in other species without the absolute necessity for isolation and elucidation of the structure of the marker protein homologs occurring there. This considerably lightens the workload.

In a particularly preferred embodiment of the process of the invention, the reduction of ε-cyclase activity compared with the wild type is effected by:

a) introduction of at least one double-stranded ε-cyclase ribonucleic acid sequence or of an expression cassette or expression cassettes ensuring expression thereof into plants and/or
b) introduction of at least one ε-cyclase antisense ribonucleic acid sequences or of an expression cassette ensuring expression thereof into plants.

In a very particularly preferred embodiment, reduction of ε-cyclase activity compared with the wild type is effected by introduction of at least one double-stranded ε-cyclase ribonucleic acid sequence or of an expression cassette or expression cassettes ensuring expression thereof into plants.

In a preferred embodiment, genetically modified plants which have the lowest expression rate of an ε-cyclase in flowers are used.

This is preferably achieved by reducing the ε-cyclase activity flower-specifically, particularly preferably petal-specifically.

In the particularly preferred embodiment described above, this is achieved by the transcription of the ε-cyclase dsRNA sequences taking place under the control of a flower-specific promoter or, even more preferably, under the control of a petal-specific promoter.

Particularly preferred plants are plants selected from the families of Amaranthaceae, Amaryllidaceae, Apocynaceae, Asteraceae, Balsaminaceae, Begoniaceae, Berberidaceae, Brassicaceae, Cannabaceae, Caprifoliaceae, Caryophyliaceae, Chenopodiaceae, Compositae, Cucurbitaceae, Cruciferae, Euphorbiaceae, Fabaceae, Gentianaceae, Geraniaceae, Graminae, Illiaceae, Labiatae, Lamiaceae, Leguminosae, Liliaceae, Linaceae, Lobeliaceae, Malvaceae, Oleaceae, Orchidaceae, Papaveraceae, Plumbaginaceae, Poaceae, Polemoniaceae, Primulaceae, Ranunculaceae, Rosaceae, Rubiaceae, Scrophulariaceae, Solanaceae, Tropaeolaceae, Umbelliferae, Verbanaceae, Vitaceae and Violaceae.

Very particularly preferred plants are selected from the group of plant genera Marigold, Tagetes errecta, Tagetes patula, Acacia, Aconitum, Adonis, Arnica, Aquilegia, Aster, Astragalus, Bignonia, Calendula, Caltha, Campanula, Canna, Centaurea, Chemanthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, particularly preferably selected from the group of plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

In the process of the invention for producing ketocarotenoids, the step of cultivating the genetically modified organisms is preferably followed by harvesting of the organisms and further preferably additionally by isolation of ketocarotenoids from the organisms.

The harvesting of the organisms takes place in a manner known per se and appropriate for the particular organism. Microorganisms such as bacteria, yeasts, algae or fungi or plant cells which are cultivated by fermentation in liquid nutrient media can be removed for example by centrifugation, decantation or filtration. Plants are grown in a manner know per se on nutrient media and harvested appropriately.

The genetically modified microorganims are preferably cultivated in the presence of oxygen at a cultivation temperature of at least about 20° C., such as, for example, 20° C. to 40° C., and at a pH of about 6 to 9. In the case of genetically modified microorganisms, the microorganisms are preferably initially cultivated in the presence of oxygen and in a complex medium such as, for example TB or LB medium at a cultivation temperature of about 20° C. or more, and at a pH of about 6 to 9, until a sufficient cell density is reached. To enable better control of the oxidation reaction, it is preferred to use an inducible promoter. Cultivation is continued after induction of ketolase expression in the presence of oxygen for from 12 hours to 3 days, for example.

The ketocarotenoids are isolated from the harvested biomass in a manner known per se, for example by extraction and, if appropriate, further chemical or physical purification processes such as, for example, precipitation methods, crystallography, thermal separation methods such as rectification methods or physical separation methods such as, for example chromatography.

As mentioned below, the ketocarotenoids can be specifically produced in the genetically modified plants of the invention preferably in various plant tissues such as for example, seeds, leaves, fruits, flowers, especially in petals.

Ketocarotenoids are isolated from the harvested petals in a manner known per se, for example by drying and subsequent extraction and, if appropriate, further chemical or physical purification processes such as, for example, precipitation methods, crystallography, thermal separation methods such as rectification methods or physical separation methods such as, for example, chromatography. Ketocarotenoids are isolated from the petals for example preferably by organic solvents such as acetone, hexane, ether or tert-methyl butyl ether.

Further methods for isolating ketocarotenoids, especially from petals, are described for example in Egger and Kleinig (Phytochemistry (1967) 6, 437-440) and Egger (Phytochemistry (1965) 4, 609-618).

The ketocarotenoids are preferably selected from the group of astaxanthin, canthaxanthin, echinenone, 3-hydroxyechinenone, 3′-hydroxyechinenone, adonirubin and adonixanthin.

A particularly preferred ketocarotenoid is astaxanthin.

Depending on the organism used, the ketocarotenoids result in free form or as fatty acid esters or as diglucosides.

In the process of the invention, the ketocarotenoids result in the petals of plants in the form of their mono- and diesters with fatty acids. Examples of some detected fatty acids are myristic acid, palmitic acid, stearic acid, oleic acid, linoleic acid and lauric acid (Kamata and Simpson (1987) Comp. Biochem. Physiol Vol. 86B(3), 587-591).

The ketocarotenoids can be produced in the whole plant or, in a preferred embodiment, specifically in plant tissues which comprise chromoplasts. Examples of preferred plant tissues are roots, seeds, leaves, fruits, flowers and, especially, nectaries and petals.

In a further, particularly preferred embodiment of the process of the invention, genetically modified plants which exhibit the highest expression rate of a ketolase in fruits are used.

This is preferably achieved by expression of the ketolase gene taking place under the control of a fruit-specific promoter. For this purpose, for example, the nucleic acids described above are introduced, as described in detail below, in a nucleic acid construct functionally linked to a fruit-specific promoter into the plant. In a further, particularly preferred embodiment of the process of the invention, genetically modified plants which exhibit the highest expression rate of a ketolase in seeds are used.

This is preferably achieved by expression of the ketolase gene taking place under the control of a seed-specific promoter. For this purpose, for example, the nucleic acids described above are introduced, as described in detail below, in a nucleic acid construct functionally linked to a seed-specific promoter into the plant.

The targeting into the chromplasts is effected by a functionally linked plastid transit peptide.

The production of genetically modified plants with raised or caused ketolase activity is described by way of example below, where altered ketolase activity is caused by a ketolase selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10.
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

Further activities such as, for example, β-cyclase activity, hydroxylase activity, HMG-CoA reductase activity, (E)-4-hydroxy-3-methylbut-2-enyl-diphosphate reductase activity, 1-deoxy-D-xylose-5-phosphate synthase activity, 1-deoxy-D-xylose-5-phosphate reductoisomerase activity, isopentenyl-diphosphate Δ-isomerase activity, geranyl-diphosphate synthase activity, farnesyl-diphosphate synthase activity, geranylgeranyl-diphosphate synthase activity, phytoene synthase activity, phytoene desaturase activity, zeta-carotene desaturase activity, crtISO activity, FtsZ activity and/or MinD activity can be raised analogously by using the appropriate effect genes.

The transformation can in the case of combinations of genetic modifications take place singly or through multiple constructs.

The transgenic plants are preferably produced by transformation of the initial plants with a nucleic acid construct which comprises at least one of the effect genes described above, which is functionally linked to one or more regulatory signals which ensure transcription and translation in plants.

These nucleic acid constructs in which the effect genes are functionally linked to one or more regulatory signals which ensure transcription and translation in plants are also called expression cassettes hereinafter.

The regulatory signals preferably comprise one or more promoters which ensure transcription and translation in plants.

The expression cassettes comprise regulatory signals, i.e. regulatory nucleic acid sequences, which control the expression of the effect genes in the host cell. In a preferred embodiment, an expression cassette comprises upstream, i.e. at the 5′ end of the coding sequence, a promoter and downstream, i.e. at the 3′ end, a polyadenylation signal and, if appropriate, further regulatory elements which are operatively linked to the coding sequence, lying between them, of the effect gene for at least one of the genes described above. An operative linkage means the sequential arrangement of promoter, coding sequence, terminator and, if appropriate, further regulatory elements in such a way that each of the regulatory elements is able to perform its function as intended as expression of the coding sequence.

By way of example hereinafter the preferred nucleic acid constructs, expression cassettes and vectors for plants and processes for producing transgenic plants, and the transgenic plants themselves, are described.

The sequences which are preferred for the operative linkage but which are not restricted thereto are targeting sequences for ensuring subcellular localization in the apoplast, in the vacuole, in plastids, in the mitochondrion, in the endoplasmic reticulum (ER), in the cell nucleus, in elaioplasts or other compartments and translation enhancers such as the 5′ leader sequence from the tobacco mosaic virus (Gallie et al., Nucl. Acids Res. 15 (1987), 8693-8711).

The promoter suitable for the expression cassette is in principle any promoter which can control the expression of foreign genes in plants.

“Constitutive” promoter means those promoters which ensure expression in numerous, preferably all, tissues over a relatively long period of the development of the plant, preferably at all times during the development of the plant.

Particular preference is given to the use of a plant promoter or a promoter derived from a plant virus. Particular preference is given to the promoter of the 35S transcript of the CaMV cauliflower mosaic virus (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228), of the 19S CaMV promoter (U.S. Pat. No. 5,352,605; WO 84/02913; Benfey et al. (1989) EMBO J. 8:2195-2202), the triose phosphate translocator (TPT) promoter from Arabidopsis thaliana Acc. No. AB006698, base pair 53242 to 55281; the gene starting at bp 55282 is annotated “phosphate/triose-phosphate translocator”, or the 34S promoter from figwort mosaic virus Acc. No. X16673, base pair 1 to 554.

A further suitable constitutive promoter is the pds promoter (Pecker et al. (1992) Proc. Natl. Acad. Sci. USA 89: 4962-4966) or the Rubisco small subunit (SSU) promoter (U.S. Pat. No. 4,962,028), the leguminB promoter (GenBank Acc. No. X03677), the agrobacterium nopaline synthase promoter, the TR double promoter, the agrobacterium OCS (octopine synthase) promoter, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits or the promoter of a proline-rich protein from wheat (WO 91/13991), the Pnit promoter (Y07648.L, Hillebrand et al. (1998), Plant. Mol. Biol. 36, 89-99, Hillebrand et al. (1996), Gene, 170, 197-200) and further promoters of genes whose constitutive expression in plants is known to the skilled worker.

The expression cassettes may also comprise a chemically inducible promoter (review article: Gatz et al. (1997) Annu Rev Plant Physiol Plant Mol Biol 48:89-108), by which the expression of the effect genes in the plant can be controlled at a particular time. Promoters of this type, such as, for example, the PRP1 promoter (Ward et al. (1993) Plant Mol Biol 22:361-366), a salicylic acid-inducible promoter (WO 95/19443), a benzenesulfonamide-inducible promoter (EP 0 388 186), a tetracycline-inducible promoter (Gatz et al. (1992) Plant J 2:397-404), an abscissic acid-inducible promoter (EP 0 335 528) or an ethanol- or cyclohexanone-inducible promoter (WO 93/21334) can likewise be used.

Further preferred promoters are those induced by biotic or abiotic stress, such as, for example, the pathogen-inducible promoter of the PRP1 gene (Ward et al. (1993) Plant Mol Biol 22:361-366), the heat-inducible hsp70 or hsp80 promoter from tomato (U.S. Pat. No. 5,187,267), the cold-inducible alpha-amylase promoter from potato (WO 96/12814), the light-inducible PPDK promoter or the wound-induced pinII promoter (EP375091).

Pathogen-inducible promoters comprise those of genes which are induced as a result of pathogen infestation, such as, for example, genes of PR proteins, SAR proteins, b-1,3-glucanase, chitinase etc. (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich-et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968 (1989).

Also included are wound-inducible promoters such as that of the pinII gene (Ryan (1990) Ann Rev Phytopath 28:425-449; Duan et al. (1996) Nat Biotech 14:494-498), of the wun1 and wun2 gene (U.S. Pat. No. 5,428,148), of the win1 and win2 gene (Stanford et al. (1989) Mol Gen Genet. 215:200-208), of the systemin gene (McGurl et al. (1992) Science 225:1570-1573), of the WIP1 gene (Rohmeier et al. (1993) Plant Mol Biol 22:783-792; Ekelkamp et al. (1993) FEBS Letters 323:73-76), of the MPI gene (Corderok et al. (1994) The Plant J 6(2):141-150) and the like.

Further suitable promoters are, for example, fruit ripening-specific promoters such as, for example, the fruit ripening-specific promoter from tomato (WO 94/21794, EP 409 625). Development-dependent promoters includes some of the tissue-specific promoters, because the formation of individual tissues by its nature takes place in a development-dependent fashion.

Further particularly preferred promoters are those which ensure expression in tissues or plant parts in which, for example, the biosynthesis of ketocarotenoids or its precursors takes place. Preferred examples are promoters with specificities for the anthers, ovaries, petals, sepals, flowers, leaves, stalks, seeds and roots and combinations thereof.

Tuber-, storage root- or root-specific promoters are, for example, the patatin-promoter of class I (B33) or the promoter of the cathepsin D inhibitor from potato.

Leaf-specific promoters are, for example, the promoter of the cytosolic FBPase from potato (WO 97/05900), the SSU promoter (small subunit) of Rubisco (ribulose-1,5-bisphosphate carboxylase) or the ST-LSI promoter from potato (Stockhaus et al. (1989) EMBO J. 8:2445-2451).

Flower-specific promoters are, for example, the phytoene synthase promoter (WO 92/16635) or the promoter of the P-rr gene (WO 98/22593), the AP3 promoter from Arabidopsis thaliana (see Example 5), the CHRC promoter (chromoplast-specific carotenoid-associated protein (CHRC) gene promoter from Cucumis sativus Acc. No. AF099501, base pair 1 to 1532), the EPSP_synthase promoter (5-enolpyruvylshikimate-3-phosphate synthase gene promoter from Petunia hybrida, Acc. No. M37029, base pair 1 to 1788), the PDS promoter (phytoene desaturase gene promoter from Solanum lycopersicum, Acc. No. U46919, base pair 1 to 2078), the DFR-A promoter (dihydroflavonol 4-reductase gene A promoter from Petunia hybrida, Acc.-No. X79723, base pair 32 to 1902) or the FBP1 promoter (floral binding protein 1 gene promoter from Petunia hybrida, Acc. No. L10115, base pair 52 to 1069).

Another-specific promoters are, for example, the 5126 promoter (U.S. Pat. No. 5,689,049, U.S. Pat. No. 5,689,051), the glob-I promoter or the g-zein promoter.

Seed-specific promoters are, for example, the ACP05 promoter (acyl carrier protein gene, WO9218634), the promoters AtS1 and AtS3 of Arabidopsis (WO 9920775), the LeB4 promoter from Vicia faba (WO 9729200 and U.S. Pat. No. 6,403,371), the napin promoter from Brassica napus (U.S. Pat. No. 5,608,152; EP 255378; U.S. Pat. No. 5,420,034), the SBP promoter from Vicia faba (DE 9903432) or the corn promoters End1 and End2 (WO 0011177).

Further promoters suitable for expression in plants are described in Rogers et al. (1987) Meth in Enzymol 153:253-277; Schardl et al. (1987) Gene 61:1-11 and Berger et al. (1989) Proc Natl Acad Sci USA 86:8402-8406).

Constitutive, seed-specific, fruit-specific, flower-specific and, in particular, petal-specific promoters are particularly preferred in the process of the invention.

An expression cassette is prepared preferably by fusing a suitable promoter to at least one of the effect genes described above, and preferably to a nucleic acid which is inserted between promoter and nucleic acid sequence and which codes for a plastid-specific transit peptide, and to a polyadenylation signal by conventional recombination and cloning techniques as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The preferably inserted nucleic acids encoding a plastid transit peptide ensure localization in plastids and especially in chromoplasts.

It is also possible to use expression cassettes whose nucleic acid sequence codes for an effect gene product fusion protein, where one part of the fusion protein is a transit peptide which controls the translocation of the polypeptide. Preference is given to transit peptides which are specific for chromoplasts and which, after translocation of the effect genes into the chromoplasts, are eliminated enzymatically from the effect gene product part.

Particular preference is given to the transit peptide which is derived from the Nicotiana tabacum plastid transketolase or from another transit peptide (e.g. the transit peptide of the small subunit of rubisco (rbcS) or of the ferredoxin NADP oxidoreductase and of the isopentenyl-pyrophosphate isomerase-2) or its functional equivalent.

Special preference is given to nucleic acid sequences from three cassettes of the plastid transit peptide of the plastid transketolase from tobacco in three reading frames as KpnI/BamHI fragments with an ATG codon in the NcoI cleavage site:

pTP09
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGATCC_BamHI
pTP10
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGCTGGATCC_BamHI
pTP11
KPnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGGATCC_BamHI

Further examples of a plastid transit peptide are the transit peptide of the plastid isopentenyl-pyrophosphate isomerase-2 (IPP-2) from Arabisopsis thaliana and the transit peptide of the small subunit of ribulose bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16: 11380).

The nucleic acids of the invention can be prepared synthetically or isolated naturally or comprise a mixture of synthetic and natural nucleic acid constituents, and consist of various heterologous gene segments from different organisms.

Preference is given, as described above, to synthetic nucleotide sequences with codons which are preferred by plants. These codons which are preferred by plants can be determined from codons with the highest protein frequency which are expressed in most of the plant species of interest.

It is possible in the preparation of an expression cassette to manipulate various DNA fragments in order to obtain a nucleotide sequence which expediently reads in the correct direction and which is equipped with a correct reading frame. Adapters or linkers can be attached to the fragments to join the DNA fragments together.

It is expediently possible for the promoter and terminator regions to be provided, in the direction of transcription, with a linker or polylinker comprising one or more restriction sites for the insertion of this sequence. Ordinarily, the linker has 1 to 10, in most cases 1 to 8, preferably 2 to 6, restriction sites. In general, the linker within the regulatory regions has a size of less than 100 bp, frequently less than 60 bp, but at least 5 bp. The promoter can be both native, or homologous, and foreign, or heterologous, with regard to the host plant. The expression cassette preferably comprises, in the 5′-3′ direction of transcription, the promoter, a coding nucleic acid sequence or a nucleic acid construct and a region for termination of transcription. Different termination regions can be mutually exchanged as desired.

Examples of a terminator are the ³⁵S terminator (Guerineau et al. (1988) Nucl Acids Res. 16: 11380), the nos terminator (Depicker A, Stachel S, Dhaese P, Zambryski P, Goodman H M. Nopaline synthase: transcript mapping and DNA sequence. J. Mol Appl Genet. 1982; 1 (6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J. 3: 20 35-846).

It is moreover possible to employ manipulations which provide suitable restriction cleavage sites or which remove surplus DNA or restriction cleavage sites. Where insertions, deletions or substitutions such as, for example, transitions and transversions are suitable, it is possible to use in vitro mutagenesis, primer repair, restriction or ligation.

In the case of suitable manipulations such as, for example, restriction, chewing back or filling in of overhangs for blunt ends, it is possible to provide complementary ends of the fragments for the ligation.

Preferred polyadenylation signals are plant polyadenylation signals, preferably those substantially corresponding to T-DNA polyadenylation signals from Agrobacterium tumefaciens, especially of gene 3 of the T-DNA (octopine synthase) of the Ti plasmid pTiACH5 (Gielen et al., EMBO J. 3 (1984), 835 ff) or functional equivalents.

The transfer of foreign genes into the genome of a plant is referred to as transformation.

It is possible to use for this purpose methods known per se for the transformation and regeneration of plants from plant tissues or plant cells for transient or stable transformation.

Suitable methods for the transformation of plants are protoplast transformation by polyethylene glycol-induced DNA uptake, the biolistic method with the gene gun—the so-called particle bombardment method, electroporation, incubation of dry embryos in DNA-containing solution, microinjection, and the gene transfer mediated by Agrobacterium as described above. Said methods are described for example in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225).

The construct to be expressed is preferably cloned into a vector which is suitable for transformation of Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria transformed with an expression plasmid can be used in a known manner for the transformation of plants, e.g. by bathing wounded leaves or pieces of leaf in a solution of agrobacteria and subsequently cultivating in suitable media.

For the preferred production of genetically modified plants, also referred to as transgenic plants hereinafter, the fused expression cassette is cloned into a vector, for example pBin19 or, in particular, pSUN5 and pSUN3, which is suitable to be transformed into Agrobacterium tumefaciens. Agrobacteria transformed with such a vector can then be used in a known manner for the transformation of plants, in particular of crop plants, by for example bathing wounded leaves or pieces of leaf in a solution of agrobacteria and subsequently cultivating in suitable media.

The transformation of plants by agrobacteria is disclosed inter alia in F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants comprising one or more genes integrated into the expression cassette can be regenerated in a known manner from the transformed cells of the wounded leaves or pieces of leaf.

To transform a host plant with one or more effect genes of the invention, an expression cassette is incorporated as insertion into a recombinant vector whose vector DNA comprises additional functional regulatory signals, for example sequences for replication or integration. Suitable vectors are described inter alia in “Methods in Plant Molecular Biology and Biotechnology” (CRC Press), chapters 6/7, pp. 71-119 (1993). It is possible by using the recombination and cloning techniques quoted above to clone the expression cassettes into suitable vectors which make their replication possible, for example in E. coli. Suitable cloning vectors are inter alia pJIT117 (Guerineau et al. (1988) Nucl. Acids Res.16: 11380), pBR332, pUC series, M13 mp series and pACYC184. Binary vectors able to replicate both in E. coli and in agrobacteria are particularly suitable.

The production of genetically modified microorganisms of the invention having raised or caused ketolase activity is described by way of example hereinafter where the altered ketolase activity is caused by a ketolase selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

Further activities such as, for example, β-cyclase activity, hydroxylase activity, HMG-CoA reductase activity, (E)-4-hydroxy-3-methylbut-2-enyl-diphosphate reductase activity, 1-deoxy-D-xylose-5-phosphate synthase activity, 1-deoxy-D-xylose-5-phosphate reductoisomerase activity, isopentenyl-diphosphate Δ-isomerase activity, geranyl-diphosphate synthase activity, farnesyl-diphosphate synthase activity, geranylgeranyl-diphosphate synthase activity, phytoene synthase activity, phytoene desaturase activity, zeta-carotene desaturase activity, crtISO activity, FtsZ activity and/or MinD activity can be raised analogously by using the appropriate effect genes.

The nucleic acids described above, encoding a ketolase, β-hydroxylase or ε-cyclase, and the nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate Δ-isomerase, nucleic acids encoding a geranyl-diphosphate synthase, nucleic acids encoding a farnesyl-diphosphate synthase, nucleic acids encoding a geranylgeranyl-diphosphate synthase, nucleic acids encoding a phytoene synthase, nucleic acids encoding a phytoene desaturase, nucleic acids encoding a zeta-carotene desaturase, nucleic acids encoding a crtISO protein, nucleic acids encoding a FtsZ protein and/or nucleic acids encoding a MinD protein are preferably incorporated into expression constructs comprising, under the genetic control of regulatory nucleic acid sequences, a nucleic acid sequence coding for an enzyme of the invention; and vectors comprising at least one of these expression constructs.

Such constructs of the invention preferably comprise a promoter 5′-upstream of the particular coding sequence, and a terminator sequence 3′-downstream, and if appropriate further usual regulatory elements, in particular each operatively linked to the effect gene. “Operative linkage” means the sequential arrangement of promoter, coding sequence (effect gene), terminator and, if appropriate, further regulatory elements in such a way that each of the regulatory elements can fulfil its function in the expression of the coding sequence as intended.

Examples of sequences which can be operatively linked are targeting sequences and translation enhancers, enhancers, polyadenylation signals and the like. Further regulatory elements comprise selectable markers, amplification signals, origins of replication and the like.

In addition to the artificial regulatory sequences, it is possible for the natural regulatory sequences still to be present in front of the actual effect gene. This natural regulation can, if appropriate, be switched off by genetic modification, and the expression of the genes can be raised or lowered. The gene construct may, however, also have a simpler structure, meaning that no additional regulatory signals are inserted in front of the structural gene, and the natural promoter with its regulation is not removed. Instead, the natural regulatory sequence is mutated so that regulation no longer takes place, and gene expression is enhanced or diminished. The nucleic acid sequences may be present in one or more copies in the gene construct.

Examples of useful promoters in microorganisms are: cos, tac, trp, tet, trp-tet, lpp, lac, Ipp-lac, lacIq, T7, T5, T3, gal, trc, ara, SP6, lambda-PR or in the lambda-PL promoter, which are preferably used in Gram-negative bacteria; and the Gram-positive promoters amy and SPO2, or the yeast promoters ADC1, MFa, AC, P-60, CYC1, GAPDH. The use of inducible promoters is particularly preferred, such as, for example, light- and, in particular, temperature-inducible promoters such as the PᵣPₗ promoter.

It is possible in principle to use all natural promoters with their regulatory sequences. It is also possible in addition advantageously to use synthetic promoters.

Said regulatory sequences are intended to make targeted expression of the nucleic acid sequences and protein expression, possible. This may mean, depending on the host organism for example, that the gene is expressed or overexpressed only after induction, or that it is immediately expressed and/or overexpressed.

The regulatory sequences or factors may moreover preferably influence positively, and thus raise or lower, the expression. Thus, enhancement of the regulatory elements can take place advantageously at the level of transcription by using strong transcription signals such as promoters and/or enhancers. However, it is also possible to enhance translation by, for example, improving the stability of the mRNA.

An expression cassette is produced by fusing a suitable promoter to the nucleic acid sequences described above and encoding a ketolase, β-hydroxylase, β-cyclase, HMG-CoA reductase, (E)-4-hydroxy-3-methylbut-2-enyl-diphosphate reductase, 1-deoxy-D-xylose-5-phosphate synthase, 1-deoxy-D-xylose-5-phosphate reductoisomerase, isopentenyl-diphosphate Δ-isomerase, geranyl-diphosphate synthase, farnesyl-diphosphate synthase, geranylgeranyl-diphosphate synthase, phytoene synthase, phytoene desaturase, zeta-carotene desaturase, crtISO protein, FtsZ protein and/or MinD protein and to a terminator or polyadenylation signal. Conventional techniques of recombination and cloning are used for this purpose, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which makes optimal expression of the genes in the host possible. Vectors are well known to the skilled worker and can be found for example in “Cloning Vectors” (Pouwels P. H. et al., Eds, Elsevier, Amsterdam-New York-Oxford, 1985). Vectors mean not only plasmids but also all other vectors known to the skilled worker, such as, for example, phages, viruses such as SV40, CMV, baculovirus and adenovirus, transposons, IS elements, phasmids, cosmids, and linear or circular DNA. These vectors may undergo autonomous replication in the host organism or chromosomal replication.

Examples which may be mentioned of suitable expression vectors are:

Conventional fusion expression vectors such as pGEX (Pharmacia Biotech Inc; Smith, D. B. and Johnson, K. S. (1988) Gene 67:31-40), pMAL (New England Biolabs, Beverly, Mass.) and pRIT 5 (Pharmacia, Piscataway, N.J.), with which respectively glutathione S-transferase (GST), maltose E-binding protein and protein A are fused to the recombinant target protein.

Non-fusion protein expression vectors such as pTrc (Amann et al., (1988) Gene 69:301-315) and pET 11d (Studier et al. Gene Expression Technology: Methods in Enzymology 185, Academic Press, San Diego, Calif. (1990) 60-89) or pBluescript and pUC vectors.

Yeast expression vectors for expression in the yeast S. cerevisiae, such as pYepSec1 (Baldari et al., (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.).

Vectors and methods for constructing vectors suitable for use in other fungi, such as filamentous fungi, comprise those described in detail in: van den Hondel, C. A. M. J. J. & Punt, P. J. (1991)”Gene transfer systems and vector development for filamentous fungi, in: Applied Molecular Genetics of Fungi, J. F. Peberdy et al., editors, pp. 1-28, Cambridge University Press: Cambridge.

Baculovirus vectors available for expression of proteins in cultured insect cells (for example Sf9 cells) comprise the pAc series (Smith et al., (1983) Mol. Cell Biol. 3:2156-2165) and the pVL series (Lucklow and Summers (1989) Virology 170:31-39).

Further suitable expression systems for prokaryotic and eukaryotic cells are described in chapter 16 and 17 of Sambrook, J., Fritsch, E. F. and Maniatis, T., Molecular cloning: A Laboratory Manual, 2nd edition, Cold Spring Harbor Laboratory, Cold Spring Harbor Laboratory Press, Cold Spring Harbor, N.Y., 1989.

The expression constructs or vectors of the invention can be used to produce genetically modified microorganisms which are transformed for example with at least one vector of the invention.

The recombinant constructs of the invention described above are advantageously introduced and expressed in a suitable host system. Cloning and transfection methods familiar to the skilled worker, such as, for example, coprecipitation, protoplast fusion, electroporation, retroviral transfection and the like, are preferably used to bring about expression of said nucleic acids in the particular expression system. Suitable systems are described for example in Current Protocols in Molecular Biology, F. Ausubel et al., editors, Wiley Interscience, New York 1997.

Successfully transformed organisms can be selected through marker genes which are likewise present in the vector or in the expression cassette. Examples of such marker genes are genes for antibiotic resistance and for enzymes which catalyze a color-forming reaction which causes staining of the transformed cell. These can then be selected by automatic cell sorting.

Microorganisms which have been successfully transformed with a vector and which harbor an appropriate antibiotic resistance gene (e.g. G418 or hygromycin) can be selected by appropriate antibiotic-containing media or nutrient media. Marker proteins presented on the cell surface can be used for selection by means of affinity chromatography.

The combination of the host organisms and the vectors appropriate for the organisms, such as plasmids, viruses or phages, such as, for example, plasmids with the RNA polymerase/promoter system, phages 8 or other temperate phages or transposons and/or further advantageous regulatory sequences forms an expression system.

The invention further relates to the genetically modified, non-human organisms, where the activity of a ketolase
E is raised compared with the wild type in the case where the wild-type organism already has a ketolase activity, and
F is caused compared with the wild type in the case where the wild-type organism has no ketolase activity
by the genetic modification, and the ketolase activity which is raised according to E or caused according to F is caused by a ketolase selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.
As stated above, the raising (according to E) or causing (according to F) of the ketolase activity compared with the wild type preferably takes place by raising the gene expression of a nucleic acid encoding a ketolase selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.
In a further preferred embodiment, the raising of the gene expression of a nucleic acid encoding a ketolase takes place by introducing into the organism nucleic acids which encode ketolases selected from the group of
A ketolase comprising the amino acid sequence SEQ. ID. NO. 2 or a sequence derived from this sequence by substitution, insertion or deletion of amino acids and having an identity of at least 80% at the amino acid level with the sequence SEQ. ID. NO. 2,
B ketolase comprising the amino acid sequence SEQ. ID. NO. 10 or a sequence derived from this sequence by substitution, insertion or deletion of amino acids and having an identity of at least 90% at the amino acid level with the sequence SEQ. ID. NO. 10,
C ketolase comprising the amino acid sequence SEQ. ID. NO. 12 or a sequence derived from this sequence by substitution, insertion or deletion of amino acids and having an identity of at least 90% at the amino acid level with the sequence SEQ. ID. NO. 12 or
D ketolase comprising the amino acid sequence SEQ. ID. NO. 14 or a sequence derived from this sequence by substitution, insertion or deletion of amino acids and having an identity of at least 50% at the amino acid level with the sequence SEQ. ID. NO. 14.

Thus, in this embodiment, at least one further ketolase gene of the invention is present, compared with the wild type, in the transgenic organisms of the invention. In this embodiment, the genetically modified organism of the invention preferably has at least one exogenous (=heterologous) nucleic acid of the invention encoding a ketolase, or at least two endogenous nucleic acids of the invention encoding a ketolase:

Preferred embodiments of the organisms and nucleic acids encoding a ketolase are described above in connection with the processes of the invention.

Particularly preferred genetically modified organisms have, as mentioned above, additionally a raised or caused hydroxylase activity and/or β-cyclase activity compared with the wild type. Embodiments which are further preferred are described above in the process of the invention.

Further particularly preferred genetically modified non-human organisms have, as mentioned above, additionally at least one further raised activity compared with the wild type, selected from the group of HMG-CoA reductase activity, (E)-4-hydroxy-3-methylbut-2-enyl-diphosphate reductase activity, 1-deoxy-D-xylose-5-phosphate synthase activity, 1-deoxy-D-xylose-5-phosphate reductoisomerase activity, isopentenyl-diphosphate Δ-isomerase activity, geranyl-diphosphate synthase activity, farnesyl-diphosphate synthase activity, geranylgeranyl-diphosphate synthase activity, phytoene synthase activity, phytoene desaturase activity, zeta-carotene desaturase activity, crtISO activity, FtsZ activity and MinD activity. Further preferred embodiments are described above in the process of the invention.

Further preferred genetically modified plants have, as mentioned above, additionally a reduced ε-cyclase activity compared with a wild-type plant. Further preferred embodiments are described above in the process of the invention.

Organisms mean according to the invention preferably organisms which as wild-type or initial organisms are able, naturally or through genetic complementation and/or rerouting of metabolic pathways, to produce carotenoids, especially β-carotene and/or zeaxanthin and/or neoxanthin and/or violaxanthin and/or lutein.

Further preferred organisms already have as wild-type or initial organisms a hydroxylase activity and are thus able as wild-type or initial organisms to produce zeaxanthin.

Preferred organisms are plants or microorganisms such as, for example, bacteria, yeasts, algae or fungi.

Bacteria which can be used are both bacteria which are able, owing to the introduction of genes of carotenoid biosynthesis from a carotenoid-producing organism, to synthesize xanthophylls, such as, for example, bacteria of the genus Escherichia which comprise, for example, crt genes from Erwinia, and bacteria intrinsically able to synthesize xanthophylls, such as, for example, bacteria of the genus Erwinia, Agrobacterium, Flavobacterium, Alcaligenes, Paracoccus, Nostoc or cyanobacteria of the genus Synechocystis.

Preferred bacteria are Escherichia coli, Erwinia herbicola, Erwinia uredovora, Agrobacterium aurantiacum, Alcaligenes sp. PC-1, Flavobacterium sp. strain R1534, the cyanobacterium Synechocystis sp. PCC6803, Paracoccus marcusii or Paracoccus carotinifaciens.

Preferred yeasts are Candida, Saccharomyces, Hansenula, Pichia or Phaffia. Particularly referred yeasts are Xanthophyllomyces dendrorhous or Phaffia rhodozyma.

Preferred fungi are Aspergillus, Trichoderma, Ashbya, Neurospora, Blakeslea, especially Blakeslea trispora, Phycomyces, Fusarium or further fungi described in Indian Chem. Engr. Section B. Vol. 37, No. 1, 2 (1995) on page 15, Table 6.

Preferred algae are green algae such as, for example, algae of the genus Haematococcus, Phaedactylum tricornatum, Volvox or Dunaliella. Particularly preferred algae are Haematococcus puvialis or Dunaliella bardawil.

Further useful microorganisms and their production for carrying out the process of the invention are disclosed for example in DE-A-199 16 140, which is incorporated herein by reference.

Particularly preferred plants are those selected from the families Amaranthaceae, Amaryllidaceae, Apocynaceae, Asteraceae, Balsaminaceae, Begoniaceae, Berberidaceae, Brassicaceae, Cannabaceae, Caprifoliaceae, Caryophyllaceae, Chenopodiaceae, Compositae, Curcurbitaceae, Cruciferae, Euphorbiaceae, Fabaceae, Gentianaceae, Geraniaceae, Graminae, Illiaceae, Labiatae, Lamiaceae, Leguminosae, Liliaceae, Linaceae, Lobliaceae, Malvaceae, Oleaceae, Orchidaceae, Papaveraceae, Plumbaginaceae, Poaceae, Polemoniaceae, Primulaceae, Ranunculaceae, Rosaceae, Rubiaceae, Scrophulariaceae, Solanaceae, Tropaeolaceae, Umbelliferae, Verbanaceae, Vitaceae and Violaceae.

Very particularly preferred plants are selected from the group of plant genera Marigold, Tagetes errecta, Tagetes patula, Acacia, Aconitum, Adonis, Amica, Aquilegia, Aster, Astragalus, Bignonia, Calendula, Caltha, Campanula, Canna, Centaurea, Chemanthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Labumum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, particularly preferably selected from the group of plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

Very particularly preferred genetically modified plants are selected from the plant genera Marigold, Tagetes erecta, Tagetes patula, Adonis, Lycopersicon, Rosa, Calendula, Physalis, Medicago, Helianthus, Chrysanthemum, Aster, Tulipa, Narcissus, Petunia, Geranium or Tropaeolum, where the genetically modified plant comprises at least one transgene nucleic acid encoding a ketolase.

The transgenic plants, their propagation material, and their plant cells, tissues or parts, especially their fruits, seeds, flowers and petals, are a further aspect of the present invention.

The genetically modified plants can, as described above, be used to produce ketocarotenoids, especially astaxanthin.

Genetically modified organisms, especially plants or plant parts, of the invention which are consumable by humans and animals, such as, in particular, petals with a raised content of ketocarotenoids, especially astaxanthin, can also be used for example directly or after processing known per se as human or animal food or as animal and human dietary supplements.

The genetically modified organisms can also be used to produce ketocarotenoid-containing extracts of the organisms and/or to produce animal and human dietary supplements.

The genetically modified organisms have a raised content of ketocarotenoids by comparison with the wild type.

A raised content of ketocarotenoids ordinarily means a raised total ketocarotenoid content.

However, a raised content of ketocarotenoids also means in particular an altered content of the preferred ketocarotenoids, without the total carotenoid content necessarily being raised.

In a particularly preferred embodiment, the genetically modified plants of the invention have a raised astaxanthin content by comparison with the wild type. A raised content means in this case also a caused content of ketocarotenoids, or astaxanthin.

The invention further relates to a ketolase comprising the amino acid sequence SEQ. ID. NO. 2 or a sequence derived from this sequence by substitution, insertion or deletion of amino acids and having an identity of at least 80%, preferably at least 85%, more preferably at least 90%, more preferably at least 95%, more preferably at least 97%, particularly preferably at least 99%, at the amino acid level with the sequence SEQ. ID. NO. 2.

Preferred ketolases comprise the sequence SEQ. ID. NO. 2, 4, 6 or 8. Particularly preferred ketolases are ketolases having the sequences SEQ. ID. NO. 2, 4, 6 or 8.

The invention further relates to nucleic acids encoding the ketolases described above.

Preferred nucleic acids comprise the sequence SEQ. ID. NO. 1, 3, 5 or 7. Particularly preferred nucleic acids are nucleic acids having the sequence SEQ. ID. NO. 1, 3, 5 or 7.

The invention further relates to a ketolase comprising the amino acid sequence SEQ. ID. NO. 10 or a sequence derived from this sequence by substitution, insertion or deletion of amino acids and having an identity of at least 90%, preferably at least 92%, more preferably at least 95%, more preferably at least 97%, more preferably at least 98%, particularly preferably at least 99%, at the amino acid level with the sequence SEQ. ID. NO. 10.

Preferred ketolases comprise the sequence SEQ. ID. NO. 10. Particularly preferred ketolases are ketolases of the sequence SEQ. ID. NO. 10.

The invention further relates to nucleic acids encoding a ketolase described above.

Preferred nucleic acids comprise the sequence SEQ. ID. NO. 9. Particularly preferred nucleic acids are nucleic acids of the sequence SEQ. ID. NO. 9.

The invention further relates to ketolases comprising the amino acid sequence SEQ. ID. NO. 12 or a sequence derived from this sequence by substitution, insertion or deletion of amino acids and having an identity of at least 90%, preferably at least 92%, more preferably at least 95%, more preferably at least 97%, more preferably at 98%, particularly preferably at least 99%, at the amino acid level with the sequence SEQ. ID. NO. 12.

Preferred ketolases comprise the sequence SEQ. ID. NO. 12. Particularly preferred ketolases are ketolases of the sequence SEQ. ID. NO. 12.

The invention, further relates to nucleic acids encoding a ketolase described above. Preferred nucleic acids comprise the sequence SEQ. ID. NO. 11. Particularly preferred nucleic acids are nucleic acids of the sequence SEQ. ID. NO. 11.

The invention is illustrated by the examples which now follow but is not confined thereto:

General experimental conditions:

### Recombinant DNA sequence analysis

Recombinant DNA molecules were sequenced using a laser fluorescence DNA sequencer from Licor (marketed by MWG Biotech, Ebersbach) by the method of Sanger (Sanger et al., Proc. Natl. Acad. Sci. USA 74 (1977), 5463-5467).

### EXAMPLE 1

### Amplification of a DNA which Encodes the Entire Primary Sequence of the Ketolase NP60.79:BKt from Nostoc punctiforme SAG 60.79

The DNA which codes for the ketolase NP60.79:BKT was amplified by PCR from Nostoc punctiforme SAG 60.79 (SAG: Sammiung von Algenkulturen Göttingen).

To prepare genomic DNA from a suspension culture of Nostoc punctiforme SAG 60.79, which was grown in BG 11 medium (1.5 g/l NaNO3, 0.04 g/l K2PO4×3H2O, 0.075 g/l MgSO4×H2O, 0.036 g/l CaCl2×2H₂O, 0.006 g/l citric acid, 0.006 g/l ferric ammonium citrate, 0.001 g/l EDTA disodium magnesium, 0.04 g/l Na₂CO₃,1 ml trace metal mix A5+Co (2.86 g/l H₃BO3, 1.81 g/l MnCl2×4H2O, 0.222 gA ZnSO4×7H2o, 0.39 g/l NaMoO4×2H2o, 0.079 g/l CuSO4×5H₂O, 0.0494 g/l Co(NO₃)2×6H₂O)) at 25° C. with continuous light and constant shaking (150 rpm) for 1 week, the cells were harvested by centrifugation, frozen in liquid nitrogen and powdered in a mortar.

Protocol for DNA Isolation from Nostoc punctiforme SAG 60.79:

The bacterial cells from a 10 ml liquid culture were pelleted by centrifugation at 8000 rpm for 10 minutes. The bacterial cells were then crushed and ground in liquid nitrogen using a mortar. The cell material was resuspended in 1 ml of 100 mM Tris HCl (pH 7.5) and transferred into an Eppendorf reaction vessel (2 ml volume). After addition of 100 μl of proteinase K (concentration: 20 mg/ml), the cell suspension was incubated at 37° C. for 3 hours. The suspension was then extracted with 50 μl of phenol. After centrifugation at 13 000 rpm for 5 minutes, the upper, aqueous phase was transferred into a new 2 ml Eppendorf reaction vessel. The extraction with phenol was repeated 3 times. The DNA was precipitated by adding 1/10 volume of 3 M sodium acetate (pH 5.2) and 0.6 volume of isopropanol and then washed with 70% ethanol. The DNA pellet was dried at room temperature, taken up in 25 μl of water and dissolved by heating to 65° C.

The nucleic acid coding for the ketolase NP60.79:BKT from Nostoc punctiforme SAG 60.79 was amplified by a polymerase chain reaction (PCR) from Nostoc punctiforme SAG 60.79 using a sense-specific primer (NP196-1, SEQ ID No. 59) and an antisense-specific primer (NP196-2 SEQ ID No. 60).

The PCR conditions were as follows:

The PCR for amplification of the DNA which codes for a ketolase protein consisting of the entire primary sequence took place in a 50 ul reaction mixture which comprised:
1 ul of a Nostoc punctiforme SAG 60.79 DNA (prepared as described above)
0.25 mM dNTPs
0.2 mM NP196-1 (SEQ ID No. 59)
0.2 mM NP196-2 (SEQ ID No. 60)
5 ul of 10×PCR buffer (TAKARA)
0.25 ul of R Taq polymerase (TAKARA)
25.8 ul of distilled water

The PCR was, carried out under the following cycle conditions:.

### 1×94° C. 2 minutes

### 35×94° C. 1 minute

55° C. 1 minute
72° C. 3 minutes

### 1×72° C. 10 minutes

The PCR amplification with SEQ ID No. 59 and SEQ ID No. 60 resulted in a 792 Bp fragment which codes for a protein consisting of the entire primary sequence (SEQ ID No. 61). The amplicon was cloned, using standard methods, into the PCR cloning vector pCR 2.1-TOPO (Invitrogen), and the clone pNP60.79 was obtained.

### EXAMPLE 2

### Preparation of Expression Vectors for Constitutive Expression of the Ketolase

NP60.79:BKT from Nostoc punctiforme SAG 60.79 in Lycopersicon esculentum and Tagetes erecta Expression of the ketolase from Nostoc punctiforme SAG 60.79 in Lycopersicon esculentum and in Tagetes erecta took place under the control of the constitutive promoter FNR (ferredoxin NADPH oxidoreductase) from Arabidopsis thaliana. The expression took place with the pea transit peptide rbcS (Anderson et al-. 1986, Biochem J. 240:709-715).

The DNA fragment which comprises the FNR promoter region −635 to −1 from Arabidopsis thaliana (SEQ ID No. 65) was prepared by means of PCR using genomic DNA (isolated from Arabidopsis thaliana by standard methods) and the primers FNR-1 (SEQ ID No.63) and FNR-2 (SEQ ID No. 64).

The PCR conditions were as follows:

The PCR for amplifying the DNA which comprises the FNR promoter fragment (−635 to −1) took place in a 50 ul reaction mixture which comprised:
100 ng of genomic DNA from A. thaliana
0.25 mM dNTPs
0.2 mM FNR-1 (SEQ ID No. 63)
0.2 mM FNR-2 (SEQ ID No. 64)
5 [symbol] l of 10×PCR buffer (Stratagene)
0.25 [symbol] l of Pfu polymerase (Stratagene)
28.8 [symbol] l of distilled water

The PCR was carried out under the following cycle conditions:

### 1×94° C. 2 minutes

### 35×94° C. 1 minute

50° C. 1 minute
72° C. 1 minute

### 1×72° C. 10 minutes

The 653 bp amplicon (SEQ ID No. 65) was cloned into the PCR-cloning vector pCR 2.1-TOPO (Invitrogen) using standard methods, and the plasmid pFNR was obtained.

Sequencing of the clone pFNR confirmed a sequence which agrees with a sequence segment on chromosome 5 of Arabidopsis thaliana (database entry ABOL 1474) from position 70127 to 69493. The gene starts at base pair 69492 and is annotated as “ferredoxin-NADP+reductase”.

The clone pFNR was therefore used for cloning into the expression vector pJIT117 (Guerineau et al. 1988, Nucl. Acids Res. 16: 11380).

The cloning took place by isolating the 637 bp KpnI-HindIII fragment from pFNR and ligating into the KpnI-HindIII cut vector pJIT117. The clone which the promoter FNR instead of the original promoter d35S is called pJFNR.

The clone pNP60.79 was used for cloning into the expression vector pJFNR (Example 2). The cloning took place by isolating the 790 Bp SphI fragment from pNP60.79 and ligating into the SphI cut vector pJFNR. The clone which comprises the ketolase from Nostoc punctiforme SAG 60.79 in the correct orientation as N-terminal translational fusion with the rbcS transit peptide is called pJFNRNP60.79.

Preparation of an expression cassette for Agrobacterium-mediated transformation of the ketolase NP60.79:BKT from Nostoc punctiforme SAG 60.79 into Lycopersicon esculentum took place using the binary vector pSUN3 (WO02/00900).

To prepare the expression vector pS3FNRNP60.79, the 2.4 Kb KpnI fragment from pJFNRNP60.79 was ligated to the KpnI cut vector pSUN3. This clone is called MSP1.

Preparation of an Expression Cassette for Agrobacterium-Mediated Transformation of the expression vector with the ketolase NP60.79:BKT from Nostoc punctiforme SAG 60.79 in Tagetes erecta took place using the binary vector pSUN5 (WO02/00900).

To prepare the expression vector pS5FNRNP60.69, the 2.4 Kb KpnI fragment from pJFNRNP60.79 was ligated to the KpnI cut vector pSUN5. This clone is called MSP2.

### EXAMPLE 3

### Amplification of a DNA which Encodes the Entire Primary Sequence of the Ketolase

NP60.79:BKT from Nostoc punctiforme SAG 71.79 The DNA which codes for the ketolase NP71.79:BKT was amplified by PCR from Nostoc punctiforme SAG 71.79 (SAG: Sammiung von Algenkulturen Göttingen).

To prepare genomic DNA from a suspension culture of Nostoc punctiforme SAG 71.79, which was grown in BG 11 medium (1.5 g/l NaNO3, 0.04 g/l K2PO4×3H₂O, 0.075 g/l MgSO4×H2O, 0.036 g/l CaCl2×2H2O, 0.006 g/l citric acid, 0.006 g/l ferric ammonium citrate, 0.001 g/l EDTA disodium magnesium, 0.04 g/l Na₂CO3, 1 ml trace metal mix A5+Co (2.86 g/l H₃BO3, 1.81 g/l MnCl2×4H2o, 0.222 g/l ZnSO4×7H2o, 0.39 g/l NaMoO4×2H2o, 0.079 g/l CuSO4×5H2O, 0.0494 g/l Co(NO3)2×6H₂O)) at 25° C. with continuous light and constant shaking (150 rpm) for 1 week, the cells were harvested by centrifugation, frozen in liquid nitrogen and powdered in a mortar.

Protocol for DNA isolation from Nostoc punctiforme SAG 71.79:

The bacterial cells from a 10 ml, liquid culture were pelleted by centrifugation at 8000 rpm for 10 minutes. The bacterial cells were then crushed and ground in liquid nitrogen using a mortar. The cell material was resuspended in 1 ml of 10 mM Tris HCl (pH 7.5) and transferred into an Eppendorf reaction vessel (2 ml volume). After addition of 100 μl of proteinase K (concentration: 20 mg/ml), the cell suspension was incubated at 37° C. for 3 hours. The suspension was then extracted with 500 μl of phenol. After centrifugation at 13 000 rpm for 5 minutes, the upper, aqueous phase was transferred into a new 2 ml Eppendorf reaction vessel. The extraction with phenol was repeated. 3 times. The DNA was precipitated by adding 1/10 volume of 3 M sodium acetate (pH 5.2) and 0.6 volume of isopropanol and then washed with 700% ethanol. The DNA pellet was dried at room temperature, taken up in 25 μl of water and dissolved by heating to 65° C.

The nucleic acid coding for the ketolase NP71.79:BKT from Nostoc punctiforme SAG 71.79 was amplified by a polymerase chain reaction (PCR) from Nostoc punctiforme SAG 71.79 using a sense-specific primer (NP196-1, SEQ ID No. 59) and an antisense-specific primer (NP196-2 SEQ ID No. 60).

The PCR conditions were as follows:

The PCR for amplification of the DNA which codes for a ketolase protein consisting of the entire primary sequence took place in a 50 ul reaction mixture which comprised:
1 ul of a Nostoc punctiforme SAG 71.79 DNA (prepared as described above)
0.25 mM dNTPs
0.2 mM NP196-1 (SEQ ID No. 59)
0.2 mM NP196-2 (SEQ ID No. 60)
5 ul of 10×PCR buffer (TAKARA)
0.25 ul of R Taq polymerase (TAKARA)
25.8 ul of distilled water

The PCR was carried out under the following cycle conditions:

### 1×94° C. 2 minutes

### 35×94° C. 1 minute

55° C. 1 minute
72° C. 3 minutes

### 1×72° C. 10 minutes

The PCR amplification with SEQ ID No. 59 and SEQ ID No. 60 resulted in a 792 Bp fragment which codes for a protein consisting of the entire primary sequence (SEQ ID No. 66). The amplificate was cloned, using standard methods, into the PCR cloning vector pCR 2.1-TOPO (Invitrogen), and the clone pNP71.79 was obtained.

### EXAMPLE 4

### Preparation of Expression Vectors for Constitutive Expression of the Ketolase NP71.79:BKT from Nostoc punctiforme SAG 71.79 in Lycopersicon esculentum and Tagetes erecta

Expression of the ketolase from Nostoc punctiforme SAG 71.79 in Lycopersicon esculentum and in Tagetes erecta took place under the control of the constitutive promoter FNR (ferredoxin NADPH oxidoreductase) from Arabidopsis thaliana. The expression took place with the pea transit peptide rbcS (Anderson et al. 1986, Biochem J. 240:709-715).

The clone pNP71.79 was used for cloning into the expression vector pJFNR (Example 2). The cloning took place by isolating the 790 Bp SphI fragment from pNP71.79 and ligating into the SphI cut vector pJFNR. The clone which comprises the ketolase from Nostoc punctiforme SAG 71.79 in the correct orientation as N-terminal translational fusion with the rbcS transit peptide is called pJFNRNP71.79.

Preparation of an expression cassette for Agrobacterium-mediated transformation of the ketolase NP71.79:BKT from Nostoc punctiforme SAG 71.79 into Lycopersicon esculentum took place using the binary vector pSUN3 (WO02/00900).

To prepare the expression vector pS3FNRNP71.79, the 2.4 Kb KpnI fragment from pJFNRNP71.79 was ligated to the KpnI cut vector pSUN3. This clone is called MSP3.

Preparation of an expression cassette for Agrobacterium-mediated transformation of the expression vector with the ketolase NP71.79:BKT from Nostoc punctiforme SAG 71.79 in Tagetes erecta took place using the binary vector pSUN5 (WO02/00900).

To prepare the expression vector pS5FNRNP71.69, the 2.4 Kb KpnI fragment from pJFNRNP71.79 was ligated to the KpnI cut vector pSUN5. This clone is called MSP4.

### EXAMPLE 5

### Amplification of a DNA which Encodes the Entire Primary Sequence of the Ketolase NS037:BKT from Nodularia spumigena CCAUV 01-037

The DNA which codes for the ketolase NS037:BKT was amplified by PCR from Nodularia spumigena CCAUV 01-037 (CCAUV: Culture Collection of Algae at the University of Vienna).

To prepare genomic DNA from a suspension culture of Nodularia spumigena CCAUV 01-037, which was grown in BG 11 medium (1.5 g/l NaNO3, 0.04 g/l K2PO4×3H₂O, 0.075 g/l MgSO4×H₂O, 0.036 g/l CaCl2×2H₂O, 0.006 g/l citric acid, 0.006 g/l ferric ammonium citrate, 0.001 g/l EDTA disodium magnesium, 0.04 g/l Na2CO3, 1 ml trace metal mix A5+Co (2.86 g/l H₃BO3, 1.81 g/l MnCl2×4H2o, 0.222 g/l ZnSO4×7H2o, 0.39 g/l NaMoO4×2H2o, 0.079 g/l CuSO4×5H₂O, 0.0494 g/l Co(NO₃)2×6H₂O)) at 25° C. with continuous light and constant shaking (150 rpm) for 1 week, the cells were harvested by centrifugation, frozen in liquid nitrogen and powdered in a mortar.

Protocol for DNA isolation from Nodularia spumigena CCAUV 01-037:

The bacterial cells from a 10 ml liquid culture were pelleted by centrifugation at 8000 rpm for 10 minutes. The bacterial cells were then crushed and ground in liquid nitrogen using a mortar. The cell material was resuspended in 1 ml of 10 mM Tris HCl (pH 7.5) and transferred into an Eppendorf reaction vessel (2 ml volume). After addition of 100 μl of proteinase K (concentration: 20 mg/ml), the cell suspension was incubated at 37° C. for 3 hours. The suspension was then extracted with 500 μl of phenol. After centrifugation at 13 000 rpm for 5 minutes, the upper, aqueous phase was transferred into a new 2 ml Eppendorf reaction vessel. The extraction with phenol was repeated 3 times. The DNA was precipitated by adding 1/10 volume of 3 M sodium acetate (pH 5.2) and 0.6 volume of isopropanol and then washed with 70% ethanol. The DNA pellet was dried at room temperature, taken up in 25 μl of water and dissolved by heating to 65° C.

The nucleic acid coding for the ketolase NS037:BKT from Nodularia spumigena CCAUV 01-037 was amplified by a polymerase chain reaction (PCR) from Nodularia spumigena CCAUV 01-037 using a sense-specific primer (NP196-1, SEQ ID No. 59) and an antisense-specific primer (NSK-2 SEQ ID No. 68).

The PCR conditions were as follows:

The PCR for amplification of the DNA which codes for a ketolase protein consisting of the entire primary sequence took place in a 50 ul reaction mixture which comprised:
1 ul of a Nodularia spumigena CCAUV 01-037 DNA (prepared as described above)
0.25 mM dNTPs
0.2 mM NP196-1 (SEQ ID No. 59)
0.2 mM NSK-2 (SEQ ID No. 68)
5 ul of 10×PCR buffer (TAKARA)
0.25 ul of R Taq polymerase (TAKARA)
25.8 ul of distilled water

The PCR was carried out under the following cycle conditions:

### 1×94° C. 2 minutes

### 35×94° C. 1 minute

55° C. 1 minute
72° C. 3 minutes

### 1×72° C. 10 minutes

The PCR amplification with SEQ ID No. 59 and SEQ ID No. 68 resulted in an 807 Bp fragment which codes for a protein consisting of the entire primary sequence (SEQ ID No. 69). The amplicon was cloned, using standard methods, into the PCR cloning vector pCR 2.1-TOPO (Invitrogen), and the clone pNS037 was obtained.

### EXAMPLE 6

### Preparation of Expression Vectors for Constitutive Expression of the Ketolase NS037:BKT from Nodularia spumigena CCAUV 01-037 in Lycopersicon esculentum and Tagetes erecta

Expression of the ketolase from Nodularia spumigena CCAUV 01-037 in Lycopersicon esculentum and in Tagetes erecta took place under the control of the constitutive promoter FNR (ferredoxin NADPH oxidoreductase) from Arabidopsis thaliana. The expression took place with the pea transit peptide rbcS (Anderson et al. 1986, Biochem J. 240:709-715).

The clone pNS037 was used for cloning into the expression vector PJFNR (Example 2). The cloning took place by isolating the 797 Bp SphI fragment from pNS037 and ligating into the SphI cut vector pJFNR. The clone which comprises the ketolase from Nodularia spumigena CCAUV 01-037 in the correct orientation as N-terminal translational fusion with the rbcS transit peptide is called pJFNRNS037.

Preparation of an expression cassette for Agrobacterium-mediated transformation of the ketolase NS037:BKT from Nodularia spumigena CCAUVO1-037 into Lycopersicon esculentum took place using the binary vector pSUN3 (WO02/00900).

To prepare the expression vector pS3FNRNS037, the 2.4 Kb KpnI fragment from pJFNRS037 was ligated to the KpnI cut vector pSUN3. This clone is called MSP5.

Preparation of an expression cassette for Agrobacterium-mediated transformation of the expression vector with the ketolase NS037:BKT from Nodularia spumigena CCAUV 01-037 in Tagetes erecta took place using the binary vector pSUN5 (WO02/00900).

To prepare the expression vector pS5FNRNS037, the 2.4 Kb KpnI fragment from pJFNRNS037 was ligated to the KpnI cut vector pSUN5. This clone is called MSP6.

### EXAMPLE 7

### Amplification of a DNA which Encodes the Entire Primary Sequence of the Ketolase NS053:BKT from Nodularia spumigena CCAUV 01-053

The DNA which codes for the ketolase NS053:BKT was amplified by PCR from Nodularia spumigena CCAUV 01-053 (CCAUV: Culture Collection of Algae at the University of Vienna).

To prepare genomic DNA from a suspension culture of Nodularia spumigena CCAUV 01-053, which was grown in BG 11 medium (1.5 g/l NaNO3, 0.04 g/l K2PO4×3H2O, 0.075 g/l MgSO4×H₂O, 0.036 g/l CaCl₂×2H₂O, 0.006 g/l citric acid, 0.006 g/l ferric ammonium citrate, 0.001 g/l EDTA disodium magnesium, 0.04 g/l Na₂CO3, 1 ml trace metal mix A5+Co (2.86 g/l H₃BO3, 1.81 g/l MnCl2×4H2o, 0.222 g/l ZnSO4×7H2o, 0.39 g/l NaMoO4×2H2o, 0.079 g/l CuSO4×5H₂O, 0.0494 g/l Co(NO₃)2×6H₂O)) at 25° C. with continuous light and constant shaking (150 rpm) for 1 week, the cells were harvested by centrifugation, frozen in liquid nitrogen and powdered in a mortar.

Protocol for DNA isolation from Nodularia spumigena CCAUV 01-053:

The bacterial cells from a 10 ml liquid culture were pelleted by centrifugation at 8000 rpm for 10 minutes. The bacterial cells were then crushed and ground in liquid nitrogen using a mortar. The cell material was resuspended in 1 ml of 10 mM Tris HCl (pH 7.5) and transferred into an Eppendorf reaction vessel (2 ml volume). After addition of 100 μl of proteinase K (concentration: 20 mg/ml), the cell suspension was incubated at 37° C. for 3 hours. The suspension was then extracted with 500 μl of phenol. After centrifugation at 13 000 rpm for 5 minutes, the upper, aqueous phase was transferred into a new 2 ml Eppendorf reaction vessel. The extraction with phenol was repeated 3 times. The DNA was precipitated by adding 1/10 volume of 3 M sodium acetate (pH 5.2) and 0.6 volume of isopropanol and then washed with 70% ethanol. The DNA pellet was dried at room temperature, taken up in 25 μl of water and dissolved by heating to 65° C.

The nucleic acid coding for the ketolase NS053:BKT from Nodularia spumigena CCAUV 01-053 was amplified by a polymerase chain reaction (PCR) from Nodularia spumigena CCAUV 01-053 using a sense-specific primer (NP196-1, SEQ ID No. 59) and an antisense-specific primer (NSK-2 SEQ ID No. 68).

The PCR conditions were as follows:

The PCR for amplification of the DNA which codes for a ketolase protein consisting of the entire primary sequence took place in a 50 ul reaction mixture which comprised:
1 ul of a Nodularia spumigena CCAUV 01-053 DNA (prepared as described above)
0.25 mM dNTPs
0.2 mM NP196-1 (SEQ ID No. 59)
0.2 mM NSK-2 (SEQ ID No. 68)
5 ul of 10×PCR buffer (TAKARA)
0.25 ul of R Taq polymerase (TAKARA)
25.8 ul of distilled water

The PCR was carried out under the following cycle conditions:

### 1×94° C. 2 minutes

### 35×94° C. 1 minute

55° C. 1 minute
72° C. 3 minutes

### 1×72° C. 10 minutes

The PCR amplification with SEQ ID No. 59 and SEQ ID No. 68 resulted in an 807 Bp fragment which codes for a protein consisting of the entire primary sequence (SEQ ID No. 71). The amplicon was cloned, using standard methods, into the PCR cloning vector pCR 2.1-TOPO (Invitrogen), and the clone pNS053 was obtained.

### EXAMPLE 8

### Preparation of Expression Vectors for Constitutive Expression of the Ketolase NS053:BKT from Nodularia spumigena CCAUV 01-053 in Lycopersicon esculentum and Tagetes erecta

Expression of the ketolase from Nodularia spumigena CCAUV 01-053 in Lycopersicon esculentum and in Tagetes erecta took place under the control of the constitutive promoter FNR (ferredoxin NADPH oxidoreductase) from Arabidopsis thaliana. The expression took place with the pea transit peptide rbcS (Anderson et al. 1986, Biochem J. 240:709-715).

The clone pNS053 was used for cloning into the expression vector pJFNR (Example 2). The cloning took place by isolating the 797 Bp SphI fragment from pNS053 and ligating into the SphI cut vector pJFNR. The clone which comprises the ketolase from Nodularia spumigena CCAUV 01-053 in the correct orientation as N-terminal translational fusion with the rbcS transit peptide is called pJFNRNS053.

Preparation of an expression cassette for Agrobacterium-mediated transformation of the ketolase NS053:BKT from Nodularia spumigena CCAUV 01-053 into Lycopersicon esculentum took place using the binary vector pSUN3 (WO02/00900).

To prepare the expression vector pS3FNRNS053, the 2.4 Kb KpnI fragment from pJFNRS053 was ligated to the KpnI cut vector pSUN3. This clone is called MSP7.

Preparation of an expression cassette for Agrobacterium-mediated transformation of the expression vector with the ketolase NS053:BKT from Nodularia spumigena CCAUV 01-053 in Tagetes erecta took place using the binary vector pSUN5 (WO02/00900).

To prepare the expression vector pS5FNRNS053, the 2.4 Kb KpnI fragment from pJFNRNS053 was ligated to the KpnI cut vector pSUN5. This clone is called MSP8.

### EXAMPLE 9

### Amplification of a DNA which Encodes the Entire Primary Sequence of the Ketolase GV35.87:BKT from Gloeobacter violaceus SAG 35.87

The DNA which codes for the ketolase GV35.87:BKT was amplified by PCR from Gloeobacter violaceus SAG 35.87 (SAG: Sammlung von Algenkulturen Göttingen).

To prepare genomic DNA from a suspension culture of Gloeobacter violaceus SAG 35.87, which was grown in BG 11 medium (1.5 g/l NaNO3, 0.04 g/l K2PO4×3H₂O, 0.075 g/l MgSO4×H₂O, 0.036 g/l CaCl₂×2H₂O, 0.006 g/l citric acid, 0.006 g/l ferric ammonium citrate, 0.001 g/l EDTA disodium magnesium, 0.04 g/l Na2CO3, 1 ml trace metal mix A5+Co (2.86 g/l H₃BO3, 1.81 g/l MnCl2×4H2o, 0.222 g/l ZnSO4×7H2o, 0.39 g/l NaMoO4×2H2o, 0.079 g/l CuSO4×5H₂O, 0.0494 g/l Co(NO3)2×6H₂O)) at 25° C. with continuous light and constant shaking (150 rpm) for 1 week, the cells were harvested by centrifugation, frozen in liquid nitrogen and powdered in a mortar.

Protocol for DNA isolation from Gloeobacter violaceus SAG 35.87:

The bacterial cells from a 10 ml liquid culture were pelleted by centrifugation at 8000 rpm for 10 minutes. The bacterial cells were then crushed and ground in liquid nitrogen using a mortar. The cell material was resuspended in 1 ml of 10 mM Tris HCl (pH 7.5) and transferred into an Eppendorf reaction vessel (2 ml volume). After addition of 100 μl of proteinase K (concentration: 20 mg/ml), the cell suspension was incubated at 37° C. for 3 hours. The suspension was then extracted with 500 μl of phenol. After centrifugation at 13 000 rpm for 5 minutes, the upper, aqueous phase was transferred into a new 2 ml Eppendorf reaction vessel. The extraction with phenol was repeated 3 times. The DNA was precipitated by adding 1/10 volume of 3 M sodium acetate (pH 5.2) and 0.6 volume of isopropanol and then washed with 70% ethanol. The DNA pellet was dried at room temperature, taken up in 25 μl of water and dissolved by heating to 65° C.

The nucleic acid coding for the ketolase GV35.87:BKT from Gloeobacter violaceus SAG 35.87 was amplified by a polymerase chain reaction (PCR) from Gloeobacter violaceus SAG 35.87 using a sense-specific primer (GVK-F1, SEQ ID No. 73) and an antisense-specific primer (GVK-R1SEQ ID No. 74).

The PCR conditions were as follows:

The PCR for amplification of the DNA which codes for a ketolase protein consisting of the entire primary sequence took place in a 50 ul reaction mixture which comprised:
1 ul of a Gloeobacter violaceus SAG 35.87 DNA (prepared as described above)
0.25 mM dNTPs
0.2 mM GVK-F1 (SEQ ID No. 73)
0.2 mM GVK-R1 (SEQ ID No. 74)
5 ul of 10×PCR buffer (TAKARA)
0.25 ul of R Taq polymerase (TAKARA)
25.8 ul of distilled water

The PCR was carried out under the following cycle conditions:

### 1×94° C. 2 minutes

### 35×94° C. 1 minute

55° C. 1 minute
72° C. 3 minutes

### 1×72° C. 10 minutes

The PCR amplification with SEQ ID No. 73 and SEQ ID No. 74 resulted in a 785 Bp fragment which codes for a protein consisting of the entire primary sequence (SEQ ID No. 75). The amplicon was cloned, using standard methods, into the PCR cloning vector pCR 2.1-TOPO (invitrogen), and the clone pGV35.87 was obtained.

### EXAMPLE 10

### Preparation of Expression Vectors for Constitutive Expression of the Ketolase GV35.87:BKT from Gloeobacter violaceus SAG 35.87 in Lycopersicon esculentum and Tagetes erecta

Expression of the ketolase from Gloeobacter violaceus SAG 35.87 in Lycopersicon esculentum and in Tagetes erecta took place under the control of the constitutive promoter FNR (ferredoxin NADPH oxidoreductase) from Arabidopsis thaliana. The expression took place with the pea transit peptide rbcS (Anderson et al. 1986, Biochem J. 240:709-715).

The clone pGV35.87 was used for cloning into the expression vector pJFNR (Example 2). The cloning took place by isolating the 797 Bp SphI fragment from pGV35.87 and ligating into the SphI cut vector pJFNR. The clone which comprises the ketolase from Gloeobacter violaceus SAG 35.87 in the correct orientation as N-terminal translational fusion with the rbcS transit peptide is called pJFNRGV35.87.

Preparation of an expression cassette for Agrobacterium-mediated transformation of the ketolase GV35.87:BKT from Gloeobacter violaceus SAG 35.87 into Lycopersicon esculentum took place using the binary vector pSUN3 (WO02/00900).

To prepare the expression vector pS3FNRGV35.87, the 2.4 Kb KpnI fragment (partial KpnI hydrolysis) from pJFNRGV35.87 was ligated to the KpnI cut vector pSUN3. This clone is called MSP9.

Preparation of an expression cassette for Agrobacterium-mediated transformation of the expression vector with the ketolase GV35.87:BKT from Gloeobacter violaceus SAG 35.87 in Tagetes erecta took place using the binary vector pSUN5 (WO02/00900).

To prepare the expression vector pS5FNRGV35.87, the 22.4 Kb KpnI fragment (partial KpnI hydrolysis) from pJFNRGV35.87 was ligated to the KpnI cut vector pSUN5. This clone is called MSP10.

### EXAMPLE 11

### Construction of the Plasmid pMCL-CrtYIBZ/idi/gps for Synthesizing Zeaxanthin in E. coli

Construction of pMCL-CrtYIBZ/idi/gps took place in three steps via the intermediate stages of pMCL-CrtYIBZ and pMCL-CrtYIBZ/idi. The vector used was the plasmid pMCL200 which is compatible with high copy number vectors (Nakano, Y., Yoshida, Y., Yamashita, Y. and Koga, T.; Construction of a series of pACYC-derived plasmid vectors; Gene 162 (1995), 157-158).

### EXAMPLE 11.1

### Construction of pMCL-CrtYIBZ

The biosynthesis genes crtY, crtB, crtI and crtZ are derived from the bacterium Erwinia uredovora and were amplified by PCR. Genomic DNA from Erwinia uredovora (DSM 30080) was provided by the preparation service of the Deutsche Sammlung von Microorganismen und Zellkuturen (DSMZ, Brunswick). The PCR reaction was carried out in accordance with the manufacturer's information (Roche, Long Template PCR: Procedure for amplification of 5-20 kb targets with the expand long template PCR system). The PCR conditions for amplifying the Erwinia uredovora biosynthesis cluster were as follows:

Master Mix 1:
1.75 [symbol] l dNTPs (final concentration 350 μM)
0.3 μM primer Crt1 (SEQ ID No. 77)
0.3 μM primer Crt2 (SEQ ID No. 78)
250-500 ng of genomic DNA from DSM 30080 distilled water to a total volume of 50 μl

Master Mix 2:
5 ul of 10× PCR buffer 1 (final concentration 1×, with 1.75 mM Mg2+)
10× PCR buffer 2 (final concentration 1×, with 2.25 mM Mg2+)
10× PCR buffer 3 (final concentration 1×, with 2.25 mM Mg2+)
0.75 ul of Expand Long Template Enzyme Mix (final concentration 2.6 Units) distilled water to a total volume of 50 μl

The two mixtures “Master Mix 1” and “Master Mix 2” were pipetted together. The PCR was carried out in a total volume of 50 ul under the following cycle conditions:

### 1×94° C. 2 minutes

### 30×94° C. 30 seconds

58° C. 1 minute
68° C. 4 minutes

### 1×72° C. 10 minutes

The PCR amplification with SEQ ID No. 77 and SEQ ID No. 78 resulted in a fragment (SEQ ID NO. 79) which codes for the genes CrtY (protein: SEQ ID NO. 80), CrtI (protein: SEQ ID NO. 81), crtB (protein: SEQ ID NO. 82) and CrtZ (iDNA). The amplicon was cloned into the PCR cloning vector pCR2.1 (Invitrogen) by using standard methods, and the clone pCR2.1-CrtYIBZ was obtained.

The plasmid pCR2.1-CrtYIBZ was SalI and HindIII cut, and the resulting SalI/HindIII fragment was isolated and transferred by ligation into the SalI/HindIII cut vector pMCL200. The SalI/HindIII fragment from pCR2.1-CrtYIBZ cloned into pMCL 200 is 4624 Bp long, codes for the CrtY, CrtI, crtB and CrtZ genes and corresponds to the sequence from position 2295 to 6918 in D90087 (SEQ ID No. 79). The CrtZ gene is transcribed by means of its endogenous promoter contrary to the direction of reading of the CrtY, CrtI and CrtB genes. The resulting clone is called pMCL-CrtYIBZ.

### EXAMPLE 11.2

### Construction of pMCL-CrtYIBZ/idi

The gene idi (isopentenyl-diphosphate isomerase; IPP isomerase) was amplified from E. coli by PCR. The nucleic acid encoding the entire idi gene with idi promoter and ribosome binding site was amplified from E. coli by a polymerase chain reaction (PCR) using a sense-specific primer (5′-idi SEQ ID No. 81) and an antisense-specific primer (3′-idi SEQ ID No. 82).

The PCR conditions were as follows:

The PCR for amplifying the DNA took place in a 50 μl reaction mixture which comprised:
1 [symbol] l of an E. coli TOP10 suspension
0.25 mM dNTPs
0.2 mM 5′-idi (SEQ ID No. 81)
0.2 mM 3′-idi (SEQ ID No. 82)
5 [symbol] l of 10×PCR buffer (TAKARA)
0.25 [symbol] l of R Taq polymerase (TAKARA)
28.8 [symbol] l of distilled water

The PCR was carried out under the following cycle conditions:

### 1×94° C. 2 minutes

### 20×94° C. 1 minute

62° C. 1 minute
72° C. 1 minute

### 1×72° C. 10 minutes

The PCR amplification with SEQ ID No. 81 and SEQ ID No. 82 resulted in a 679 Bp fragment which codes for a protein consisting of the entire primary sequence (SEQ ID No. 83). The amplicon was cloned into the PCR cloning vector pCR2.1 (Invitrogen) using standard methods, and the clone pCR2.1-idi was obtained.

Sequencing of the clone pCR2.1-idi confirmed a sequence which does not differ from the published sequence AE000372 in position 8774 to position 9440. This region comprises the promoter region, the potential ribosome binding site and the entire open reading frame for the IPP isomerase. The fragment cloned into pCR2.1-idi has, owing to the insertion of an XhoI cleavage site at the 5′ end and of a SalI cleavage site at the 3′ end of the idi gene, a total length of 679 Bp.

This clone was therefore used to clone the idi gene into the vector pMCL-CrtYIBZ. The cloning took place by isolating the XhoI/SalI fragmente from pCR2.1-idi. and ligating into the XhoI/SalI cut vector pMCL-CrtYIBZ. The resulting clone is called pMCL-CrtYIBZ/idi.

### EXAMPLE 11.3

### Construction of pMCL-CrtYIBZ/idi/gps

The gene gps (geranylgeranyl-pyrophosphate synthase; GGPP synthase) was amplified from Archaeoglobus fulgidus by PCR. The nucleic acid encoding gps from Archaeoglobus fulgidus was amplified by a polymerase chain reaction (PCR) using a sense-specific primer (5′-gps SEQ ID No. 85) and an antisense-specific primer (3′-gps SEQ ID No. 86).

The DNA from Archaeoglobus fulgidus was provided by the preparation service of the Deutsche Sammiung von Microorganismen und Zellkulturen (DSMZ, Brunswick). The PCR conditions were as follows:

The PCR for amplifying the DNA which codes for a GGPP synthase protein consisting of the entire primary sequence took place in a 50 μl reaction mixture which comprised:
1 [symbol] l of an Archaeoglobus fulgidus DNA
0.25 mM dNTPs
0.2 mM 5′-gps (SEQ ID No. 85)
0.2 mM 3′-gps (SEQ ID No. 86)
5 [symbol] l of 10×PCR buffer (TAKARA)
0.25 [symbol] l of R Taq polymerase (TAKARA)
28.8 [symbol] l of distilled water

The PCR was carried out under the following cycle conditions:

### 1×94° C. 2 minutes

### 20×94° C. 1 minute

56° C. 1 minute
72° C. 1 minute

### 1×72° C. 10 minutes

The DNA fragment amplified by PCR and the primers SEQ ID No. 85 and SEQ ID No. 86 was eluted by methods known per se from the agarose gel and cut with the restriction enzymes NcoI and HindIII. This resulted in a 962 Bp fragment which codes for a protein consisting of the entire primary sequence (SEQ ID No. 87). The NcoI/HindIII cut amplicon was cloned by using standard methods into the vector pCB97-30, and the clone pCB-gps was obtained.

Sequencing the clone pCB-gps confirmed a sequence for the GGPP synthase from A. fulgidus which differs from the published sequence AF120272 in one nucleotide. The second codon of the GGPP synthase was altered by the insertion of an NcoI cleavage site in the gps gene. In the published sequence AF120272, CTG (position 4-6) codes for leucine. The amplification with the two primers SEQ ID No. 85 and SEQ ID No. 86 changed this second codon to GTG, which codes for valine.

The clone pCB-gps was therefore used for cloning the gps gene into the vector pMCL-CrtYIBZ/idi. The cloning took place by isolating the KpnI/XhoI fragment from pCB-gps and ligating into the KpnI and XhoI cut vector pMCL-CrtYIBZ/idi. The cloned KpnI/XhoI fragment harbors the Prrn16 promoter together with a minimal 5′-UTR sequence of rbcL, the first 6 codons of rbcL which extend the GGPP synthase N-terminally, and 3′ of the gps gene the psbA sequence. The N terminus of the GGPP synthase thus has instead of the natural amino acid sequence with Met-Leu-Lys-Glu (amino acid 1 to 4 from AF120272) the altered amino acid sequence Met-Thr-Pro-Gln-Thr-Ala-Met-Val-Lys-Glu. The result of this is that the recombinant GGPP synthase is identical starting with Lys in position 3 (in AF120272) and shows no further alterations in the amino acid sequence. The rbcL and psbA sequences were used in accordance with a reference by Eibl et al. (Plant J. 19. (1999), 1-13). The resulting clone is called pMCL-CrtYI BZ/idi/gps.

### EXAMPLE 12

Biotransformation of Zeaxanthin in Recombinant E. coli Strains

For the zeaxanthin biotransformation, recombinant E. coli strains which are able, through heterologous complementation, to produce zeaxanthin were prepared. Strains of E. coli TOP10 were used as host cells for the complementation experiments with the plasmids i) pNP60.79:BKT or ii) pNP71.79:BKT or iii) pNS037:BKT and pMCL-CrtYIBZ/idi/gps as second plasmid.

In order to prepare E. coli strains which make it possible to synthesize zeaxanthin in high concentration, the plasmid pMCL-CrtYIBZ/idi/gps was constructed. The plasmid harbors the biosynthesis genes crtY, crtB, crtI and crtY from Erwinia uredovora, the gene gps (for geranylgeranyl-pyrophoshate synthastase) from Archaeoglobus fulgidus and the gene idi (isopentenyl-diphosphate isomerase) from E. coli. This construct was used to eliminate limiting steps for high accumulation of carotenoids and their biosynthetic precursors. This has been described previously by Wang et al. in a similar manner with a plurality of plasmids (Wang, C.-W., Oh, M.-K. and Liao, J. C.; Engineered isoprenoid pathway enhances astaxanthin production in Escherichia coli, Biotechnology and Bioengineering 62 (1999), 235-241).

Cultures of E. coli TOP10 were transformed in a manner known per se with the plasmids pMCL-CrtYIBZ/idi/gps and i) pNP60.79:BKT, or ii) pNP71.79:BKT or iii) pNS037:BKT and cultivated in LB medium at 30° C. or 37° C. overnight. Ampicillin (50 μg/ml), chloramphenicol (50 μg/ml) and isopropyl α-thiogalactoside (1 mmol) were added in a manner which is usual per se, likewise overnight The E. coli cultures thus harbored in each case a low copy number and a high copy number plasmid.

Carotenoids were isolated from the recombinant strains by extracting the cells with acetone, evaporating the organic solvent to dryness and fractionating the carotenoids by HPLC on a C30 column. The following process conditions were set.

Separating column: Prontosil C30 column, 250×4,6 mm, (Bischoff, Leonberg)
Flow rate: 1.0 ml/min

### Eluents: mobile phase A—100% methanol

mobile phase B—80% methanol, 0.2% ammonium acetate
mobile phase C—100% t-butyl methyl ether
   Gradient profile:

| | | % mobile | % mobile | % mobile |
|---|---|---|---|---|
| Time | Flow rate | phase A | phase B | phase C |
| 1.00 | 1.0 | 95.0 | 5.0 | 0 |
| 1.05 | 1.0 | 80.0 | 5.0 | 15.0 |
| 14.00 | 1.0 | 42.0 | 5.0 | 53.0 |
| 14.05 | 1.0 | 95.0 | 5.0 | 0 |
| 17.00 | 1.0 | 95.0 | 5.0 | 0 |
| 18.00 | 1.0 | 95.0 | 5.0 | 0 |

### Detection: 300-500 nm

The spectra were determined directly from the eluted peaks using a photodiode array detector. The isolated substances were identified from their absorption spectra and their retention times by comparison with standard samples.

The ketolase from Nostoc punctiforme 71.79 was expressed using the plasmid pNP71.79:BKT, the ketolase from Nostoc punctiforme 60.79 was expressed using the plasmid pNP60.79:BKT and the ketolase from Nodularia spumigena was expressed using pNS037:BKT. Determinations at the wavelength 600 nm before extraction of the carotenoids showed a total cell count of 6.1×10⁹ for E. coli/pNP71.79:BKT, a total cell count of 6.3×10⁹ for E. coli/pNP60.79:BKT and a total cell count of 6.2×10⁹ for E. coli/pNS037:BKT.

Table 1 shows a comparison of the amounts of bacterially produced carotenoids:

TABLE 1
Concentration of carotenoids extracted from E. coli in ng/ml of culture.

| E: coli | | | | | | | | | |
|---|---|---|---|---|---|---|---|---|---|
| expressing | | | | | | | | | |
| ketolase | | | | | | | | | Total |
| from | Cantha | Adoniru | Adonixa | Asta | Zea | Crypto | Beta-C | Total | Ketocaro |
| NP 71.79 | 208 | 11 | 13 | 52 | 305 | 161 | 1388 | 2140 | 284 |
| NP 60.79 | 1490 | 337 | 10 | 89 | 0 | 60 | 1322 | 3308 | 1926 |
| NS 037 | 45 | 0 | 148 | 1038 | 457 | 218 | 285 | 2190 | 1230 |

Abbreviations:
cantha for canthaxanthin,
adonir for adonirubin,
adonix for adonixanthin,
asta for astaxanthin,
zea for zeaxanthin,
crypto for beta-cryptoxanthin,
beta-C for beta-carotene,
total for totral carotenoid content,
total ketocaro: total of all the ketocarotenoids

The total carotenoid concentration after incubation for about 18 hours was about ⅓ higher in E. coli/pNP60.79:BKT than in E. coli/pNP71.79:BKT. The amount of ketocarotenoids (canthaxanthin, adonirubin, adonixanthin and astaxanthin) was 1966 ng in E. coli/pNP60.79:BKT, which was distinctly higher than the 284 ng in E. coli/pNP71.79:BKT, the cell count being the same. The proportion of ketocarotenoids is 58% in E. coli/pNP60.79:BKT and only 13% in E. coli/pNP71.79:BKT, in each case based on the total carotenoid content. The proportion of the ketocarotenoids is 56% in E. coli/pNsO37:BKT based on the total carotenoid content.

### EXAMPLE 13

### Production of Transgenic Lycopersicon esculentum Plants

Tomato plants were transformed and regenerated by the published method of Ling and coworkers (Plant Cell Reports (1998), 17:843-847). A higher kanamycin concentration was used to select the Microtom variety (100 mg/L).

Cotyledons and hypocotyls of seven- to ten-day old seedlings of the Microtom line were used as initial explant for the transformation. The culture medium of Murashige and Skoog (1962: Murashige and Skoog, 19,62, Physiol. Plant 15, 473-) with 2% sucrose, pH 6.1, was used for germination. The germination took place at 21° C. with a low light level (20-100 μE). After seven to ten days, the cotyledons were divided transversely, and the hypocotyls were cut into segments about 5-10 mm long and placed on the MSBN medium (MS, pH 6.1, 3% sucrose+1 mg/l BAP, 0.1 mg/l NM) which were charged the preceding day with suspension-cultured tomato cells. The tomato cells were covered, free of air bubbles, with sterile filter paper. The preculture of the explants on the described medium took place for three to five days. Cells of the Agrobacterium tumefaciens LBA4404 strain were transformed singly with the plasmids pS3FNRNP60.79, pS3FNRNP71.79, pS3FNRNS037, pS3FNRNS053, pS3FNRGV35.87. Each of the individual agrobacterium strains transformed with the binary vectors pS3FNRNP60.79, pS3FNRNP71.79, pS3FNRNS037, pS3FNRNS053, pS3FNRGV35.87 was cultivated in an overnight culture in YEB medium with kanamycin (20 mg/l) at 28° C., and the cells were centrifuged. The bacterial pellet was resuspended with liquid MS medium (3% sucrose, pH 6,1) and adjusted to an optical density of 0.3 (at 600 nm). The precultured explants were transferred into the suspension and incubated at room temperature with gentle shaking for 30 minutes. The explants were then dried with sterile filter paper and returned to their preculture medium for the three-day coculture (21° C.).

After the coculture, the explants were transferred to MSZ2 medium (MS pH 6.1+3% sucrose, 2 mg/l zeatin, 100 mg/l kanamycin, 160 mg/l Timentin) and stored under low light conditions (20-100 gE, 16 h/8 h light rhythm) at 21° C. for the selective regeneration. The explants were transferred every two to three weeks until shoots formed. Small shoots could be detached from the explant and rooted on MS (pH 6.1+3% sucrose) 160 mg/l Timentin, 30 mg/l kanamycin, 0.1 mg/l IAA. Rooted plants were transferred into a glasshouse.

The following lines were obtained by the transformation method described above using the following expression constructs:

pS3FNRNP60.79 resulted in: MSP1-1, MSP1-2, MSP1-3
pS3FNRNP71.79 resulted in: MSP3-1, MSP3-2, MSP3-3
pS3FNRNS037 resulted in: MSP5-1, MSP5-2, MSP5-3
pS3FNRNS053 resulted in: MSP7-1, MSP7-2, MSP7-3
pS3FNRGV35.87 resulted in: MSP9-1, MSP9-2, MSP9-3

### EXAMPLE 14

Production of Transgenic Tagetes Plants

Tagetes seeds are sterilized and placed on germination medium (MS medium; Murashige and Skoog, Physiol. Plant. 15 (1962), 473-497) pH 5.8, 2% sucrose). The germination takes place in a temperature/light/time interval of 18-28° C./20-200 μE/3-16 weeks, but preferably at 21° C., 20-70 μE, for 4-8 weeks.

All the leaves of the in vitro plants which have developed by then are harvested and cut transverse to the midrib. The leaf explants produced thereby with a size of 10-60 mm² are stored during the preparation in liquid MS medium at room temperature for a maximum of 2 h.

Any Agrobacterium tumefaciens strain, but preferably a supervirulent strain, such as, for example, EHA105 with an appropriate binary plasmid which may harbor a selection marker gene (preferably bar or pat) and one or more trait genes or reporter genes is (pS5FNRNP60.79, pS5FNRNP71.79, pS5FNRNS037, pS5FNRNS053, pS5FNRGV35.87) cultured overnight and used for coculturing with the leaf material. The culturing of the bacterial strain can take place as follows: a single colony of the appropriate strain is inoculated in YEB (0.1% yeast extract, 0.5% beef extract, 0.5% peptone, 0.5% sucrose, 0.5% magnesium sulfate×7H₂O) with 25 mg/l kanamycin and cultured at 28° C. for 16 to 20 h. The bacterial suspension is then harvested by centrifugation at 6000 g for 10 min and resuspended in liquid MS medium in such a way that an OD₆₀₀ of about 0.1 to 0.8 result. This suspension is used for the coculturing with the leaf material.

Immediately before the cocultivation, the MS medium in which the leaves have been stored is replaced by the suspension of bacteria. Incubation of the leaflets in the suspension of agrobacteria took place at room temperature with gentle shaking for 30 min. The infected explants are then placed on MS medium which has been solidified with agar e.g. 0.8% plant agar (Duchefa, N L) and comprises growth regulators such as, for example, 3 mg/l benzylaminopurine (BAP) and 1 mg/l indolylacetic acid (IAA). The orientation of the leaves on the medium has no significance. The explants are cultivated for 1 to 8 days, but preferably for 6 days, using the following conditions in this case: light intensity: 30-80 μMol/m²×sec, temperature: 22-24° C., 16/8 hours light/dark alternation. The cocultivated explants are then transferred to fresh MS medium, preferably comprising the same growth regulators, this second medium additionally comprising an antibiotic to suppress bacterial growth. Timentin in a concentration of from 200 to 500 mg/l is very suitable for this purpose. The second selective component employed is one to select for successful transformation. Phosphinothricin in a concentration of from 1 to 5 mg/l selects very efficiently, but other selective components are conceivable according to the process used.

After from one to three weeks in each case, the explants are transferred to fresh medium until shoot buds and small shoots develop, which are then transferred to the same basal medium including timentin and PPT or alternative components with growth regulators, namely, for example, 0.5 mg/l indolylbutyric acid (IBA) and 0.5 mg/l gibberillic acid GA₃, for rooting. Rooted shoots can be transferred to a glasshouse.

In addition to the method described, the following advantageous modifications are possible:

Before the explants are infected with the bacteria, they can be preincubated for from 1 to 12 days, preferably 3-4, on the medium described above for the coculture. This is followed by infection, coculture and selective regeneration as described above.

The pH for the regeneration (normally 5.8) can be reduced to pH 5.2. This improves control of the growth of agrobacteria.

Addition of AgNO₃ (3-10 mg/l) to the regeneration medium improves the condition of the culture, including the regeneration itself.

Components which reduce phenol formation and are known to the skilled worker, such as, for example, citric acid, ascorbic acid, PVP and many others, have a positive effect on the culture.

Liquid culture medium can also be used for the overall process. The culture can also be incubated on commercially available supports which are positioned on the liquid medium.

The following lines were obtained by the transformation method described above using the following expression constructs:

pS5FNRNP60.79 resulted in: MSP2-1, MSP2-2, MSP2-3
pS5FNRNP71.79 resulted in: MSP4-1, MSP4-2, MSP4-3
pS5FNRNS037 resulted in: MSP6-1, MSP6-2, MSP6-3
pS5FNRNS053 resulted in: MSP8-1, MSP8-2, MSP8-3
pS5FNRGV35.87 resulted in: MSP10-1, MSP10-2, MSP10-3

### EXAMPLE 15

### Enzymatic Lipase-Catalyzed Hydrolysis of the Carotenoid Esters from Plant Material and Identification of the Carotenoids

### General Procedure

a) Ground plant material (e.g. petal material) (30-100 mg fresh weight) is extracted with 100% acetone (three times 500 μl; shake for about 15 minutes each time). The solvent is evaporated. Carotenoids are then taken up in 495 μl of acetone, 4.95 ml of potassium phosphate buffer (100 mM, pH7.4) are added and thoroughly mixed. This is followed by addition of about 17 mg of bile salts (Sigma) and 149 μl of an NaCl/CaCl₂ solution (3M NaCl and 75 mM CaCl₂). The suspension is incubated at 37° C. for 30 minutes. For the enzymatic hydrolysis of the carotenoid esters, 595 μl of a lipase solution (50 mg/ml lipase type 7 from Candida rugosa (Sigma)) are added and incubated at 37 C with shaking. After about 21 hours, 595 μl of lipase are again added, and incubation is continued at 37° C. for at least 5 hours. Approximately about 700 mg of Na₂SO₄ are then dissolved in the solution. After addition of 1800 μl of petroleum ether, the carotenoids are extracted into the organic phase by vigorous mixing. This extraction is repeated until the organic phase remains colorless. The petroleum ether fractions are combined, and the petroleum ether is evaporated. Free carotenoids are taken up in 100-120 μl of acetone. Free carotenoids can be identified by means of HPLC and C30 reverse phase column on the basis of the retention time and UV-VIS spectra.

The bile salts or bile acid salts used are 1:1 mixtures of cholate and deoxycholate.

b) Procedure for workup if only small amounts of carotenoid esters are present in the plant material

Alternatively, the carotenoid esters can be hydrolyzed by Candida rugosa lipase after separation by means of thin-layer chromatography. For this purpose, 50-100 mg of plant material are extracted three times with about 750 μl of acetone. The solvent extract is concentrated in rotary evaporator in vacuo (raised temperatures of 40-50° C. are tolerable). This is followed by addition of 300 μl of petroleum ether:acetone (ratio 5:1) and thorough mixing. Suspended materials are sedimented by centrifugation (1-2 minutes). The upper phase is transferred into a new reaction vessel. The remaining residue is again extracted with 200 μl of petroleum ether:acetone (ratio 5:1), and suspended materials are removed by centrifugation. The two extracts are put together (volume 500 μl) and the solvents are evaporated. The residue is resuspended in 30 μl of petroleum ether:acetone (ratio 5:1) and loaded onto a thin-layer plate (silica gel 60, Merck). If more than loading is necessary for preparative-analytical purposes, several aliquots each with a fresh weight of 50-100 mg should be prepared in the described manner for the separation by thin-layer chromatography. The thin-layer plate is developed in petroleum ether:acetone (ratio 5:1). Carotenoid bands can be identified visually on the basis of their color. Individual carotenoid bands are scraped off and can be pooled for preparative-analytical purposes. The carotenoids are eluted from the silica material with acetone; the solvent is evaporated in vacuo. To hydrolyze the carotenoid esters, the residue is dissolved in 495 μl of acetone, and 17 mg of bile salts (Sigma), 4.95 ml of 0.1M potassium phosphate buffer (pH 7.4) and 149 μl (3M NaCl, 75 mM CaCl₂) are added. After thorough mixing, the mixture is equilibrated at 37° C. for 30 min. This is followed by addition of 595 μl of Candida rugosa lipase (Sigma, stock solution of 50 mg/ml in 5 mM CaCl₂). Incubation with lipase takes place overnight with shaking at 37° C. After about 21 hours, the same amount of lipase is again added; incubation is continued with shaking at 37° C. for at least 5 hours. This is followed by addition of 700 mg of Na₂SO₄ (anhydrous); extraction is carried out with 1800 μl petroleum ether for about 1 minute, and the mixture is centrifuged at 3500 revolutions/minute for 5 minutes. The upper phase is transferred into a new reaction vessel, and the extraction is continued until the upper phase is colorless. The combined petroleum ether phase is concentrated in vacuo (temperatures of 40-50° C. are possible). The residue is dissolved in 120 μl of acetone, possibly using ultrasound. The dissolved carotenoids can be separated by HPLC using a C30 column and quantified by means of reference substances.

### EXAMPLE 16

### HPLC Analysis of Free Carotenoids

The samples obtained by the procedures in Example 15 are analyzed under the following conditions:

The following HPLC conditions were set.

Separating column: Prontosil C30 column, 250×4,6 mm, (Bischoff, Leonberg, Germany)
Flow rate: 1.0 ml/min

### Eluents: mobile phase A—100% methanol

mobile phase B—80% methanol, 0.2% ammonium acetate
mobile phase C—100% t-butyl methyl ether

### Detection: 300-530 nm

Gradient profile:

| | | % mobile | % mobile | % mobile |
|---|---|---|---|---|
| Time | Flow rate | phase A | phase B | phase C |
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids produced according to the invention are, for example:

violaxanthin 11.7 min, astaxanthin 17.7 min, adonixanthin 19 min, adonirubin 19.9 min, zeaxanthin 21 min.

1-87. (canceled)

88. A process for preparing ketocarotenoids by cultivating a genetically modified, non-human organism which, by comparison with the wild type, have a modified ketolase activity, and the modified ketolase activity is caused by a ketolase selected from the group consisting of:
A. ketolase comprising the amino acid sequence SEQ ID NO: 2 or a sequence derived from this sequence by substitution, insertion or deletion of amino acids and having an identity of at least 80% at the amino acid level with the sequence SEQ ID NO: 2,
B. ketolase comprising the amino acid sequence SEQ ID NO: 10 or a sequence derived from this sequence by substitution, insertion or deletion of amino acids and having an identity of at least 90% at the amino acid level with the sequence SEQ ID NO: 10,
C. ketolase comprising the amino acid sequence SEQ ID NO: 12 or a sequence derived from this sequence by substitution, insertion or deletion of amino acids and having an identity of at least 90% at the amino acid level with the sequence SEQ ID NO: 12, and
D. ketolase comprising the amino acid sequence SEQ ID NO: 14 or a sequence derived from this sequence by substitution, insertion or deletion of amino acids and having an identity of at least 50% at the amino acid level with the sequence SEQ ID NO: 14.

89. The process according to claim 88, wherein the non-human organism which already has a ketolase activity as wild type is used, and the genetic modification brings about a raising of the ketolase activity by comparison with the wild type.

90. The process according to claim 89, wherein the ketolase activity is raised by raising the gene expression of a nucleic acid encoding a ketolase selected from the group consisting of:
A. ketolase comprising the amino acid sequence SEQ ID NO: 2 or a sequence derived from this sequence by substitution, insertion or deletion of amino acids and having an identity of at least 80% at the amino acid level with the sequence SEQ ID NO: 2,
B. ketolase comprising the amino acid sequence SEQ ID NO: 10 or a sequence derived from this sequence by substitution, insertion or deletion of amino acids and having an identity of at least 90% at the amino acid level with the sequence SEQ ID NO: 10,
C. ketolase comprising the amino acid sequence SEQ ID NO: 12 or a sequence derived from this sequence by substitution, insertion or deletion of amino acids and having an identity of at least 90% at the amino acid level with the sequence SEQ ID NO: 12, and
D. ketolase comprising the amino acid sequence SEQ ID NO: 14 or a sequence derived from this sequence by substitution, insertion or deletion of amino acids and having an identity of at least 50% at the amino acid level with the sequence SEQ ID NO: 14,
compared with the wild type.

91. The process according to claim 90, wherein the gene expression is raised by introducing into the organism a nucleic acid which encodes a ketolase selected from the group consisting of:
A. ketolase comprising the amino acid sequence SEQ ID NO: 2 or a sequence derived from this sequence by substitution, insertion or deletion of amino acids and having an identity of at least 80% at the amino acid level with the sequence SEQ ID NO: 2,
B. ketolase comprising the amino acid sequence SEQ ID NO: 10 or a sequence derived from this sequence by substitution, insertion or deletion of amino acids and having an identity of at least 90% at the amino acid level with the sequence SEQ ID NO: 10,
C. ketolase comprising the amino acid sequence SEQ ID NO: 12 or a sequence derived from this sequence by substitution, insertion or deletion of amino acids and having an identity of at least 90% at the amino acid level with the sequence SEQ ID NO: 12, and
D. ketolase comprising the amino acid sequence SEQ ID NO: 14 or a sequence derived from this sequence by substitution, insertion or deletion of amino acids and having an identity of at least 50% at the amino acid level with the sequence SEQ ID NO: 14.

92. The process according to claim 88, wherein the non-human organism which has no ketolase activity as the wild type is used, and the genetic modification causes a ketolase activity by comparison with the wild type.

93. The process according to claim 92, wherein the genetically modified organism which transgenically expresses a ketolase selected from the group consisting of:
A. ketolase comprising the amino acid sequence SEQ ID NO: 2 or a sequence derived from this sequence by substitution, insertion or deletion of amino acids and having an identity of at least 80% at the amino acid level with the sequence SEQ ID NO: 2,
B. ketolase comprising the amino acid sequence SEQ ID NO: 10 or a sequence derived from this sequence by substitution, insertion or deletion of amino acids and having an identity of at least 90% at the amino acid level with the sequence SEQ ID NO: 10,
C. ketolase comprising the amino acid sequence SEQ ID NO: 12 or a sequence derived from this sequence by substitution, insertion or deletion of amino acids and having an identity of at least 90% at the amino acid level with the sequence SEQ ID NO: 12, and
D. ketolase comprising the amino acid sequence SEQ ID NO: 14 or a sequence derived from this sequence by substitution, insertion or deletion of amino acids and having an identity of at least 50% at the amino acid level with the sequence SEQ ID NO: 14,
is used.

94. The process according to claim 93, wherein the gene expression is caused by introducing into the organism nucleic acids which encode ketolases selected from the group consisting of:
A. ketolase comprising the amino acid sequence SEQ ID NO: 2 or a sequence derived from this sequence by substitution, insertion or deletion of amino acids and having an identity of at least 80% at the amino acid level with the sequence SEQ ID NO: 2,
B. ketolase comprising the amino acid sequence SEQ ID NO: 10 or a sequence derived from this sequence by substitution, insertion or deletion of amino acids and having an identity of at least 90% at the amino acid level with the sequence SEQ ID NO: 10,
C. ketolase comprising the amino acid sequence SEQ ID NO: 12 or a sequence derived from this sequence by substitution, insertion or deletion of amino acids and having an identity of at least 90% at the amino acid level with the sequence SEQ ID NO: 12, and
D. ketolase comprising the amino acid sequence SEQ ID) NO: 14 or a sequence derived from this sequence by substitution, insertion or deletion of amino acids and having an identity of at least 50% at the amino acid level with the sequence SEQ ID NO: 14.

95. The process according to claim 92, feature A, wherein a nucleic acid comprising the sequence of SEQ ID NO: 1, 3, 5 or 7 is introduced.

96. The process according to claim 94, feature A, wherein a nucleic acid comprising the sequence of SEQ ID NO: 1, 3, 5 or 7 is introduced.

97. The process according to claim 92, feature B, wherein a nucleic acid comprising the sequence of SEQ ID NO: 9 is introduced.

98. The process according to claim 94, feature B, wherein a nucleic acid comprising the sequence of SEQ ID NO: 9 is introduced.

99. A genetically modified, non-human organism, wherein the activity of a ketolase
(1) is raised as compared with the wild type in the case where the wild-type organism already has a ketolase activity, or
(2) is caused compared with the wild type in the case where the wild-type organism has no ketolase activity
by the genetic modification, and the ketolase activity which is raised according to (1) or caused according to (2) is caused by a ketolase selected from the group consisting of.
A. ketolase comprising the amino acid sequence SEQ ID NO: 2 or a sequence derived from this sequence by substitution, insertion or deletion of amino acids and having an identity of at least 80% at the amino acid level with the sequence SEQ ID NO: 2,
B. ketolase comprising the amino acid sequence SEQ ID NO: 10 or a sequence derived from this sequence by substitution, insertion or deletion of amino acids and having an identity of at least 90% at the amino acid level with the sequence SEQ ID NO: 10,
C. ketolase comprising the amino acid sequence SEQ ID NO: 12 or a sequence derived from this sequence by substitution, insertion or deletion of amino acids and having an identity of at least 90% at the amino acid level with the sequence SEQ ID NO: 12, and
D. ketolase comprising the amino acid sequence SEQ ID NO: 14 or a sequence derived from this sequence by substitution, insertion or deletion of amino acids and having an identity of at least 50% at the amino acid level with the sequence SEQ ID NO: 14.
